(19) United States
(12) Patent Application Publication
(10) Pub. No.: US 2010/0261710 A1
(43) Pub. Date: Oct. 14, 2010

(54) HDAC Inhibitors

(75) Inventors: Mark A. Ashwell, Carlisle, MA (US); Manish Tandon, Framingham, MA (US); Nivedita D. Namdev, Westford, MA (US); Jean-Marc Lapierre, Pelham, NH (US); Yanbin Liu, Acton, MA (US); Hui Wu, Malden, MA (US)

Correspondence Address: Sunstein Kann Murphy & Timbers LLP, 125 SUMMER STREET, BOSTON, MA, 02110-1618, US

(73) Assignee: ARQULE, INC., Woburn, MA (US)

(21) Appl. No.: 12/671,351

(22) PCT Filed: Aug. 21, 2008
(86) PCT No.: PCT/US08/73873

§ 371 Date: May 20, 2010

Related U.S. Application Data
(60) Provisional application No. 60/965,584, filed on Aug. 21, 2007.

(51) Int. Cl. A61K 31/5517; C07D 487/12; C07D 455/06; A61K 31/437; A61K 31/425; C07D 513/04; A61P 35/04

(52) U.S. Cl. 514/220; 540/561; 546/95; 514/294; 514/368; 548/154

(57) ABSTRACT

The present invention provides hydroxamic acid compounds, and methods of preparation of these compounds. The present invention also relates to pharmaceutical compositions comprising the hydroxamic acid compounds. The present invention provides methods of treating a cell proliferative disorder, such as a cancer, by administering to a subject in need thereof a therapeutically effective amount of a compound of the present invention.

### CROSS-REFERENCE TO RELATED APPLICATIONS

This application claims the benefit of U.S. Provisional Applications No. 60/965,584, filed Aug. 21, 2007, the contents of which are incorporated herein by reference in their entirety.

### BACKGROUND OF THE INVENTION

Cancer is the second leading cause of death in the United States, exceeded only by heart disease. (Cancer Facts and Figures 2004, American Cancer Society, Inc.) Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

Cancer cells are by definition heterogeneous. For example, within a single tissue or cell type, multiple mutational ‘mechanisms’ may lead to the development of cancer. As such, heterogeneity frequently exists between cancer cells taken from tumors of the same tissue and same type that have originated in different individuals. Frequently observed mutational ‘mechanisms’ associated with some cancers may differ between one tissue type and another (e.g., frequently observed mutational ‘mechanisms’ leading to colon cancer may differ from frequently observed ‘mechanisms’ leading to leukemias). It is therefore often difficult to predict whether a particular cancer will respond to a particular chemotherapeutic agent (Cancer Medicine, 5th Edition, Bast et al. eds., B.C. Decker Inc., Hamilton, Ontario).

Breast cancer, for example, is the most frequently diagnosed non-skin cancer in women, and ranks second among cancer deaths in women, after lung cancer (Cancer Facts and Figures 2004, American Cancer Society, Inc.). Current treatment options for breast cancer include surgery, radiotherapy, and chemotherapy/hormone therapy with agents such as tamoxifen, aromatase inhibitors, HERCEPTIN® (trastuzumab), TAXOL® (paclitaxel), cyclophosphamide, methotrexate, doxorubicin (adriamycin), and 5-fluoruracil. Despite improvements in cancer diagnostics and therapeutics, breast cancer incidence rates have continued to increase since 1980. In 2004, about 215,000 new cases of breast cancer are expected in women, and about 1,450 new cases of breast cancer are expected in men. Accordingly, new compounds and methods for treating breast cancer are needed.

Improving the specificity of agents used to treat cancer is of considerable interest because of the therapeutic benefits which would be realized if the side effects associated with the administration of these agents could be reduced. One approach for cancer treatment is targeting histone deactylases (HDACs).

Transcriptional regulation is influenced by the way the DNA is packaged within the cell. The nucleosome, the fundamental block unit, consisting of DNA and histones, is subjected to posttranslational modifications such as methylation, phosphorylation and acetylation. Hyperacetylation, increased levels of histone acetylation, leads to an increase in gene expression, while hypoacetylation, decreased levels of acetylation, suppresses gene expression. The levels of histone acetylation are regulated by two families of enzymes histone acetyltransferases (HATs) and histone deactylases (HDACs). (Cell Cycle, 2004, 3(6), 779)

Currently 18 members of the HDAC superfamily have been identified, spanning three structurally and functionally diverse classes. (Diabetes Metab. Res. Rev., 2005, 21, 416) These enzymes are involved in many aspects of cell and tissue life, many of which are involved in oncology and cell cycling. In addition to histones, HDACs can also deacetylate proteins, such as HSP90, p53, E2F and others involved in various aspects of cell growth. (Cell Cycle, 2004, 3(6), 779) Inhibition of HDACs, inducting hyperacetylation of histones and transcriptional regulation, has been shown to induce growth arrest, differentiation and apoptosis in cancer cells both in vitro and in vivo. (Cell Cycle, 2004, 3(6), 779).

Several HDAC inhibitors are already in the clinical trials as anticancer agents, such as trichostatin A (TSA) and suberoylanilide hydroxamic acid (SAHA), which have been shown to induce differentiation and/or apoptosis in vitro and inhibit tumor growth in mouse models. (Cell Cycle, 2004, 3(6), 779).

There is a need for the development of more HDAC inhibitors for the treatment of cancer.

The references cited herein are not admitted to be prior art to the claimed invention.

### SUMMARY OF THE INVENTION

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### DETAILED DESCRIPTION OF THE INVENTION

### 1. The Hydroxamic Acid Compounds

The present invention provides a compound of formula I:

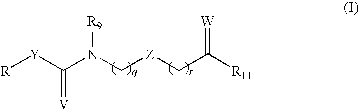

wherein

R is

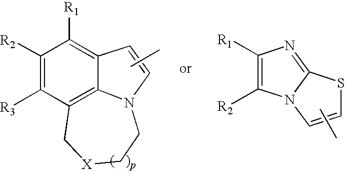

R₁, R₂, and R₃ are each independently selected from the group consisting of H, C₁-C₅ alkyl, C₁-C₅ substituted alkyl, aryl, halogen, —C(═O)NHR₄, and —C(═O)OR₄;

R₄ is H or C₁-C₅ alkyl, aryl, heteroaryl;

p and q are each independently selected from the group consisting of 0, 1, 2, and 3;

X is a bond, NR₅, or S or O;

R₅ is selected from the group consisting of H, alkyl, substituted alkyl, aryl, —CH₂-aryl, heteroaryl, —C(═O)R₆, —C(═O)OR₆, —C(═O)NR₆R₇, —S(═O)2R₆, —(CH₂)ₛOH, and —CH₂CHOHR₆;

R₆ is selected from the group consisting of alkyl, aryl, —CH₂-aryl, heteroaryl;

R₇ is H or C₁-C₅ alkyl; R₆ and R₇ can form a five to seven membered saturated ring;

s is selected from the group consisting of 0, 1, 2, 3, 4, and 5;

Y is a bond, C(═O), or NR₈;

R₈ is H or C₁-C₅ alkyl;

V and W are each independently O or S;

R₉ is selected from the group consisting of H, C₁-C₃ alkyl, aryl, and —CH₂-aryl; or R₉ can form a five or six membered saturated ring with R₁₀;

r is selected from the group consisting of 0, 1, 2, 3, 4, and 5;

Z is selected from the group consisting of a bond, —CHR₁₀, aryl, and alkylene;

R₁₀ is H or C₁-C₅ alkyl;

R₁₁ is —NR₁₂R₁₃, or C₁-C₄ alkyl; and

R₁₂ and R₁₃ are each independently selected from the group consisting of H, hydroxyl, substituted aryl, and heteroaryl.

In an embodiment, R is

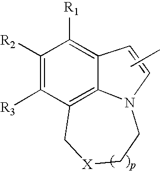

In a further embodiment, R₁, R₂, and R₃ are all H.

In a further embodiment, X is a bond and p is 1. In another embodiment, X is NR₂

In an alternative embodiment, R is

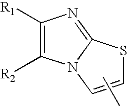

In a further embodiment, R₂ is H.

In an embodiment, both V and W are O.

In an embodiment, R₉ is H. In another embodiment, R₉ is —CH₂-aryl. In another embodiment, R₉ forms a six membered saturated ring with R₁₀.

In an embodiment, Z is aryl. In another embodiment, Z is phenyl. In another embodiment, Z is a bond, q is 1, and r is 1, 2, 3, 4, or 5.

In an embodiment, R₁₁ is —NR₁₂R₁₃. In a further embodiment, R₁₂ is H. In an even further embodiment, R₁₃ is hydroxyl. In an alternative embodiment, R₁₃ is substituted aryl.

In an embodiment, R₁₁ is C₁-C₄ alkyl.

In an embodiment, R₁₁ is methyl

Some representative compounds of Formula I are shown as follows:

The compound can be selected from the group consisting of N-[6-(hydroxyamino)-6-oxohexyl]-5,6-dihydro-4H-pyrrolo[3,2,1-ij]quinoline-1-carboxamide; N-[7-(hydroxyamino)-7-oxoheptyl]-5,6-dihydro-4H-pyrrolo[3,2,1-ij]quinoline-1-carboxamide; N-[8-(hydroxyamino)-8-oxooctyl]-5,6-dihydro-4H-pyrrolo[3,2,1-ij]quinoline-1-carboxamide; N-[5-(hydroxyamino)-5-oxopentyl]-5,6-dihydro-4H-pyrrolo[3,2,1-ij]quinoline-1-carboxamide; N-{4-[(hydroxyamino)carbonyl]benzyl}-5,6-dihydro-4H-pyrrolo[3,2,1-ij]quinoline-1-carboxamide; 6-{[5,6-dihydro-4H-pyrrolo[3,2,1-ij]quinolin-1-yl(oxo)acetyl]amino}-N-hydroxypropanamide, 6-{[5,6-dihydro-4H-pyrrolo[3,2,1-ij]quinolin-1-yl(oxo)acetyl]amino}-N-hydroxyhexanamide; 4-{[5,6-dihydro-4H-pyrrolo[3,2,1-ij]quinolin-1-yl(oxo)acetyl]amino}-N-hydroxybutanamide; 4-{[5,6-dihydro-4H-pyrrolo[3,2,1-ij]quinolin-1-yl(oxo)acetyl]amino}-N-hydroxypentanamide; 7-[(5,6-dihydro-4H-pyrrolo[3,2,1-ij]quinolin-1-ylcarbamoyl)amino]-N-hydroxyheptanamide; 7-{[5,6-dihydro-4H-pyrrolo[3,2,1-ij]quinolin-1-yl(oxo)acetyl]amino}-N-hydroxyheptanamide; 6-[(5,6-dihydro-4H-pyrrolo[3,2,1-ij]quinolin-1-ylcarbamoyl)amino]-N-hydroxyhexanamide; N-benzyl-N-[7-(hydroxyamino)-7-oxoheptyl]-5,6-dihydro-4H-pyrrolo[3,2,1-ij]quinoline-1-carboxamide; tert-butyl 7-{[4-(hydroxycarbamoyl)benzyl]carbamoyl}-3,4-dihydro[1,4]diazepino[6,7,1-hi]indole-2(1H)-carboxylate; N-{4-[2-(hydroxyamino)-2-oxoethyl]phenyl}-5,6-dihydro-4H-pyrrolo[3,2,1-ij]quinoline-1-carboxamide; N-(6-oxoheptyl)-5,6-dihydro-4H-pyrrolo[3,2,1-ij]quinoline-1-carboxamide; 3-[1-(5,6-dihydro-4H-pyrrolo[3,2,1-ij]quinolin-1-ylcarbonyl)piperidin-4-yl]-N-hydroxypropanamide; 4-[1-(5,6-dihydro-4H-pyrrolo[3,2,1-ij]quinolin-1-ylcarbonyl)piperidin-4-yl]butan-2-one; N-{7-[(2-aminophenyl)amino]-7-oxoheptyl}-5,6-dihydro-4H-pyrrolo[3,2,1-ij]quinoline-1-carboxamide; N-{7-[(2-amino-4,5-dichlorophenyl)amino]-7-oxoheptyl}-5,6-dihydro-4H-pyrrolo[3,2,1-ij]quinoline-1-carboxamide; N-[7-(hydroxyamino)-7-oxoheptyl]-6-(3-methoxyphenyl)imidazo[2,1-b][1,3]thiazole-2-carboxamide; and N-[7-(hydroxyamino)-7-oxoheptyl]-5,6-dihydro-4H-pyrrolo[3,2,1-ij]quinoline-2-carboxamide.

Representative compounds of the present invention are also shown in the Examples.

Unless defined otherwise, all technical and scientific terms used herein have the same meaning as commonly understood by one of ordinary skill in the art to which this invention belongs. In case of a conflict in terminology, the present specification controls. The following terms generally have the following meanings.

As used herein, the term “alkyl” includes saturated aliphatic groups, including straight-chain alkyl groups (e.g., methyl, ethyl, propyl, butyl, pentyl, hexyl, heptyl, octyl, nonyl, decyl), branched-chain alkyl groups (e.g., isopropyl, tert-butyl, isobutyl). “Alkyl” further includes alkyl groups that have oxygen, nitrogen, or sulfur atoms replacing one or more hydrocarbon backbone carbon atoms. In certain embodiments, a straight chain or branched alkyl has six or fewer carbon atoms in its backbone (e.g., C₁-C₆ for straight chain, C₃-C₆ for branched chain), and more preferably four or fewer.

The term “alkyl” also includes both “unsubstituted” and “substituted alkyls”, the latter of which refers to alkyl moieties having substituents replacing a hydrogen on one or more carbon of the hydrocarbon backbone. Such substitutents can include, for example, alkyl, alkenyl, alkynyl, hydroxyl, alkylcarbonyl, arylcarbonyl, alkoxycarbonyl, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl, and ureido), amidino, imino, sulfhydryl, alkylthio, arylthio, thiocarboxylate, sulfates, alkylsulfinyl, sulfonato, sulfamoyl (S(O)₂NH₂), aminesulfoxide (NHS(O) or S(O)NH), sulfonamide (NHS(O)₂ or S(O)₂NH), nitro, —CF₃, halogen, cyano, azido, heterocyclyl, alkylaryl, or an aromatic or heteroaromatic moiety. An “alkylaryl” or aralkyl moiety is an alkyl moiety substituted with an aryl (e.g., methylphenyl (benzyl)). “Alkyl” also includes the side chains of natural and unnatural amino acids.

Aryl includes groups with aromaticity, including 5- and 6-membered “unconjugated”, or single-ring aromatic groups that may include from one to four heteroatoms, as well as “conjugated”, or multicyclic systems with at least one aromatic ring. Examples of aryl groups include phenyl, pyrrole, furan, thiophene, thiazole, isothiazole, imidazole, triazole, tetrazole, pyrazole, oxazole, isoxazole, pyridine, pyrazine, pyridazine, and pyrimidine, and the like. Furthermore, the term “aryl” includes multicyclic groups, e.g., tricyclic, bicyclic, e.g., naphthalene, benzoxazole, benzodioxazole, benzothizole, benzoimidazole, benzothiophene, methylenedioxyphenyl, quinoline, isoquinoline, napthridine, indole, benzofuran, purine, benzofuran, deazapureine, or indolizine. Those aryl groups having heteroatoms in the ring structure may also be referred to as “aryl heterocycles”, “heterocycles”, “heterocyclyls”, “heteroaryls” or “heteroaromatics” e.g., pyridine, pyrazole, pyrimidine, furan, isoxazole, imidazole[2,1,b]thiazole, triazole, pyrazine, benzothiophene, imidazole, or thiophene.

The aromatic ring can be substituted at one or more ring positions with such substituents as described above, as for example, halogen, hydroxyl, alkyl, alkoxy, alkylcarbonyloxy, arylcarbonyloxy, alkoxycarbonyloxy, aryloxycarbonyloxy, carboxylate, carboxyacid, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, carboxyalkyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups, which include oxygen, nitrogen, or sulfur replacing one or more hydrocarbon backbone carbons. In certain embodiments, a straight chain or branched chain alkenyl group has six or fewer carbon atoms in its backbone (e.g., C₂-C₆ for straight chain, C₃-C₆ for branched chain.) Likewise, cycloalkenyl groups may have from three to eight carbon atoms in their ring structure, and more preferably have five or six carbons in the ring structure. The term “C₂-C₆” includes alkenyl groups containing two to six carbon atoms.

The term “alkenyl” also includes both “unsubstituted alkenyls” and “substituted alkenyls”, the latter of which refers to alkenyl moieties having substituents replacing a hydrogen on one or more hydrocarbon backbone carbon atoms. Such substituents can include, for example, alkyl groups, alkenyl groups, alkynyl groups, halogens, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, phenyl, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), branched chain alkynyl groups, and cycloalkyl or cycloalkenyl substituted alkynyl groups. The term “alkynyl” further includes alkynyl groups having oxygen, nitrogen, sulfur or phosphorous atoms replacing one or more hydrocarbon backbone carbons. In certain embodiments, a straight chain or branched chain alkynyl group has six or fewer carbon atoms in its backbone (e.g., C₂-C₆ for straight chain, C₃-C₆ for branched chain). The term “C₂-C₆” includes alkynyl groups containing two to six carbon atoms.

The term “alkynyl” also includes both “unsubstituted alkynyls” and “substituted alkynyls”, the latter of which refers to alkynyl moieties having substituents replacing a hydrogen on one or more hydrocarbon backbone carbon atoms. Such substituents can include, for example, alkyl groups, alkenyl groups, alkynyl groups, halogens, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” includes an alkyl group, as defined above, but having from one to ten, more preferably from one to six, carbon atoms in its backbone structure. “Lower alkenyl” and “lower alkynyl” have chain lengths of, for example, 2-5 carbon atoms.

As used herein, “amine” or “amino” includes compounds where a nitrogen atom is covalently bonded to at least one carbon or heteroatom. “Alkylamino” includes groups of compounds wherein nitrogen is bound to at least one additional alkyl group. Examples of alkylamino groups include benzylamino, methylamino, ethylamino, and phenethylamino. “Dialkylamino” includes groups wherein the nitrogen atom is bound to at least two additional alkyl groups. Examples of dialkylamino groups include dimethylamino and diethylamino. “Arylamino” and “diarylamino” include groups wherein the nitrogen is bound to at least one or two aryl groups, respectively. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl, or alkynyl groups bound to an amino group bound to a carboxy group. It includes arylaminocarboxy groups that include aryl or heteroaryl moieties bound to an amino group that is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl, or heterocycle. Substituents on amide groups may be further substituted.

“Acyl” includes compounds and moieties that contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Acylamino” includes moieties wherein an acyl moiety is bonded to an amino group. For example, the term includes alkylcarbonylamino, arylcarbonylamino, carbamoyl and ureido groups.

The term “alkoxy” or “alkoxyl” includes substituted and unsubstituted alkyl, alkenyl, and alkynyl groups covalently linked to an oxygen atom. Examples of alkoxy groups (or alkoxyl radicals) include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, and trichloromethoxy.

The term “cycloalkyl” includes saturated acyclic groups (e.g., cyclopropyl, cyclopentyl, cyclohexyl, cyclohexyl, cycloheptyl, cyclooctyl). Preferred cycloalkyls have from three to eight carbon atoms in their ring structure, and more preferably have five or six carbon atoms in the ring structure. Cycloalkyls includes both “unsubstituted cycloalkyls” and “substituted cycloalkyls”, the latter of which refers to replacing a hydrogen on one or more of the carbons in the ring structure. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The terms “heterocyclyl” or “heterocyclic group” include closed ring structures, e.g., 3- to 10-, or 4- to 7-membered rings, which include one or more heteroatoms. “Heteroatom” includes atoms of any element other than carbon or hydrogen. Examples of heteroatoms include nitrogen, oxygen, or sulfur.

Heterocyclyl groups can be saturated or unsaturated and include pyrrolidine, pyrazine, pyrimidine, oxolane, 1,3-dioxolane, thiolane, tetrahydrofuran, tetrahydropyran, piperidine, piperazine, pyrrolidine, morpholine, lactones, lactams such as azetidinones and pyrrolidinones, sultams, and sultones. Heterocyclic groups such as pyrrole and furan can have aromatic character. They include fused ring structures such as quinoline and isoquinoline. Other examples of heterocyclic groups include pyridine and purine. The heterocyclic ring can be substituted at one or more positions with such substituents as described above, as for example, halogen, hydroxyl, alkylcarbonyloxy, arylcarbonyloxy, alkoxycarbonyloxy, aryloxycarbonyloxy, carboxylate, carboxyacid, alkylcarbonyl, alkoxycarbonyl, aminocarbonyl, alkylthiocarbonyl, alkoxyl, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. Heterocyclic groups can also be substituted at one or more constituent atoms with, for example, a lower alkyl, a lower alkenyl, a lower alkoxy, a lower alkylthio, a lower alkylamino, a lower alkylcarboxyl, a nitro, a hydroxyl, —CF₃, or —CN, or the like.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “carbonyl” or “carboxy” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “thiocarbonyl” or “thiocarboxy” includes compounds and moieties which contain a carbon connected with a double bond to a sulfur atom.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The term “C1-C6” includes one to six carbon atoms (C1, C2, C3, C4, C5 or C6). The term “C2-C6” includes two to six carbon atoms (C2, C3, C4, C5 or C6). The term “C3-C6” includes three to six carbon atoms (C3, C4, C5 or C6). The term “C3-C8” includes two to eight carbon atoms (C3, C4, C5, C6, C7 or

C8). The term “C5-C8” includes five to eight carbon atoms (C5, C6, C7 or C8).

It should be noted that any heteroatom or carbon atom with unsatisfied valences is assumed to have the hydrogen atom to satisfy the valences.

The compounds described herein may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic, and geometric isomeric forms of a structure are intended, unless the specific stereochemistry or isomeric form is specifically indicated. All tautomers of shown or described compounds are also considered to be part of the present invention.

It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

The term “substituted,” as used herein, means that any one or more hydrogens on the designated atom is replaced with a selection from the indicated group, provided that the designated atom's normal valency is not exceeded, and that the substitution results in a stable compound. When a substituent is keto(i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such substituent. Combinations of substituents and/or variables are permissible, but only if such combinations result in stable compounds.

In the specification, the singular forms also include the plural, unless the context clearly dictates otherwise.

### 2. The Synthesis of Hydroxamic Acid Compounds

The present invention also provides methods for the synthesis of the compounds of Formula I. In one embodiment, the present invention provides a method for the synthesis of compounds according to the following schemes, and the protocols shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester, or prodrug thereof.

Compounds of the invention can be prepared in a variety of ways, some of which are known in the art. In general, the compounds of the present invention can be prepared from commercially available starting materials, compounds known in the literature, or from readily-prepared intermediates, by employing standard synthetic methods and procedures known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}.; John Wiley & Sons: New York, 1999, incorporated by reference herein, are useful and recognized reference textbooks of organic synthesis known to those in the art. The following descriptions of synthetic methods are designed to illustrate, but not limit, general procedures for the preparation of compounds of the invention.

The compounds of this invention with general formula I may be prepared according to the following schemes from commercially available starting materials or starting materials, which can be prepared using literature procedures. These schemes show the preparation of representative compounds of this invention.

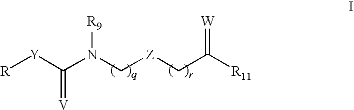

Where R can be,

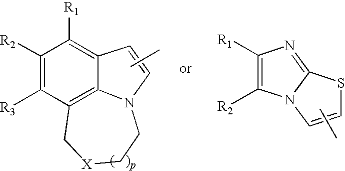

The compounds of the formula I in present invention where W is O and R11 is NHOH or NHAr can be prepared from the reaction of carboxylic acid II where R is IIa, IIb, IIc and IId (Scheme 1).

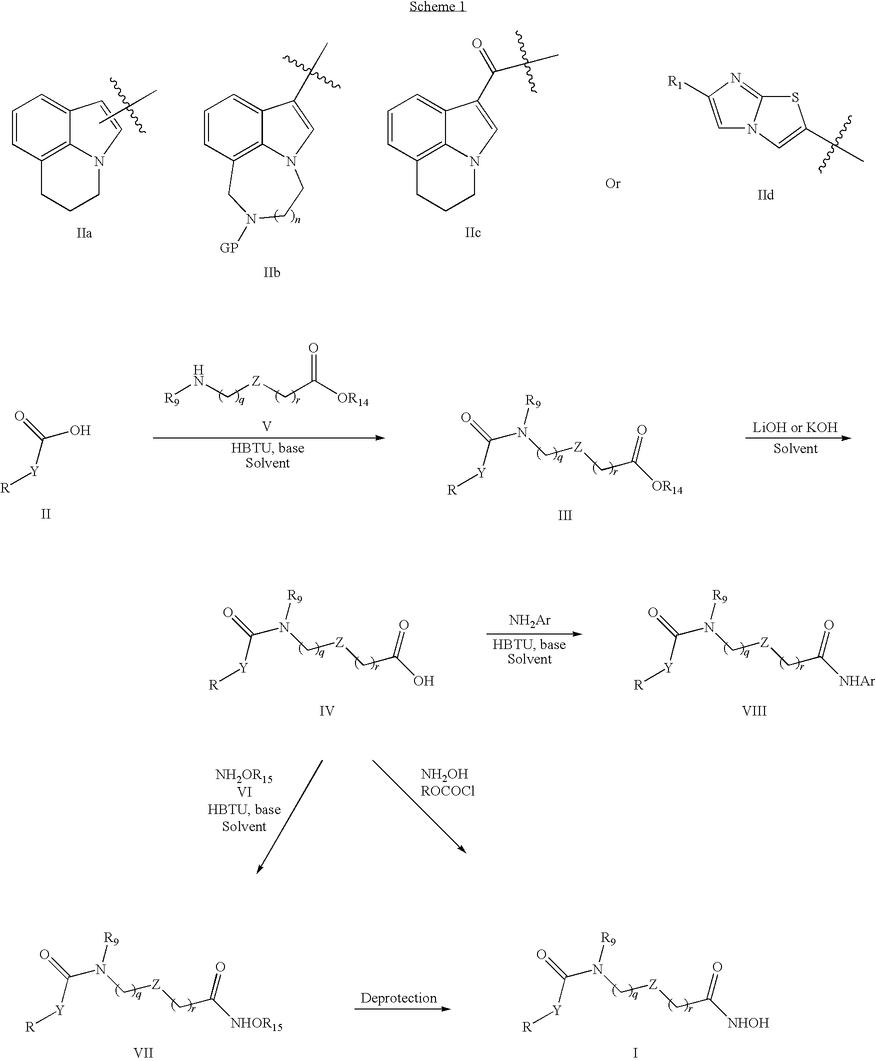

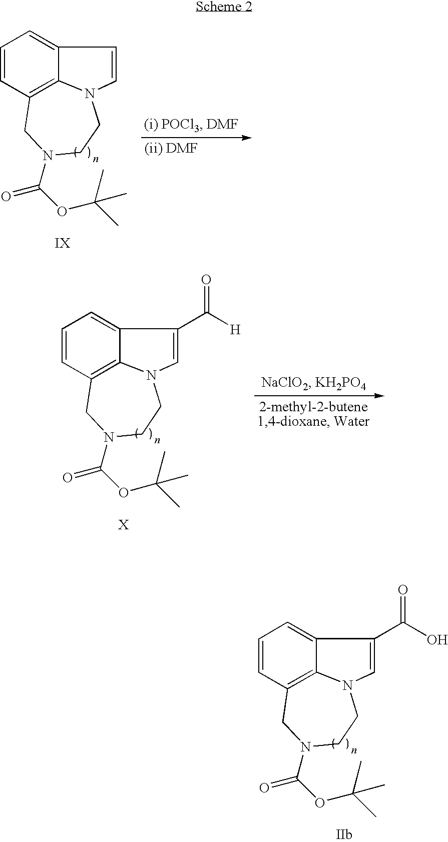

Compounds of formula I can be conveniently prepared by a variety of methods familiar to those skilled in the art. One common route is illustrated in Scheme 1. The tricycle acid II where R is IIa is readily prepared by methods described in the literature (WO 2006086484, EP 386628, DE 3907389) and known to those skilled in art. The tricycle acid II where R is IIb is readily prepared by methods described in the literature (WO 2003076442, WO 2001044247, Engler, Thomas et. al. J. Med. Chem., 2006, 47(16), 3934) and known to those skilled in art and by following method as shown in Scheme 2.

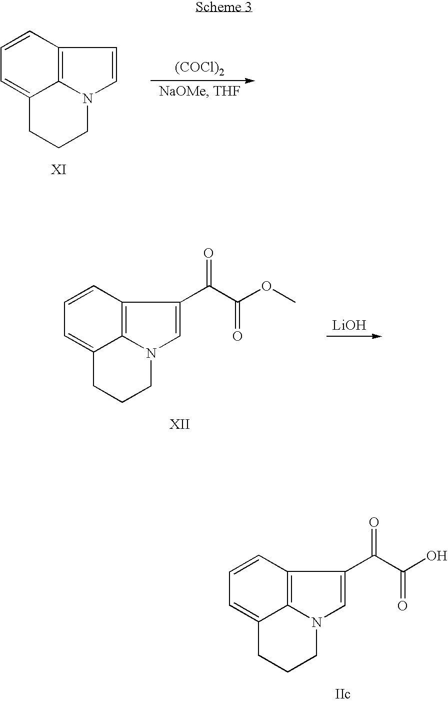

Tricyclic acid II where R is tricyclic keto IIc, it is prepared by methods described in the literature and known to those skilled in the art (WO 2006086484, Diana, P. et. al. Bioorganic & Medicinal Chemistry Letters, 2007, 17(8), 2342) and Scheme 3.

Acid II where R is imidazothiazole IId, it is prepared by methods described in the literature and known to those skilled in the art (Rubin Zhao et. al. Tetrahedron Letters, 2001, 2101 and WO 2004110990).

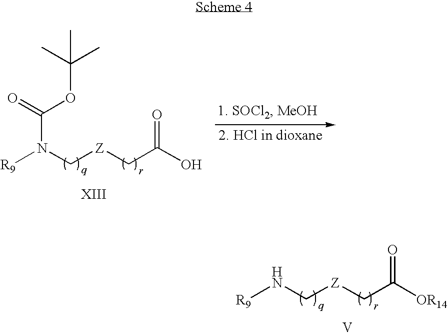

Ester V where R14 can be methyl, ethyl is prepared by treating t-BOC protected amino acids XIII with thionyl chloride in methanol (Scheme 4, Salauen A et. al., Journal of Organic Chemistry, 2006, 71(1), 150; Charvat T. et. al. Bioorganic Medicinal Chemistry, 2006, 14(13), 4552). Many amino acids are commercially available or readily prepared by methods described in the literature and known to those skilled in art.

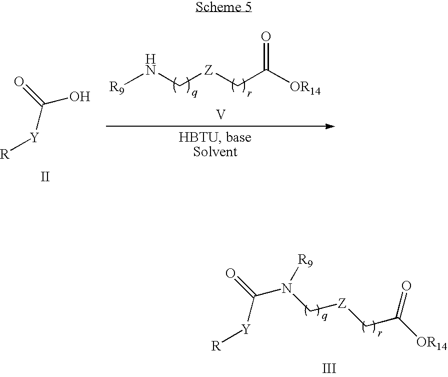

The carboxylic acid II is treated with ester V in presence of bases such as triethylamine or N,N-diisopropylethylamine and HBTU (O-(benzotriazo-1-yl)-N,N,N′,N′-tetramethyluronium hexafluorophosphate) in solvents such as N,N-dimethylformamide at room temperature (Kadzimirzis D. et.al., WO 2007059921; Boeglin D. et.al., Journal of the Medicinal Chemistry, 2007, 50(6), 1401; Johnson e. et.al., Tetrahedron Letters, 2007, 48(10), 1795) as shown in Scheme 5.

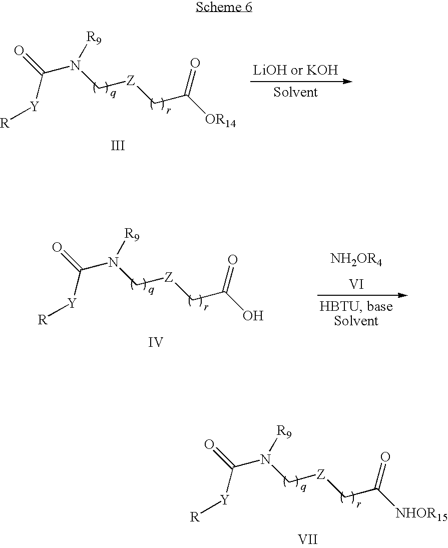

The carboxylic acid II are used to prepare protected hydroxamides of formula VII. These can be conveniently prepared by methods familiar to those skilled in the art (Scheme 6). The esters III are used to prepare carboxylic acid compounds of formula IV. These can be conveniently prepared by a variety of methods familiar to those skilled in the art. The ester III is treated with a aqueous solution of base such as lithium or potassium hydroxide in solvent mixture such as tetrahydrofuran/methanol for 0.5-4 hours at a room temperature to provide the acid IV. (Nicolaou, K. C. et. al., Angewandte Chemie, International Edition, 2006, 45(46), 7786; Organic Letters, 2006, 8(18), 4165). The carboxylic acid IV is treated with protected hydroxylamines VI, coupling agents such as HBTU, bases such as triethylamine and solvents such as N,N-dimethylformamide for 0.5-16 hours at ambient temperatures to provide the protected hydroxamides with formula VII. Alternatively tertiary amine bases such as N,N-diisopropylethylamine and solvents such as tetrahydrofuran can also be utilized.

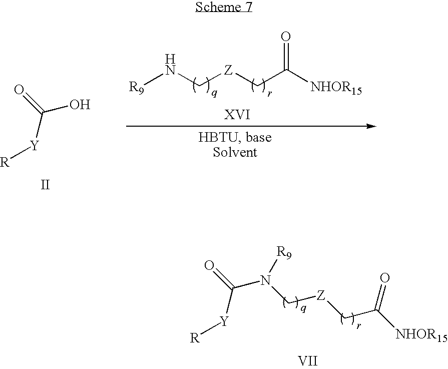

Alternatively, compound VII in present invention can also prepared from II and protected hydroxamide XVI as shown in scheme 7 and known to those skilled in the art.

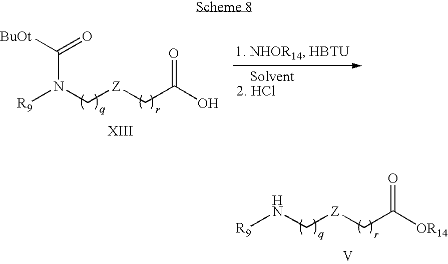

Compound V in the present invention can also be prepared as shown in scheme 7 and known to those skilled in the art.

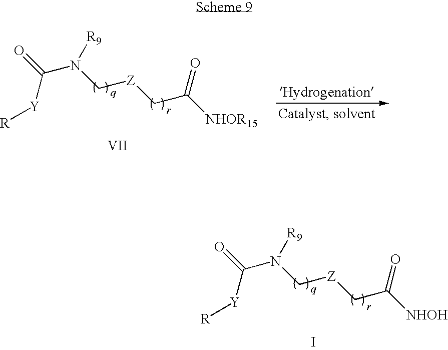

O-Protected hydroxamides VII are used to prepare the hydroxamic acid compounds with formula I. These can be conveniently prepared by methods familiar to those skilled in the art. Protected hydroxamides with formula VII where R₁₅ is benzyl, are treated with Pd (0) on carbon, in an atmosphere of hydrogen and with solvents such as methanol at ambient temperatures for 4-24 hours (Bioorganic Medicinal Chemistry, 2006, 14(21), 7241; 2006, 14(18), 6383; Journal of Medicinal Chemistry, 2005, 48(17), 5530). One common route is illustrated in Scheme 9.

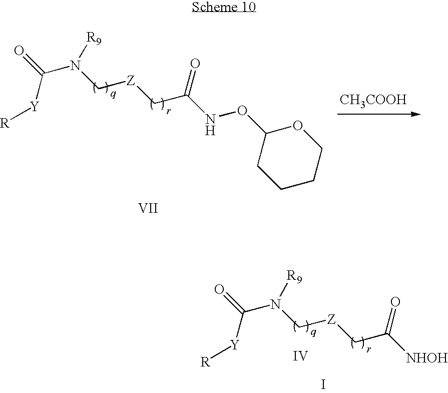

Alternatively, when R₁₅ is tetrahydropyranyl group as in the compounds of the formula VIIa, the protected hydroxamides are treated with acid such as acetic acid and solvents such as tetrahydrofuran and water in open air at 60° C. for 6-12 hours. Alternatively acids such as 10-camphorsulfonic acid, trifluoroacetic acid can also be used (Bioorganic Medicinal Chemistry, 2004, 12(16), 4351 and 2006, 14(22), 7625). One common route is illustrated in Scheme 10.

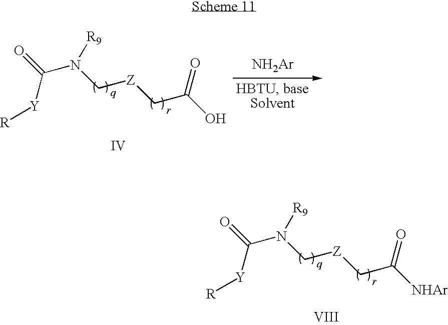

The compounds of the formula I in present invention where R₁₁ is NHAr can be prepared from the reaction of carboxylic acid II where R is IIa is prepared by methods described in literature and known to those skilled in the art. One common route is illustrated in Scheme 11 where acid IV is reacted with various substituted diamino benzenes to give the anilinamide derivatives VIII.

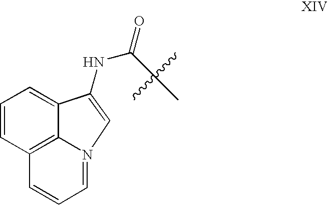

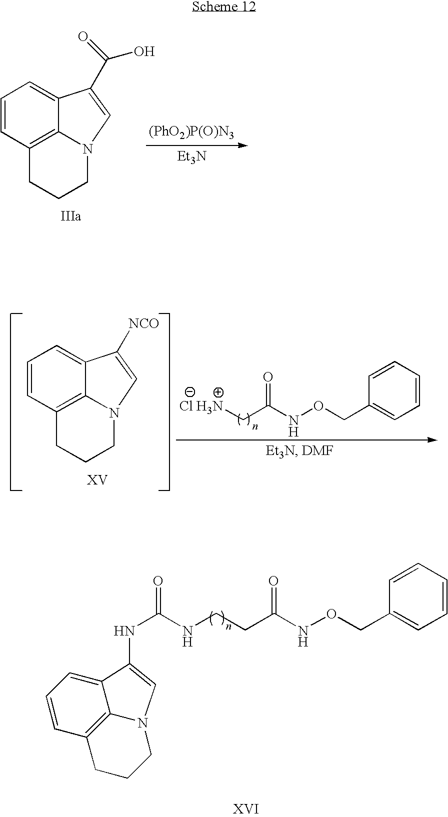

Tricyclic acid II where R is isocyanato tricycle XV, it is prepared by methods described in the literature (WO 2006086484; Nicolaou, K. C. et. al., Angewandte Chemie, International Edition, 2006, 45(46), 7786; Organic Letters, 2006, 8(18), 4165) and known to those skilled in the art and Scheme 12. Tricycle heterocycle XVI can be prepared by methods familiar to those skilled in the art. One common route is shown in Scheme 12.

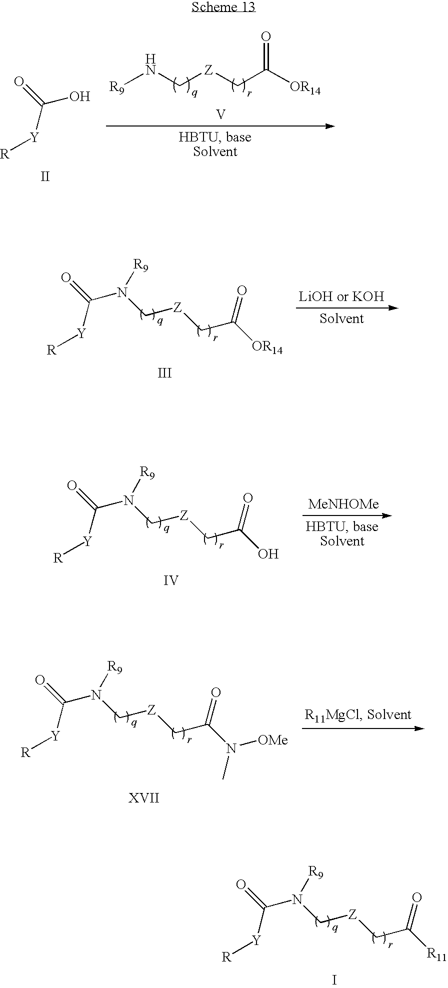

The compounds of the formula I in present invention where R₁₁ is an alkyl group can be prepared from the reaction of carboxylic acid IV with MeNHOMe by methods described in literature (Fuwa H. et. al. Bioorganic & Medicinal Chemistry Letters, 2006, 16(16), 4184; Albrecht S., Bioorganic & Medicinal Chemistry, 2006, 14(21), 7241) and known to those skilled in the art. One common route is illustrated in Scheme 13.

Compounds encompassed in the invention can be produced according to this or other synthetic processes without departing from the spirit or essential characteristics of the invention. All changes that come within the meaning and range of equivalency of the compounds are intended to be embraced herein. Thus, it is expected that one of ordinary skill in the art would know how to alter the synthetic schemes illustrated herein so as to produce a desired substitution pattern on a compound, produce an increased or decreased product yield, minimize reaction side products, eliminate the use of dangerous or toxic chemical reactants, and/or to produce a desired amount of product (e.g., scale-up reaction size for commercial manufacture), and the like.

The present invention further provides a compound prepared by one of the synthetic processes disclosed herein, such as those disclosed in the Examples.

### 3. Methods of Treatment

The present invention also provides a method for the treatment of a cell proliferative disorder in a mammal comprising administering to a mammal in need of such treatment, a therapeutically effective amount of a compound of Formula I. The invention further provides the use of a compound of Formula I for the ₚreparation of a medicament useful for the treatment of a cell proliferative disorder. In one embodiment, the invention provides for the treatment of cancer or precancerous conditions in a mammal comprising administering to a mammal in need of such treatment, a therapeutically effective amount of a compound of Formula I. The mammal can be e.g., any mammal, e.g., a human, a primate, mouse, rat, dog, cat, cow, horse, pig. For example, the mammal is a human.

An effective amount of a compound of Formula I is used in a method to treat a cell proliferative disorder in a mammal without affecting normal cells of the mammal For example, a therapeutically effective amount of a compound of Formula I is used in a method for treating cancer in a mammal by inducing cell death in cancer cells without affecting normal cells in the mammal Cell death can occur by either apoptosis or necrosis mechanisms. In another example, administration of a therapeutically effective amount of a compound of Formula I induces cell death in abnormally proliferating cells without inducing cell death in normal cells.

The invention also provides a method of protecting against a cell proliferative disorder in a mammal by administering a therapeutically effective amount of a compound of Formula I to a mammal The invention also provides the use of a compound of Formula I for the preparation of a medicament useful for the prevention of a cell proliferative disorder. In one embodiment, the invention provides for the prevention of cancer in a mammal comprising administering to a mammal in need of such treatment, a therapeutically effective amount of a compound of Formula I.

The compounds of the invention are administered in the form of pharmaceutical compositions, e.g., as described herein.

As used herein, a “subject” can be any mammal, e.g., a human, a primate, mouse, rat, dog, cat, cow, horse, pig, sheep, goat, camel. In a preferred aspect, the subject is a human.

As used herein, a “subject in need thereof” is a subject having a cell proliferative disorder, or a subject having an increased risk of developing a cell proliferative disorder relative to the population at large. In one aspect, a subject in need thereof has a precancerous condition. In a preferred aspect, a subject in need thereof has cancer.

As used herein, the term “cell proliferative disorder” refers to conditions in which the unregulated and/or abnormal growth of cells can lead to the development of an unwanted condition or disease, which can be cancerous or non-cancerous, for example a psoriatic condition. As used herein, the term “psoriatic condition” refers to disorders involving keratinocyte hyperproliferation, inflammatory cell infiltration, and cytokine alteration.

In one embodiment, the cell proliferation disorder is cancer. As used herein, the term “cancer” includes solid tumors, such as lung, breast, colon, ovarian, prostate, malignant melanoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS.

In addition to psoriatic conditions, the types of proliferative diseases which may be treated using the compositions of the present invention are epidermic and dermoid cysts, lipomas, adenomas, capillary and cutaneous hemangiomas, lymphangiomas, nevi lesions, teratomas, nephromas, myofibromatosis, osteoplastic tumors, and other dysplastic masses and the like. In one embodiment, proliferative diseases include dysplasias and disorders of the like.

As used herein, “monotherapy” refers to administration of a single active or therapeutic compound to a subject in need thereof. Preferably, monotherapy will involve administration of a therapeutically effective amount of an active compound. For example, cancer monotherapy with one of the compound of the present invention, or a pharmaceutically acceptable salt, prodrug, metabolite, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, montherapy with a compound of the present invention is more effective than combination therapy in inducing a desired biological effect.

As used herein, “treating” describes the management and care of a patient for the purpose of combating a disease, condition, or disorder and includes the administration of a compound of the present invention to prevent the onset of the symptoms or complications, alleviating the symptoms or complications, or eliminating the disease, condition or disorder.

In one aspect, treating cancer results in a reduction in size of a tumor. In another aspect, treating cancer results in a reduction in tumor volume. In another aspect, treating cancer results in a decrease in number of tumors. In another aspect, treating cancer results in a decrease in number of metastatic lesions in other tissues or organs distant from the primary tumor site. In another aspect, treating cancer results in an increase in average survival time of a population of treated subjects in comparison to a population receiving carrier alone. In another aspect, treating cancer results in an increase in average survival time of a population of treated subjects in comparison to a population of untreated subjects. In another aspect, treating cancer results in increase in average survival time of a population of treated subjects in comparison to a population receiving monotherapy with a drug that is not a compound of the present invention, or a pharmaceutically acceptable salt, prodrug, metabolite, analog or derivative thereof. In another aspect, treating cancer results in a decrease in the mortality rate of a population of treated subjects in comparison to a population receiving carrier alone. In another aspect, treating cancer results in a decrease in the mortality rate of a population of treated subjects in comparison to an untreated population. In a further aspect, treating cancer results a decrease in the mortality rate of a population of treated subjects in comparison to a population receiving monotherapy with a drug that is not a compound of the present invention, or a pharmaceutically acceptable salt, prodrug, metabolite, analog or derivative thereof. In another aspect, treating cancer results in a decrease in tumor growth rate. In another aspect, treating cancer results in a decrease in tumor regrowth.

In another aspect, treating or preventing a cell proliferative disorder results in a reduction in the rate of cellular proliferation. In another aspect, treating or preventing a cell proliferative disorder results in a reduction in the proportion of proliferating cells. In another aspect, treating or preventing a cell proliferative disorder results in a decrease in size of an area or zone of cellular proliferation. In another aspect, treating or preventing a cell proliferative disorder results in a decrease in the number or proportion of cells having an abnormal appearance or morphology.

In additional aspects, a compound of the present invention, or a pharmaceutically acceptable salt, metabolite, analog or derivative thereof, can be administered in combination with a chemotherapeutic agent. Exemplary chemotherapeutics with activity against cell proliferative disorders are known to those of ordinary skill in the art, and may be found in reference texts such as the Physician's Desk Reference, 59^{th} Edition, Thomson PDR (2005). For example, the chemotherapeutic agent can be a taxane, an aromatase inhibitor, an anthracycline, a microtubule targeting drug, a topoisomerase poison drug, a targeted monoclonal or polyconal antibody, an inhibitor of a molecular target or enzyme (e.g., a kinase inhibitor), or a cytidine analogue drug. In preferred aspects, the chemotherapeutic agent can be, but is not restricted to, tamoxifen, raloxifene, anastrozole, exemestane, letrozole, cisplatin, carboplatin, TAXOL® (paclitaxel), cyclophosphamide, lovastatin, minosine, GEMZAR® (gemcitabine HCl), araC, 5-fluorouracil (5-FU), methotrexate (MTX), TAXOTERE® (docetaxel), ZOLADEX® (goserelin), vincristin, vinblastin, nocodazole, teniposide, etoposide, epothilone, navelbine, camptothecin, daunonibicin, dactinomycin, mitoxantrone, amsacrine, doxorubicin (adriamycin), epirubicin, idarubicin, or GLEEVEC® (imatanib), IRESSA® (gefitinib), TARCEVA® (erlotinib), NEXAVAR® (sorafenib), SUTENT® (sunitinib malate), HERCEPTIN® (trastuzumab), RITUXAN® (Rituximab), ERBITUX® (cetuximab), AVASTIN® (bevacizumab), or agents listed in http://www.cancer.org/docroot/cdg/cdg_{—}0.asp. In another aspect, the chemotherapeutic agent can be a cytokine such as G-CSF (granulocyte colony stimulating factor). In another aspect, a compound of the present invention, or a pharmaceutically acceptable salt, metabolite, analog or derivative thereof may be administered in combination with radiation therapy. In yet another aspect, a compound of the present invention, or a pharmaceutically acceptable salt, metabolite, analog or derivative thereof may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), or CMFP (cyclophosphamide, methotrexate, 5-fluorouracil and prednisone).

Evolving understanding in the field of chromatin remodeling has changed the understanding of the ways in which genes are regulated. Interplay between histone deacetylases (HDACs) and histone acetyltransferases (HATs) increasingly reveals their involvement as players in regulating expression of neuronal tissue specific genes. It has been shown that genetic mutations encoding HDAC-binding proteins cause neurological disorders including Rett's syndrome and the mental retardation-associated Rubinstein-Taybi syndrome. Recently, HDAC inhibitors have been found to ameliorate progression of the spinal muscular atrophy (SMA), motor neuron disease and the Huntington's disease mouse models (Gray & Dangond F, Epigenetics, 1(2):67-75 (2006)). A potential therapeutic role for HDAC modulation may exist in Huntington's disease in that HDAC inhibitors have been shown to decrease the cognitive and motor effects associated with that syndrome (Bates, Nature, 413:691-694 (2001)). Studies have indicated that HDAC inhibitors may also diminish the progressive neurodegeneration associated with Parkinson's disease (PD) through the cytoplasmic sequesteration of α-synuclein (Kontopoulos et al, Human Molecular Genetics, 15:3012-3023 (2006)). Evidence indicates that even Alzhimer's disease may be moderated by HDAC inhibitors, by addressing the transcriptional dysregulation of proteins which modify amyloid precursor protein (APP) (Cao & Sudhof, Science, 293:115-120 (2001)). Overall, there is a viable rationale for leveraging the neuron-protective element of HDAC inhibitors in the treatment of human central nervous system (CNS) disorders.

The present invention also provides a method for the treatment of a central nervous system (CNS) disorder in a mammal comprising administering to a mammal in need of such treatment, a therapeutically effective amount of a compound of Formula I. The invention further provides the use of a compound of Formula I for the preparation of a medicament useful for the treatment of a human central nervous system (CNS) disorder. The mammal can be e.g., any mammal, e.g., a human, a primate, mouse, rat, dog, cat, cow, horse, pig. For example, the mammal is a human. In an embodiment, the human central nervous system (CNS) disorder is selcted from the group consisting of Rett's syndrome, the mental retardation-associated Rubinstein-Taybi syndrome, spinal muscular atrophy (SMA), motor neuron disease, Huntington's disease, Parkinson's disease (PD), and Alzhimer's disease.

### 4. The Pharmaceutical Compositions and Formulations

A “pharmaceutically acceptable salt” or “salt” of the disclosed compound is a product of the disclosed compound that contains an ionic bond, and is typically produced by reacting the disclosed compound with either an acid or a base, suitable for administering to a subject. Pharmaceutically acceptable salt can include, but is not limited to, acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na, K, Li, alkali earth metal salts such as Mg or Ca, or organic amine salts.

A “pharmaceutical composition” is a formulation containing the disclosed compounds in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salts thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

The present invention also provides pharmaceutical formulations comprising a compound of Formula I in combination with at least one pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, Pa., which is incorporated herein by reference. Examples of such carriers or diluents include, but are not limited to, water, saline, Ringer's solutions, dextrose solution, and 5% human serum in. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Methods for formulation are disclosed in PCT International Application PCT/US02/24262 (WO03/011224), U.S. Patent Application Publication No. 2003/0091639 and U.S. Patent Application Publication No. 2004/0071775, each of which is incorporated by reference herein.

A compound of Formula I is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of a compound of Formula I (as an active ingredient) with standard pharmaceutical carriers or diluents according to conventional procedures (i.e., by producing a pharmaceutical composition of the invention). These procedures may involve mixing, granulating, and compressing or dissolving the ingredients as appropriate to attain the desired preparation. In another embodiment, a therapeutically effective amount of a compound of Formula I is administered in a suitable dosage form without standard pharmaceutical carriers or diluents.

Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, ethylcellulose, hydroxypropylmethylcellulose, methylmethacrylate or the like. Other fillers, excipients, flavorants, and other additives such as are known in the art may also be included in a pharmaceutical composition according to this invention.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients and/or auxiliaries which facilitate processing of the active compounds into preparations that can be used pharmaceutically. Of course, the appropriate formulation is dependent upon the route of administration chosen.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. For treatment of psoriatic conditions, systemic administration (e.g., oral administration), or topical administration to affected areas of the skin, are preferred routes of administration. The dose chosen should be sufficient to constitute effective treatment but not so high as to cause unacceptable side effects. The state of the disease condition (e.g., cancer, psoriasis, and the like) and the health of the patient should be closely monitored during and for a reasonable period after treatment.

Representative compounds of the present invention are also shown in the Examples.

### Examples

Examples are provided below to further illustrate different features of the present invention. The examples also illustrate useful methodology for practicing the invention. These examples do not limit the claimed invention.

### Example 1

### Synthesis of methyl 5,6-dihydro-4H-pyrrolo[3,2,1-ij]quinolin-1-yl(oxo)acetate

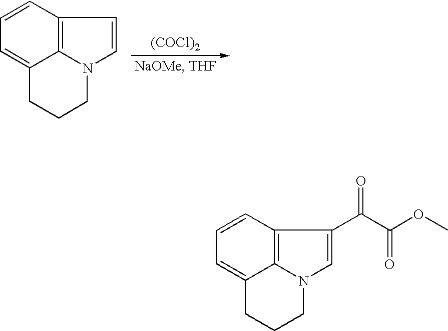

To a solution of 5,6,9a,9b-tetrahydro-4H-pyrrolo[3,2,1-ij]quinoline (5.0 g, 34.0 mmol) in anhydrous THF at 0° C. was added dropwise oxalyl chloride (17.0 mL, 34.0 mmol, 2.0M in dichloromethane). The mixture was stirred for 30 min and then cooled to −78° C. Sodium methoxide (75 mL, 150 mmol, 0.5M in methanol) was added slowly and the mixture was allowed to warm to room temperature over 2 hrs. The reaction mixture was diluted with ethyl acetate (300 mL), washed with water (250 mL), and brine (250 mL). The combined organic layers were dried over sodium sulfate and evaporated to dryness. The residue was filtered through a 5-inch plug of silica gel (50% EtOAc in hexanes) to give 87% (7.19 g) of as a yellow solid. M.p.=105-108° C.; 400 MHz ¹H NMR (CDCl₃) δ: 8.31 (s, 1H), 8.14 (d, J=7.8 Hz, 1H), 7.22 (t, J=7.4 Hz, 1H), 7.04 (d, J=7.0 Hz, 1H), 4.2 (t, J=5.4 Hz, 2H), 3.94 (s, 3H), 3.0 (t, J=5.8 Hz, 2H), 2.3 (t, J=5.8 Hz, 2H); LCMS: 244 [M+H].

### Example 2

### Synthesis of 5,6-dihydro-4H-pyrrolo[3,2,1-ij]quinolin-1-yl(oxo)acetic acid

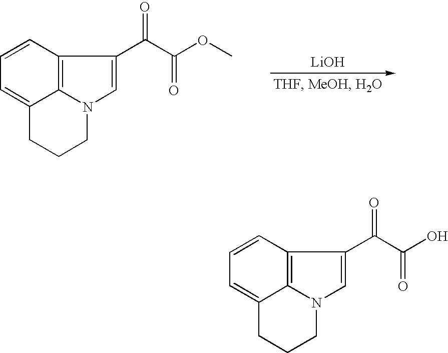

To a solution of methyl 5,6-dihydro-4H-pyrrolo[3,2,1-ij]quinolin-1-yl(oxo)acetate (1.0 g, 4.1 mmol) in THF (20 mL) and water (1 mL) was added lithium hydroxide (98 mg, 4.1 mmol). The reaction mixture was allowed to stir at room temperature for 18 h and then evaporated to dryness. The residue was dissolved in EtOAc:H₂O (1:1), acidified with 1M HCl (10 mL), and extracted with dichloromethane (5×50 mL). The combined organic layers were washed with brine (100 mL), dried over sodium sulfate, and the solvents were evaporated under reduced pressure to afford 87% (820 mg) of as a bright yellow solid. M.p.=152-155° C.; 400 MHz ¹H NMR (DMSO-d₆) 11.80 (brs, 1H), 8.42 (s, 1H), 7.90 (d, J=7.4 Hz, 1H), 7.21 (t, J=7.4 Hz, 1H), 7.04 (d, J=8.2 Hz, 1H), 4.29 (t, J=5.8 Hz, 2H), 2.95 (t, J=5.8 Hz, 2H), 2.15 (t, J=5.8 Hz, 2H); LCMS: 230 [M+H].

### Example 3

### Synthesis of 6-(3-methoxyphenyl)imidazo[2,1-b][1,3]thiazole-2-carboxlic acid

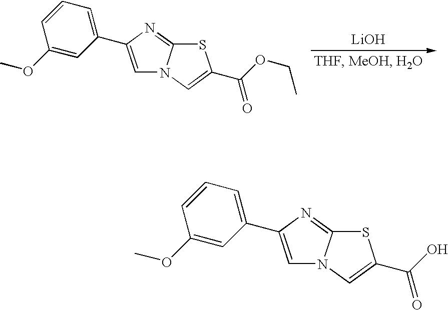

6-(3-methoxyphenyl)imidazo[2,1-b][1,3]thiazole-2-carboxylic acid was synthesized stirring ethyl 6-(3-methoxyphenyl)imidazo[2,1-b][1,3]thiazole-2-carboxylate (0.500 g, 1.65 mmol) in THF (10 mL) and methanol (3 mL) and an aqueous solution of LiOH (0.077 g, 1.83 mmol) in water (3 mL) at room temperature. Upon completion of reaction as observed via LCMS, was quenched with conc. HCl to pH 4 and extracted with dichloromethane (3×50 mL) and dried over anhydrous Na₂SO₄. The drying agent was filtered off and solvent removed under vacuo and residue obtained was used crude for the next step. LCMS: 275 (M+H]

### Example 4

### Synthesis of ethyl-7-N-benzylamino heptanoate

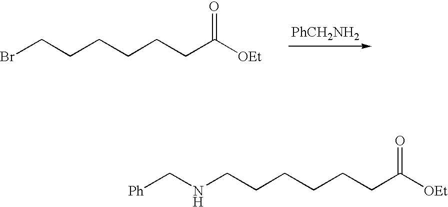

To a solution of ethyl 7-bromoheptanoate (1.0 g, 4.22 mmol) in THF (10 mL) was added benzylamine (2.3 mL, 21.08 mmol). The reaction was stirred at room temperature for 5 hours. The solvent was removed under reduced pressure. 20 mL of EtOAc was added to the residue. The solid was filtered out and the filtrate was concentrated to dryness. The crude product was ready for next step. LCMS: 264 [M+H].

### Example 5

### General Scheme 1

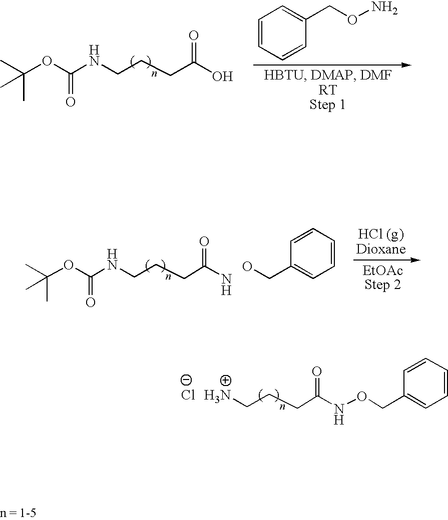

### Example 5.1

### General Procedure A: Step 1

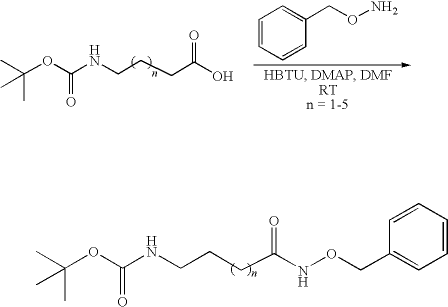

### Example 5.1.1

### Synthesis of tert-butyl{4-[(benzyloxy)amino]-4-oxobutyl}carbamate

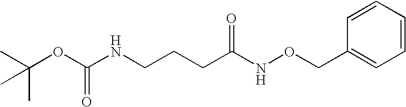

To a solution of the 4-[(tert-butoxycarbonyl)amino]butanoic acid (5.602 g, 27.6 mmol) in DMF (40 mL) was added HBTU (11.0 g, 29.0 mmol), triethylamine (5.81 mL, 41.45 mmol) and dimethlyaminopyridine (0.845 g, 6.9 mmol) followed by O-benzylhydroxylamine (4.19 g, 26.2 mmol). The reaction mixture was stirred for 16 hours. The reaction was quenched by adding water (200 mL). The aqueous layer was extracted with EtOAc (4×50 mL). The combined organic extract was washed with saturated 1.0 N HCl (2×100 mL), sodium bicarbonate (2×100 mL), water (2×100 mL), dried with sodium sulfate and the solvent removed under reduced pressure. The crude product was purified by flash column chromatography (SiO₂, 70% EtOAc in hexanes) to afford 5.41 g of pure final product as a light yellow solid. ¹H NMR (CDCl₃) 400 MHz δ 9.35 (s, 1H), 7.46-7.3 (m, 5H), 5.0-4.62 (m, 3H), 3.2-3.03 (m, 2H), 2.15-2.05 (m, 2H), 1.8-1.7 (m, 2H), 1.41 (s, 9H); LCMS=309 [M+H].

### Example 5.1.2

### Synthesis of tert-butyl{5-[(benzyloxy)amino]-5-oxopentyl}carbamate

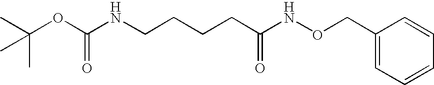

Compound tert-butyl{5-[(benzyloxy)amino]-5-oxopentyl}carbamate was synthesized using 5-[(tert butoxycarbonyl)amino]pentanoic acid and conditions outlined in Procedure A. M.p.=92-94° C.; ¹H NMR (CDCl₃) 400 MHz δ8.31 (s, 1H), 7.46-7.3 (m, 5H), 5.0-4.80 (m, 2H), 4.65-4.55 (m, 1H), 3.2-3.03 (m, 2H), 2.15-2.00 (m, 2H), 1.7-1.58 (m, 2H), 1.55-1.40 (m, 2H), 1.42 (s, 9H); LCMS=323 [M+H].

### Example 5.1.3

### Synthesis of tert-butyl{6-[(benzyloxy)amino]-6-oxohexyl}carbamate

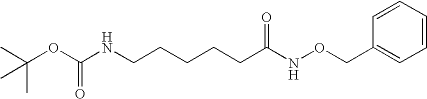

Compound tert-butyl{6-[(benzyloxy)amino]-6-oxohexyl}carbamate was synthesized using 6-[(tert-butoxycarbonyl)amino]hexanoic acid and conditions outlined in Procedure A. M.p.=52-54° C.; ¹H NMR (CDCl₃) 400 MHz δ 8.12 (s, 1H), 7.44-7.35 (m, 5H), 5.0-4.80 (m, 2H), 4.60-4.50 (m, 1H), 3.15-3.03 (m, 2H), 2.15-2.00 (m, 2H), 1.7-1.58 (m, 2H), 1.55-1.40 (m, 2H), 1.43 (s, 9H), 1.38-1.22 (m, 2H); LCMS=337 [M+H].

### Example 5.1.4

### Synthesis of tert-butyl{7-[(benzyloxy)amino]-7-oxoheptyl}carbamate

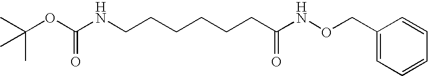

Compound tert-butyl{7-[(benzyloxy)amino]-7-oxoheptyl}carbamate was synthesized using 7-[(tert-butoxycarbonyl)amino]heptanoic acid and conditions outlined in Procedure A. M.p.=55-57° C.; ¹H NMR (CDCl₃) 400 MHz δ 8.29 (s, 1H), 7.44-7.35 (m, 5H), 5.0-4.80 (m, 2H), 4.60-4.55 (m, 1H), 3.15-3.03 (m, 2H), 2.15-2.00 (m, 2H), 1.65-1.58 (m, 2H), 1.55-1.40 (m, 2H), 1.43 (s, 9H), 1.38-1.22 (m, 4H); LCMS=351 [M+H].

### Example 5.1.5

### Synthesis of tert-butyl{8-[(benzyloxy)amino]-8-oxooctyl}carbamate

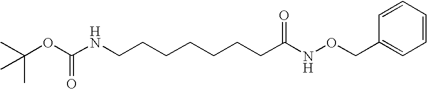

Compound tert-butyl{8-[(benzyloxy)amino]-8-oxooctyl}carbamate was synthesized using 8-[(tert-butoxycarbonyl)amino]octanoic acid and conditions outlined in Procedure A. M.p.=55-57° C.; ¹H NMR (CDCl₃) 400 MHz δ 8.19 (s, 1H), 7.44-7.35 (m, 5H), 5.0-4.80 (m, 2H), 4.60-4.50 (m, 1H), 3.15-3.03 (m, 2H), 2.15-2.00 (m, 2H), 1.65-1.58 (m, 2H), 1.55-1.40 (m, 2H), 1.43 (s, 9H), 1.38-1.22 (m, 6H); LCMS=365 [M+H].

### Example 5.2

### General Procedure B: Step 2

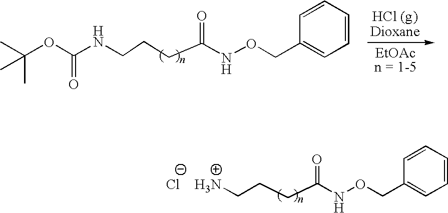

### Example 5.2.1

### Synthesis of 4-amino-N-(benzyloxy)butanamide hydrochloride

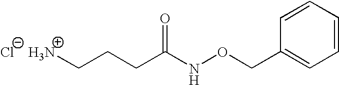

To a solution of tert-butyl{4-[(benzyloxy)amino]-4-oxobutyl}carbamate (5.41 g, 17.6 mmol) in EtOAc (100 mL) was added 4.0M solution of HCl (g) in 1,4-dioxane (20 mL), The reaction was allowed to stir at room temperature for 16 hours. The desired product separated as a white solid, which was filtered and dried under reduced pressure. M.p=77-78° C.; ¹H NMR (DMSO-d₆) 400 MHz δ 11.18 (s, 1H), 8.02 (s, 3H), 7.43-7.3 (m, 5H), 4.79 (s, 2H), 2.8-2.7 (m, 2H), 2.15-2.05 (m, 2H), 1.82-1.7 (m, 2H); LCMS−209 [M+H].

### Example 5.2.2

### Synthesis of 5-amino-N-(benzyloxy)pentanamide hydrochloride

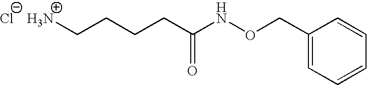

Compound 5-amino-N-(benzyloxy)pentanamide hydrochloride was synthesized using tert-butyl{5-[(benzyloxy)amino]-5-oxopentyl}carbamate and conditions outlined in Procedure B. M.p=129-132° C.; ¹H NMR (DMSO-d₆) 400 MHz δ 11.18 (s, 1H), 8.08 (s, 3H), 7.43-7.3 (m, 5H), 4.79 (s, 2H), 2.8-2.55 (m, 2H), 2.05-1.95 (m, 2H), 1.8-1.6 (m, 4H); LCMS=223 [M+H].

### Example 5.2.3

### Synthesis of 6-amino-N-(benzyloxy)hexanamide hydrochloride

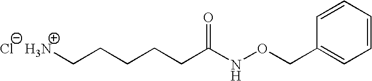

Compound 6-amino-N-(benzyloxy)hexanamide hydrochloride was synthesized using tert-butyl{6-[(benzyloxy)amino]-6-oxohexyl}carbamate and conditions outlined in Procedure B. M.p=86-88° C.; ¹H NMR (DMSO-d₆) 400 MHz δ 11.08 (s, 1H), 8.01 (s, 3H), 7.43-7.3 (m, 5H), 4.79 (s, 2H), 2.8-2.6 (m, 2H), 2.0-1.95 (m, 2H), 1.6-1.4 (m, 4H), 1.35-1.2 (m, 2H); LCMS=237 [M+H].

### Example 5.2.4

### Synthesis of 7-amino-N-(benzyloxy)heptanamide hydrochloride

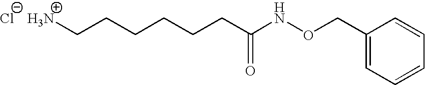

Compound 7-amino-N-(benzyloxy)heptanamide hydrochloride was synthesized using tert-butyl{7-[(benzyloxy)amino]-7-oxoheptyl}carbamate and conditions outlined in Procedure B. M.p=110-113° C.; ¹H NMR (DMSO-d₆) 400 MHz δ 11.05 (s, 1H), 7.97 (s, 3H), 7.43-7.3 (m, 5H), 4.78 (s, 2H), 2.8-2.6 (m, 2H), 2.0-1.95 (m, 2H), 1.6-1.4 (m, 4H), 1.38-1.2 (m, 4H); LCMS=251 [M+H].

### Example 5.2.5

### Synthesis of 8-amino-N-(benzyloxy)octanamide hydrochloride

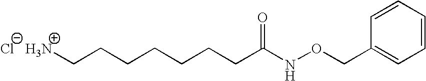

Compound 8-amino-N-(benzyloxy)octanamide hydrochloride was synthesized using tert-butyl{8-[(benzyloxy)amino]-8-oxooctyl}carbamate and conditions outlined in Procedure B. M.p=112-114° C.; ¹H NMR (DMSO-d₆) 400 MHz δ 11.05 (s, 1H), 7.97 (s, 3H), 7.43-7.3 (m, 5H), 4.78 (s, 2H), 2.8-2.6 (m, 2H), 2.0-1.95 (m, 2H), 1.6-1.4 (m, 4H), 1.38-1.2 (m, 6H); LCMS=265 [M+H].

### Example 6

### General Scheme 2

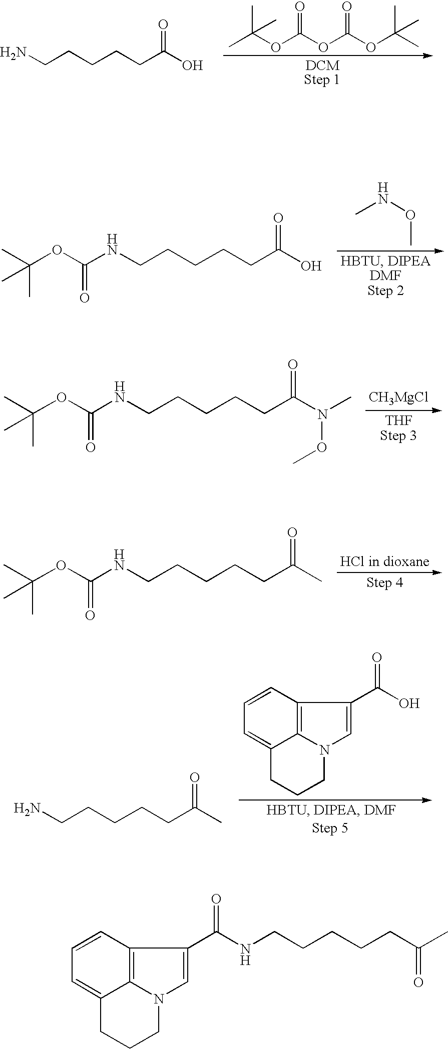

### Example 6.1

### Step 1: Synthesis of 6-[(tert-butoxycarbonyl)amino]hexanoic acid

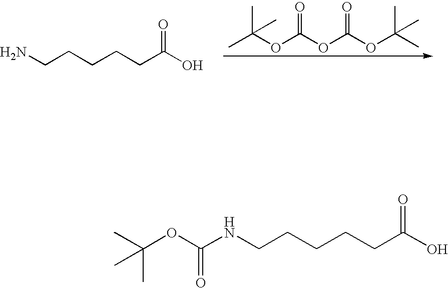

To a solution of 6-aminoheptanoic acid (2.0 g, 15.3 mmol) in dichloromethane (30 mL) was added di-tert-butyl dicarbonate (3.67 g, 16.8 mmol). The reaction was stirred at room temperature for 16 hours, filtered and the solvent removed under reduced pressure. The crude product was dried under high vacuum to afford 4.08 g (115%) of the desired product. 400 MHz ¹H NMR (DMSO-d₆) δ: 2.85 (q, J=13.2, 6.8 Hz, 2H), 2.15 (t, J=7.2 Hz, 2H), 1.47-1.43 (m, 4H), 1.34 (s, 9H), 1.22-1.18 (m, 2H). LCMS=232 [M+H].

### Example 6.2

### General Procedure C—Step 2

### Example 6.2.1

### Synthesis of tert-butyl{6-[methoxy(methyl)amino]-6-oxohexyl}carbamate

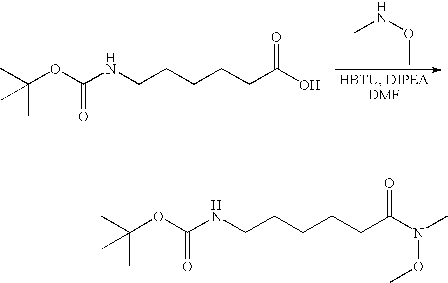

To a solution of 6-[(tert-butoxycarbonyl)amino]hexanoic acid (3.53 g, 15.27 mmol) in DMF (30 mL) was added Hunig's Base (8.03 mL, 47.34 mmol), HBTU (6.37 g, 16.8 mmol) followed by N,O-dimethylhydroxylamine (2.96 g, 30.54 mmol). The reaction mixture was stirred for 2 hours. The reaction mixture was diluted with diethyl ether (100 mL) and water (100 mL). The organic layer was separated and the aqueous layer extracted with diethyl ether (3×50 mL). The combined organic extracts was washed with brine (2×100 mL), dried with sodium sulfate and solvent removed under reduced pressure. The crude product was purified by flash column chromatography (SiO₂, 50% EtOAc in hexanes) to afford 3.84 g (97%) pure final product. 400 MHz ¹H NMR (DMSO-d₆) δ: 3.67 (s, 3H), 3.06 (s, 3H), 2.88 (q, J=12.8, 7.2 Hz, 2H), 2.34 (t, J=7.6 Hz, 2H), 1.49-1.42 (m, 4H), 1.37 (s, 9H), 1.24-1.18 (m, 2H). LCMS=275[M+H].

### Example 6.2.2

### Synthesis of tert-butyl 4-{3-[methoxy(methyl)amino]-3-oxopropyl}piperidine-1-carboxylate

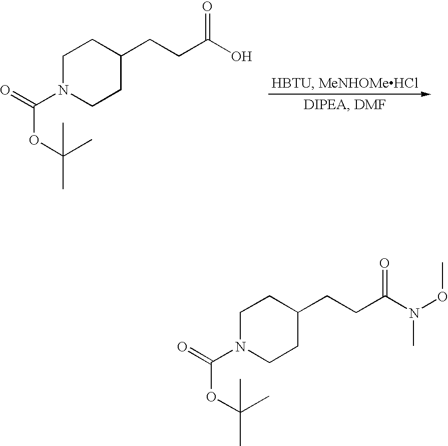

4-(2-carboxy-ethyl)-piperidine-1-carboxylic acid tert-butyl ester was reacted with O,N-dimethyl hydroxylamine using procedure C. Yield=97%. The crude material was used as is for the next step. LCMS: 301 [M+H].

### Example 6.3

### Step 3: Synthesis of tert-butyl(6-oxoheptyl)carbamate

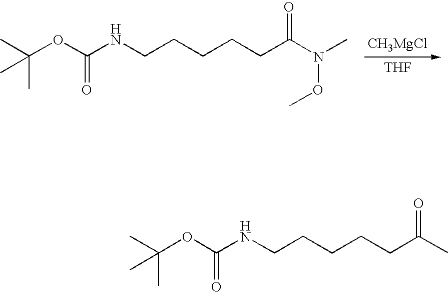

To a solution of tert-butyl{6-[methoxy(methyl)amino]-6-oxohexyl}carbamate (0.497 g, 1.8 mmol) in THF (8 mL) which had been cooled to −78° C. was added methyl magnesium bromide (1.2 g, 16.5 mmol) in a dropwise fashion. The reaction was stirred at −78° C. for 5 min and then allowed to warm to room temperature. The reaction mixture was stirred for 1 hour at room temperature and then quenched by adding saturated ammonium chloride (4 mL) and water (30 mL). The aqueous layer was extracted with dichloromethane (3×25 mL). The combined organic extract was washed with water (50 mL), dried with sodium sulfate and solvent removed under reduced pressure. The desired product 0.225 g (55%) was isolated as a light yellow oil and used in the next reaction without any purification. 400 MHz ¹H NMR (DMSO-d₆) δ: 2.87 (q, J=13.2, 6.8 Hz, 2H), 2.39 (t, J=7.2 Hz, 3H), 2.06 (s, 3H), 1.46-1.30 (m, 13H), 1.21-1.14 (m,2H).

### Example 6.4

### Step 4

### Example 6.4.1

### Synthesis of 7-aminoheptan-2-one

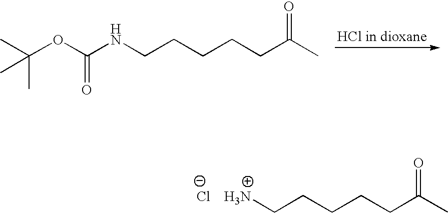

To a solution of the tert-butyl(6-oxoheptyl)carbamate (0.225 g) in 1,4-Dioxane (4 mL) was added 4M solution of HCl in 1,4-dioxane (2 mL). The reaction mixture was stirred for 16 hours. The solvent was removed under reduced pressure and the desired product 0.209 g (124%) was isolated as a yellow solid. The crude product was used in the next reaction without any further purification. LCMS=130 [M+H].

### Example 6.4.2

### Synthesis of 4-piperidin-4-ylbutan-2-one

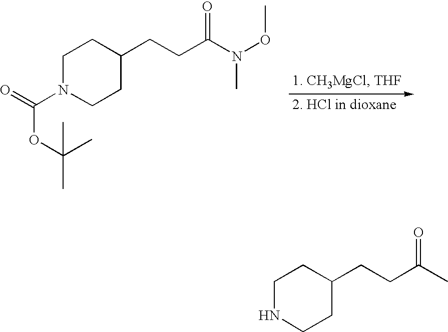

4-[Methoxy-methyl-carbamoyl)-ethyl]piperidine-1-carboxylic acid tert butyl ester (1.14 g, 3.38 mmol) was dissolved in anhydrous THF (10 ml) and cooled to 0° C. whereupon, methyl magnesium chloride (3M in THF, 6.76 ml, 20.3 mmol) was added and stirred at room temperature for 4 hours. Upon completion of reaction, saturated ammonium chloride (5 ml) was added followed by addition of water (20 ml) and stirred for 10 min. Additional water (10 ml) was added and extracted with dichloromethane (3×80 ml). The combined organic phase was dried over sodium sulfate and concentrated in vacuo, affording a clear oil (0.9 g, quant.). The crude material was used for the next step. LCMS: 256 [M+H]. 4-(3-Oxo-butyl)-piperidine-1-carboxylic acid tert-butyl ester (0.900 g, 3.53 mmol) was dissolved in 1,4-dioxane (4 ml) and 4 M solution of hydrogen chloride in 1,4-dioxane (2.0 ml, 20.3 mmol) was added and stirred at room temperature for 5 hours. Upon completion of reaction, the reaction mixture was concentrated in vacuo, affording the hydrochloride salt 4-piperidin-4-yl-butan-2-one (0.74 g, quant.). 400 MHz ¹H NMR (DMSO-d₆) δ: 3.87 (d, J=16.8 Hz, 2H), 2.60 (br s, 2H), 2.41 (t, J=5, 9.6 Hz, 2H), 2.04 (s, 3H), 1.56 (d, J=16.8 Hz, 2H), 1.26-1.38 (m, 5H), 0.86-0.94 (m, 2H). LCMS: 156 [M+H].

### Example 6.5

### General Procedure D—Step 5

### Example 6.5.1

### Synthesis of N-(6-oxoheptyl)-5,6-dihydro-4H-pyrrolo[3,2,1-ij]quinoline-1-carboxamide

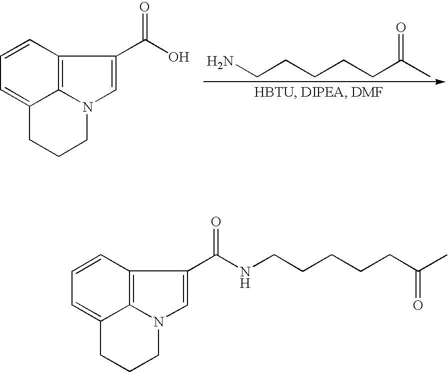

To a solution of 5,6-Dihydro-4-H-pyrrolo[3,2,1-ij]quinoline-1-carboxylic acid (0.201 g, 1 mmol) in DMF (8 ml) was added Hunig's Base (0.526 mL, 3.1 mmol) and HBTU (0.417 g, 1.1 mmol) followed by 7-aminoheptan-2-one hydrochloride (0.209 g, 1 mmol). The reaction mixture was stirred at room temperature for 4 hours. The reaction mixture was diluted with dichloromethane (100 mL) and water (100 mL). The organic layer was separated and the aqueous layer extracted with dichloroemthane (3×50 mL). The combined organic extracts was washed with brine (2×100 mL), dried with sodium sulfate and solvent removed under reduced pressure. The crude product was purified by flash column chromatography (SiO₂, 100% EtOAc to 2% methanol in dichloromethane) to afford 0.0368 g (12%) pure final product. 400 MHz ¹H NMR (DMSO-d₆) δ: 7.93 (s, 1H), 7.82 (d, J=7.6 Hz, 1H), 7.76-7.74 (m, 1H), 7.015 (t, J=7.6 Hz, 1H), 6.90 (d, J=6.4 Hz, 1H), 4.18 (t, J=5.6 Hz, 2H), 3.24-3.21 (m, 2H), 2.92 (t, J=5.6 Hz, 2H), 2.42 (t, J=7.6 Hz, 2H), 2.14-2.13 (m, 2H), 2.06 (s, 3H), 1.51-1.46 (m, 4H), 1.28-1.25 9m, 2H). LCMS: 313[M+H].

### Example 6.5.2

### Synthesis of 4-[1-(5,6-dihydro-4H-pyrrolo[3,2,1-ij]quinolin-1-ylcarbonyl)piperidin-4-yl]butan-2-one

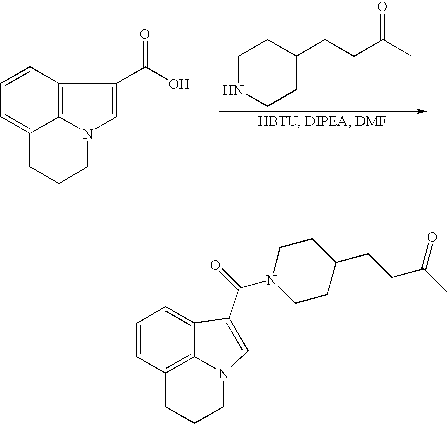

5,6-Dihydro-4-H-pyrrolo[3,2,1-ij]quinoline-1-carboxylic acid was reacted with 4-piperidin-4-ylbutan-2-one using procedure D. 400 MHz ¹H NMR (DMSO-d₆) δ: 7.66 (s, 1H), 7.40 (d, J=8 Hz, 1H), 7.01 (t, J=7.2 Hz, 1H), 6.90 (d, J=6.8 Hz, 1H), 4.26 (d, J=13.2 Hz, 2H), 4.19 (t, J=5.8 Hz, 2H), 2.89 (m, 4H), 2.46 (d, J=7.2 Hz, 2H), 2.14 (t, J=5.6 Hz, 2H), 1.67 (d, J=13.2 Hz, 2H), 1.45-1.41 (m, 2H), 1.085-1.049 (m, 2H). LCMS: 339[M+H].

### Example 7

### General Scheme 3

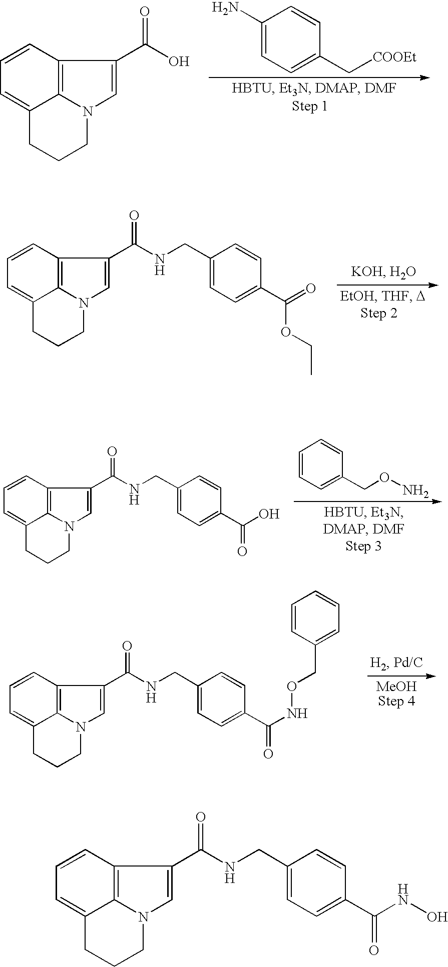

### Example 7.1

### General Procedure E—Step 1

### Example 7.1.1

### Synthesis of methyl 4-{[(5,6-dihydro-4H-pyrrolo[3,2,1-ij]quinolin-1-ylcarbonyl)amino]methyl}benzoate

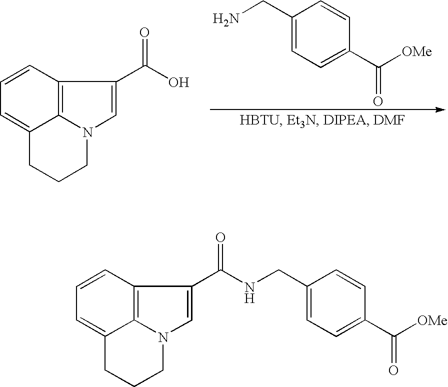

The 5,6-dihydro-4H-pyrrolo[3,2,1-ij]quinoline-1-carboxylic acid (0.5 g, 2.48 mmol) in DMF (30 ml) was treated with HBTU (0.95 g, 2.48 mmol). The mixture was kept at room temperature for 30 min. then the methyl 4-aminomethylbenzoate (0.552 g, 2.73 mmol) was added followed by DMAP (0.335 g, 2.73 mmol) and triethylamine (770 μl, 5.47 mmol). The reaction mixture was stirred at room temperature for 4 hours then poured into water (300 ml). The material was extracted with ethyl acetate (3×200 ml). Combined extracts were washed with water (100 ml) and a saturated aqueous solution of sodium chloride (100 ml). The organic phase was dried over sodium sulfate the concentrated in vacuo, affording a yellow solid. The crude material was triturated in Et₂O. The methyl 4-{[(5,6-dihydro-4H-pyrrolo[3,2,1-ij]quinolin-1-ylcarbonyl)amino]methyl}benzoate was obtained as a pale yellow solid (0.616 g, 71%). M.p.=197-198° C. 400 MHz ¹H NMR (DMSO-d₆) δ: 8.48 (t, J=6.0 Hz, 1H), 8.03 (s, 1H), 7.92 (d, J=8.7 Hz, 2H), 7.86 (d, J=8.1 Hz, 1H), 7.47 (d, J=8.4 Hz, 2H), 7.04 (t, J=7.5 Hz, 1H), 6.92 (d, J=7.3 Hz, 1H), 4.55 (d, J=5.9 Hz, 2H), 4.21 (t, J=5.9 Hz, 2H), 3.84 (s, 3H), 2.94 (t, J=5.9 Hz, 2H), 2.14 (quintuplet, J=5.7 Hz, 2H). LCMS: 349 [M+H].

### Example 7.1.2

### Synthesis of ethyl{4-[(5,6-dihydro-4H-pyrrolo[3,2,1-ij]quinolin-1-ylcarbonyl)amino]phenyl}acetate

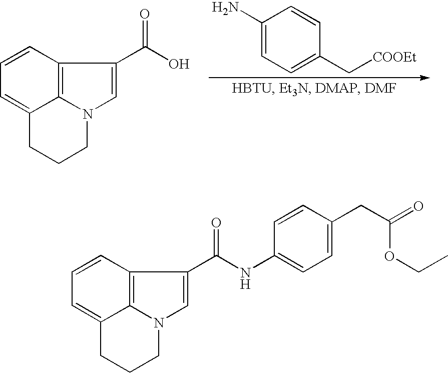

Ethyl 4-aminophenylacetate was reacted with 5,6-Dihydro-4-H-pyrrolo[3,2,1-ij]quinoline-1-carboxylic acid using procedure E. Yield=55%. 400 MHz ¹H NMR (CDCl₃) δ: 8.03 (s, 1H), 7.76 (d, J=8.4 Hz, 1H), 7.69 (s, 1H), 7.63 (d, J=8.4 Hz, 2H), 7.22 (d, J=8 Hz, 2H), 7.15 (t, J=7.6 Hz, 1H), 6.95 (d, J=6.8 Hz, 1H), 4.13 (q, J=7.2 Hz, 2H), 3.98 (t, J=5.6 Hz, 2H), 3.56 (s, 2H), 2.92 (t, J=6 Hz, 2H), 2.16-2.09 (m, 2H), 1.24 (t, J=7.2 Hz, 3H); LCMS: 363 [M+H]

### Example 7.1.3

### Synthesis of N-{5-[(benzyloxy)amino]-5-oxopentyl}-5,6-dihydro-4H-pyrrolo[3,2,1-ij]quinoline-1-carboxamide

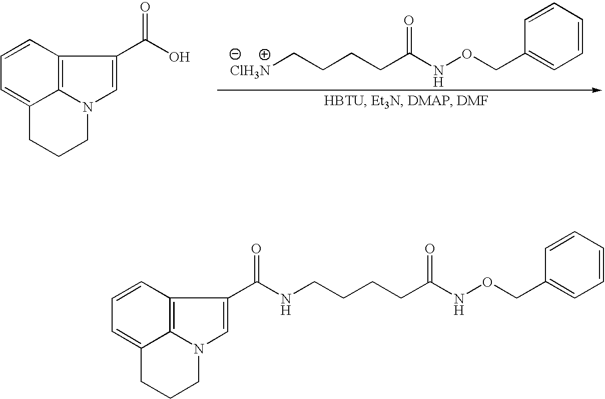

5,6-dihydro-4H-pyrrolo[3,2,1-ij]quinoline-1-carboxylic acid was reacted with 5-amino-pentanoic acid benzyloxy-amide hydrochloride using procedure E. Yield=38%; M.p. 154-155° C., 400 MHz ¹H NMR (DMSO-d₆) δ: 10.97 (s, 1H), 7.94 (s, 1H), 7.78 (m, 2H), 7.37-7.34 (m, 5H), 7.02 (t, J=7.6 Hz, 1H), 6.90 (d, J=6.80 Hz, 1H), 4.77 (s, 2H), 4.19 (t, J=5.60 Hz, 2H), 3.23 (m, 2H), 2.93 (t, J=5.80 Hz, 2H), 2.13 (m, 2H), 1.99 (t, J=7.0 Hz, 2H), 1.55-1.47 (m, 4H). LCMS: 406 [M+H].

### Example 7.1.4

### Synthesis of methyl 6-[(5,6-dihydro-4H-pyrrolo[3,2,1-ij]quinolin-1-ylcarbonyl)amino]hexanoate

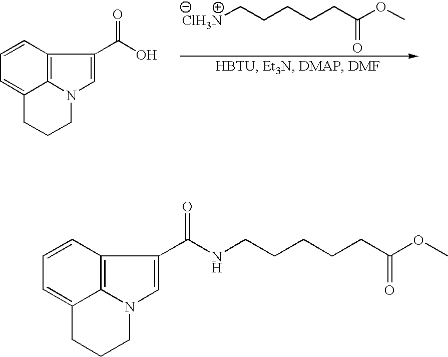

6-Dihydro-4-H-pyrrolo[3,2,1-ij]quinoline-1-carboxylic acid and methyl-7-amino hexanoate hydrochloride were reacted using procedure E. Yield=40%. 400 MHz ¹H NMR (DMSO-d₆) δ: 7.72 (s, 1H), 7.61 (d, J=8 Hz, 1H), 7.16 (d, J=7.2 Hz, 1H), 6.99 (d, J=6.82 Hz, 1H), 6.00 (m, 1H), 4.17 (t, J=5.47 Hz, 2H), 3.50 (q, J=12, 7.2 Hz, 2H), 3.00 (t, J=6 Hz, 2H), 2.33 (t, J=7.2 Hz, 2H), 2.26-2.20 (m, 2H), 1.73-1.63 (m, 4H), 1.48-1.41 (m, 2H). LCMS: 329[M+H].

### Example 7.1.5

### Synthesis of methyl 7-[(5,6-dihydro-4H-pyrrolo[3,2,1-ij]quinolin-1-ylcarbonyl)amino]heptanoate

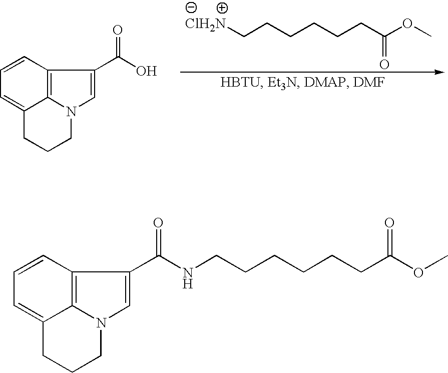

5,6-dihydro-4H-pyrrolo[3,2,1-ij]quinoline-1-carboxylic acid was reacted with methyl 7-aminoheptanoate hydrochloride salt using procedure E. Yield=48% M.p.=66-67° C.; 400 MHz ¹H NMR (CDCl₃) δ: 7.71 (s, 1H), 7.59 (d, J=8.0 Hz, 1H), 7.17-7.14 (m, 1H), 6.98 (d, J=6.8 Hz, 1H), 5.96 (bs, 1H), 4.17 (t, J=6.0 Hz, 2H), 3.66 (s, 3H), 3.50-3.46 (m, 2H), 3.00 (t, J=5.6 Hz, 2H), 2.31 (t, J=7.2 Hz, 2H), 2.23 (t, J=5.2 Hz, 2H), 1.71-1.63 (m, 4H), 1.50-1.38 (m, 4H); LCMS: 343 [M+H].

### Example 7.1.6

### Synthesis of methyl 7-[(5,6-dihydro-4H-pyrrolo[3,2,1-ij]quinolin-2-ylcarbonyl)amino]heptanoate

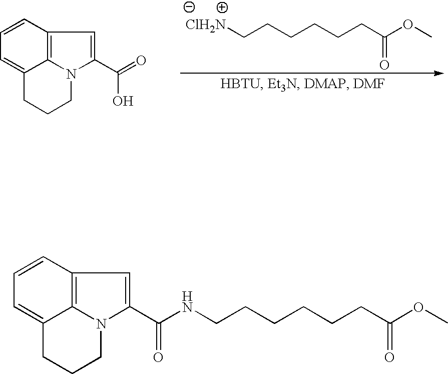

5,6-dihydro-4H-pyrrolo[3,2,1-ij]quinoline-2-carboxylic acid was reacted with methyl 7-aminoheptanoate hydrochloride salt using procedure E. LCMS: 343 [M+H].

### Example 7.1.7

### Synthesis of methyl 7-({[6-(3-methoxyphenyl)imidazo[2,1-b][1,3]thiazol-2-yl]carbonyl}amino)heptanoate

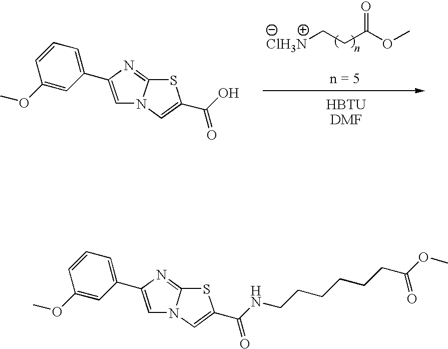

6-(3-methoxyphenyl)imidazo[2,1-b][1,3]thiazole-2-carboxylic acid was reacted with 7-aminoheptanoate hydrochloride salt following procedure E. LCMS: 416 [M+H].

### Example 7.1.8

### Synthesis of N-{8-[(benzyloxy)amino]-8-oxooctyl}-5,6-dihydro-4H-pyrrolo[3,2,1-ij]quinoline-1-carboxamide

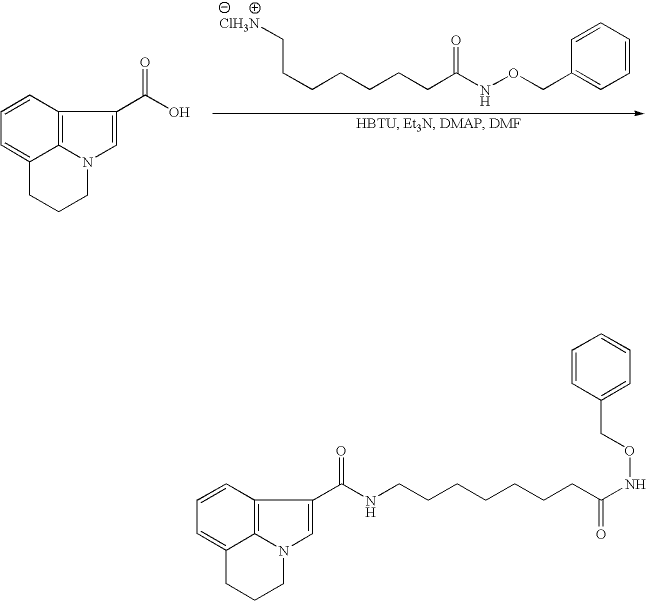

5,6-dihydro-4H-pyrrolo[3,2,1-ij]quinoline-1-carboxylic acid was reacted with 8-amino-N-(benzyloxy)octanamide hydrochloride using procedure E. Yield=82%. LCMS: 448 [M+H].

### Example 7.1.9

### Synthesis of methyl 3-[1-(5,6-dihydro-4H-pyrrolo[3,2,1-ij]quinolin-1-ylcarbonyl)piperidin-4-yl]propanoate

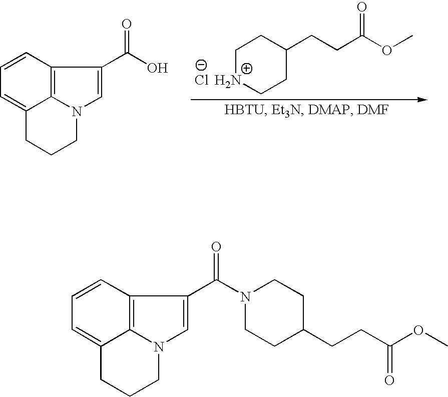

5,6-Dihydro-4-H-pyrrolo[3,2,1-ij]quinoline-1-carboxylic acid was reacted with methyl 3-piperidin-4-ylpropanoate hydrochloride following procedure E. Yield=38%. 400 MHz ¹H NMR (DMSO_{d6}) δ: 7.66 (s, 1H), 7.40 (d, J=8 Hz, 1H), 7.02 (td, J=7.2, 1.2 Hz, 1H), 6.91 (dd, J=6.8, 0.8 Hz, 1H), 4.27 (d, J=12.8 Hz, 2H), 4.19 (t, J=5.6 Hz, 2H), 3.59 9s, 3H), 2.96-2.83 (m, 4H), 2.35 (t, J=7.2 Hz, 2H), 2.14 (pent, J=5.6 Hz, 2H), 1.69 (d, J=11.6 Hz, 2H), 1.51 (t, J=6.0 Hz, 3H), 1.16-1.04 (m, 2H); LCMS: 355 [M+H].

### Example 7.1.10

### Synthesis of N-(benzyloxy)-4-{[5,6-dihydro-4H-pyrrolo[3,2,1-ij]quinolin-1-yl(oxo)acetyl]amino}butanamide

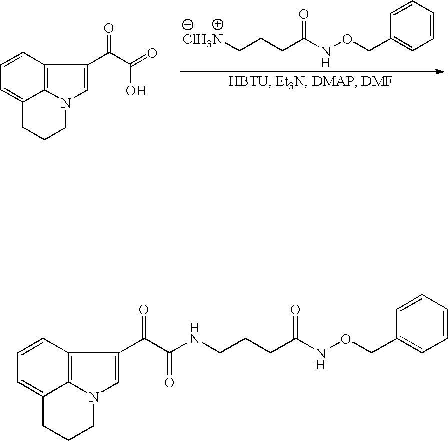

5,6-dihydro-4H-pyrrolo[3,2,1-ij]quinolin-1-yl(oxo)acetic acid was reacted with 4-amino-N-(benzyloxy)butanamide hydrochloride using procedure E. Yield=73%. M.p.=88-90° C.; 300 MHz ¹H NMR (DMSO-d₆) δ: 10.98 (s, 1H), 8.73 (s, 2H), 7.95 (d, J=7.9 Hz, 1H), 7.38 (bs, 5H), 7.18 (t, J=6.8 Hz, 1H), 7.05 (d, J=6.9 Hz, 1H), 4.78 (s, 2H), 4.29 (t, J=5.7 Hz, 2H), 3.20-3.17 (m, 2H), 2.95-2.92 (m, 2H), 2.18-2.11 (m, 2H), 2.01 (t, J=6.0 Hz, 2H), 1.74-1.71 (m, 2H); LCMS: 420 [M+H].

### Example 7.1.11

### Synthesis of N-(benzyloxy)-5-{[5,6-dihydro-4H-pyrrolo[3,2,1-ij]quinolin-1-yl(oxo)acetyl]amino}pentanamide

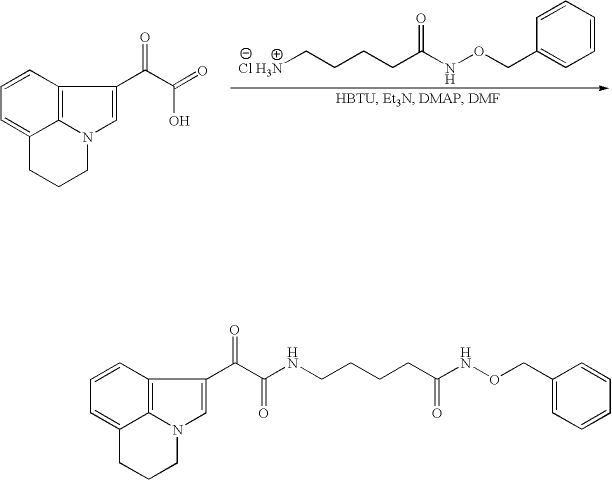

5,6-dihydro-4H-pyrrolo[3,2,1-ij]quinolin-1-yl(oxo)acetic acid was reacted with 5-amino-N-(benzyloxy)pentanamide hydrochloride using procedure E. Yield=69%. M.p.=62-64° C.; 3 MHz ¹HNMR (CDCl₃) δ: 10.97 (s, 1H), 8.73 (s, 2H), 7.95 (d, J=7.5 Hz, 1H), 7.37 (bs, 5H), 7.21 (t, J=7.5 Hz, 1H), 7.06 (d, J=6.9 Hz, 1H), 4.78 (s, 2H), 4.28 (t, J=5.4 Hz, 2H), 3.21-3.17 (m, 2H), 2.98-2.92 (m, 2H), 2.18-2.11 (m, 2H), 2.01 (t, J=6.0 Hz, 2H), 1.50 (bs, 4H); LCMS: 434 [M+H].

### Example 7.1.12

### Synthesis of N-(benzyloxy)-6-{[5,6-dihydro-4H-pyrrolo[3,2,1-ij]quinolin-1-yl(oxo)acetyl]amino}hexanamide

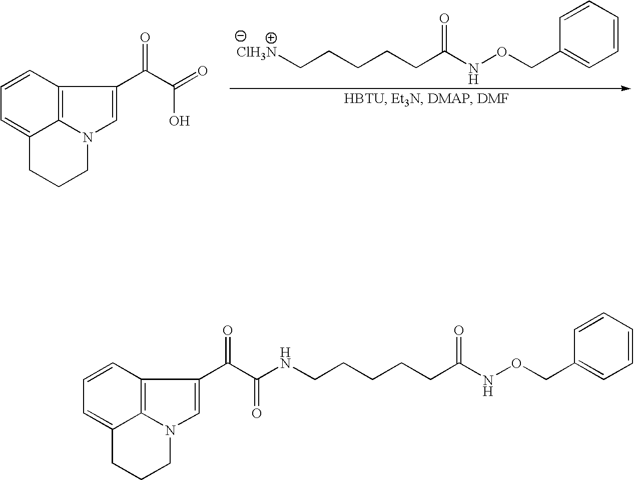

5,6-dihydro-4H-pyrrolo[3,2,1-ij]quinolin-1-yl)-oxo-acetic acid was reacted with 6-amino-N-(benzyloxy)hexanamide hydrochloride salt using procedure E. M.p.=119-121° C.; 400 MHz ¹H NMR (CDCl₃) δ: 8.92 (s, 1H), 8.21 (bs, 1H), 8.12 (d, J=8.0 Hz, 1H), 7.57 (bs, 1H), 7.36 (bs, 3H), 7.26-7.23 (m, 1H), 7.05 (d, J=6.8 Hz, 1H), 4.91 (bs, 2H), 4.19 (bs, 2H), 3.38-3.33 (m, 2H), 3.00 (t, J=6.0 Hz, 2H), 2.35 (bs, 1H), 2.25 (t, J=5.2 Hz, 2H), 2.05 (bs, 1H), 1.367-1.57 (m. 4H), 1.40-1.36 (m, 2H); LCMS: 448 [M+H].

### Example 7.1.13

### Synthesis of methyl 7-{[5,6-dihydro-4H-pyrrolo[3,2,1-ij]quinolin-1-yl(oxo)acetyl]amino}heptanoate

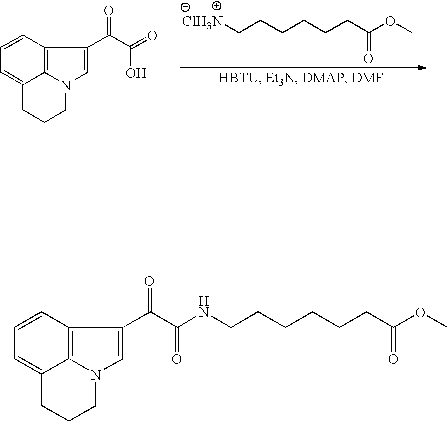

5,6-dihydro-4H-pyrrolo[3,2,1-ij]quinolin-1-yl oxoacetic acid was reacted with methyl 7-aminoheptanoate hydrochloride following procedure E. Yield=80% 400 MHz ¹H NMR (CDCl₃) δ: 8.94(s, 1H), 8.14 (d, J=7.8 Hz, 1H), 7.55 (brs, 1H), 7.27 (t, J=8.4 Hz, 1H), 7.07 (d, J=8.4 Hz, 1H), 4.23 (t, J=5.8 Hz, 2H), 3.67 (s, 3H), 3.4(m, 2H), 2.3 (t, J=5.8 Hz, 2H), 2.31 (m, 4H), 1.61 (m, 4H), 1.38 (m, 4H); LCMS: 371 [M+H].

### Example 7.1.14

### Synthesis of ethyl 7-[benzyl(5,6-dihydro-4H-pyrrolo[3,2,1-ij]quinolin-1-ylcarbonyl)amino]heptanoate

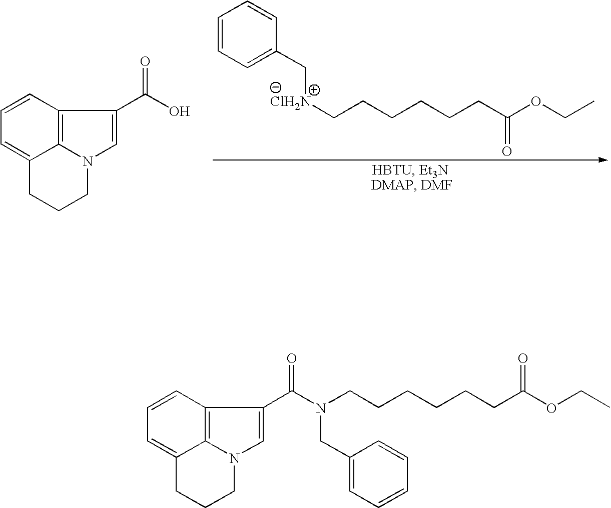

5,6-dihydro-4H-pyrrolo[3,2,1-ij]quinoline-1-carboxylic acid was reacted with ethyl 7-(benzylamino)heptanoate hydrochloride salt using procedure E. Yield=61%. 300 MHz ¹H NMR (CDCl₃) δ: 7.58 (s, J=8.1 Hz, 1H), 7.34-7.25 (m, 6H), 7.11 (t, J=7.2 Hz, 1H), 7.95 (d, J=6.9 Hz, 1H), 4.82 (s, 2H), 4.14-4.09 (m, 4H), 3.47 (t, J=7.2 Hz, 2H), 2.99 (t, J=6.3 Hz, 2H), 2.26-2.20 (m, 4H), 1.67-1.57 (m, 4H), 1.26-1.21 (m, 6H); LCMS: 447 [M+H].

### Example 7.1.15

### Synthesis of N-{7-[methoxy(methyl)amino]-7-oxoheptyl}-5,6-dihydro-4H-pyrrolo[3,2,1-ij]quinoline-1-carboxamide

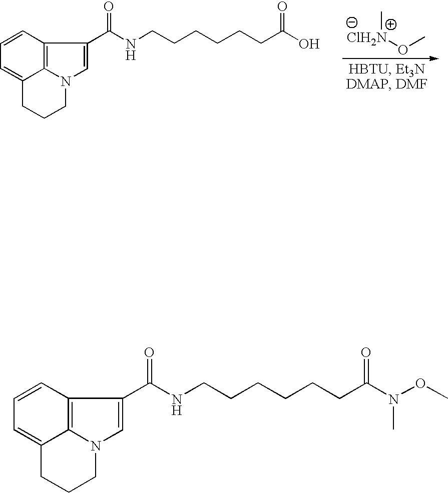

7-[(5,6-Dihydro-4H-pyrrolo[3,2,1-ij]quinoline-1-carbonyl)-amino]-heptanoic acid was reacted with N,O-dimethylhydroxylamine hydrochloride using procedure E. Yield=83% M.p.=61-62° C.; 400 MHz ¹H NMR (CDCl₃) δ: 7.72 (s, 1H), 7.59 (d, J=8.0 Hz, 1H), 7.16 (t, J=8.0 Hz, 1H), 6.98 (d, J=6.8 Hz, 1H), 5.97 (bs, 1H), 4.18 (t, J=6.0 Hz, 2H), 3.65 (s, 3H), 3.52-3.46 (m, 2H), 3.17 (s, 3H), 3.00 (t, J=5.6 Hz, 2H), 2.42 (t, J=5.6 Hz, 2H), 2.27-2.21 (m, 2H), 1.70-1.63 (m, 4H), 1.47-1.41 (m, 4H); LCMS: 372 [M+H].

### Example 7.2

### General Procedure F—Step 2

### Example 7.2.1

### Synthesis of 7-[(5,6-dihydro-4H-pyrrolo[3,2,1-ij]quinolin-1-ylcarbonyl)amino]heptanoic acid

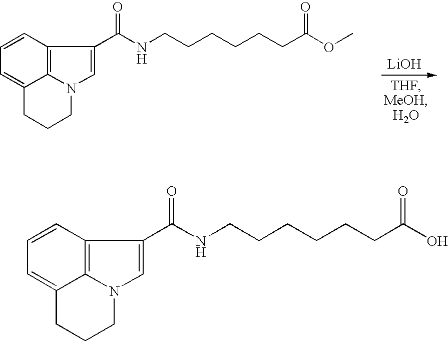

To a solution of methyl 7-[(5,6-dihydro-4H-pyrrolo[3,2,1-ij]quinolin-1-ylcarbonyl)amino]heptanoate (0.70 g, 2.05 mmol) in a mixture of tetrahydrofuran (THF) (3 mL) and methanol (1 mL) was added a solution of lithium hydroxide monohydrate (0.094 g, 2.25 mmol) in water (1 mL). The mixture was stirred at room temperature for 3 hours and then 5 mL of 0.5 N HCl solution was added and diluted with 20 mL of water. Product was extracted with ethyl acetate (200 mL), concentrated and dried under high vacuum at 45° C. overnight to provide 0.70 g (100%) of the title compound as brown solid. It was used without further purification. LCMS: 329 [M+H].

### Example 7.2.2

### Synthesis of 7-[(5,6-dihydro-4H-pyrrolo[3,2,1-ij]quinolin-2-ylcarbonyl)amino]heptanoic acid

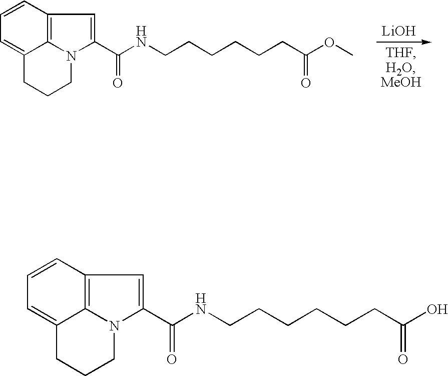

Methyl 7-[(5,6-dihydro-4H-pyrrolo[3,2,1-ij]quinolin-2-ylcarbonyl)amino]heptanoate was hydrolyzed using procedure F to afford 7-[(5,6-dihydro-4H-pyrrolo[3,2,1-ij]quinolin-2-ylcarbonyl)amino]heptanoic acid. LCMS: 329 [M+H].

### Example 7.2.3

### Synthesis of 6-[(5,6-dihydro-4H-pyrrolo[3,2,1-ij]quinolin-1-ylcarbonyl)amino]hexanoic acid

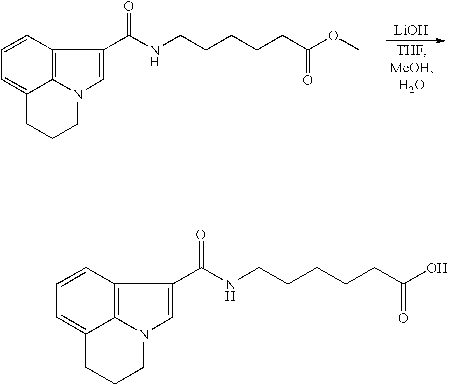

Methyl 6-[(5,6-dihydro-4H-pyrrolo[3,2,1-ij]quinolin-1-ylcarbonyl)amino]hexanoate was hydrolyzed using procedure F to afford 6-[(5,6-dihydro-4H-pyrrolo[3,2,1-ij]quinoline-1-carbonyl)amino]-hexanoic acid as white solid (75%). M.p. 158-161° C. LCMS: 315 [M+H]. 400 MHz ¹H NMR (DMSO-d₆) δ: 11.97 (s, 1H), 7.93 (s, 1H), 7.83 (d, J=8.0 Hz, 1H), 7.75 (t, J=6 Hz, 1H), 7.01 (m, 1H), 6.90 (d, J=6.0 Hz, 1H), 4.19 (t, J=5.8 Hz, 2H), 3.22 (m, 2H), 2.93 (t, J=5.80 Hz, 2H), 2.21 (t, J=7.2 Hz, 2H), 2.13 (t, J=5.8 Hz, 2H), 1.53 (m, 4H), 1.34-1.30 (m, 2H). LCMS: 316[M+H].

### Example 7.2.4

### Synthesis of 7-({[6-(3-methoxyphenyl)imidazo[2,1-b][1,3]thiazol-2-yl]carbonyl}amino)heptanoic acid

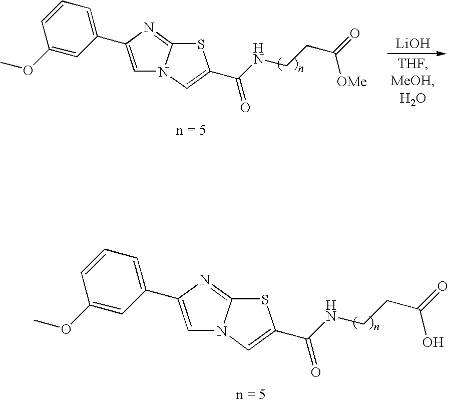

Methyl 7-({[6-(3-methoxyphenyl)imidazo[2,1-b][1,3]thiazol-2-yl]carbonyl}amino)heptanoate was hydrolyzed using procedure F to afford 7-({[6-(3-methoxyphenyl)imidazo[2,1-b][1,3]thiazol-2-yl]carbonyl}amino)heptanoic acid as tan solid. LCMS: 403 [M+H].

### Example 7.2.5

### Synthesis of 7-[benzyl(5,6-dihydro-4H-pyrrolo[3,2,1-ij]quinolin-1-ylcarbonyl)amino]heptanoic acid

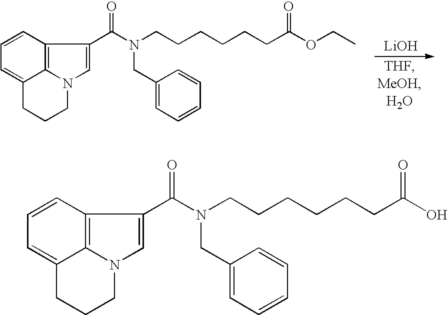

Ethyl 7-[benzyl(5,6-dihydro-4H-pyrrolo[3,2,1-ij]quinolin-1-ylcarbonyl)amino]heptanoate was hydrolyzed following procedure F. Yield=89%, M.p.=45-47° C.; 400 MHz ¹H NMR (CDCl₃) δ: 7.58 (d, J=8.1 Hz, 1H), 7.32 (m, 6H), 7.1 (t, J=7.6 Hz, 1H), 6.94 (d, J=7.0 Hz, 1H), 4.82 (s, 2H), 4.11(t, J=5.7 Hz, 2H), 3.47 (t, J=7.8 Hz, 2H), 2.97 (t, J=6.0 Hz, 2H), 2.25 (m, 4H), 1.59 (m, 4H), 1.28 (m, 4H); LCMC: 419 [M+H].

### Example 7.2.6

### Synthesis of {4-[(5,6-dihydro-4H-pyrrolo[3,2,1-ij]quinolin-1-ylcarbonyl)amino]phenyl}acetic acid

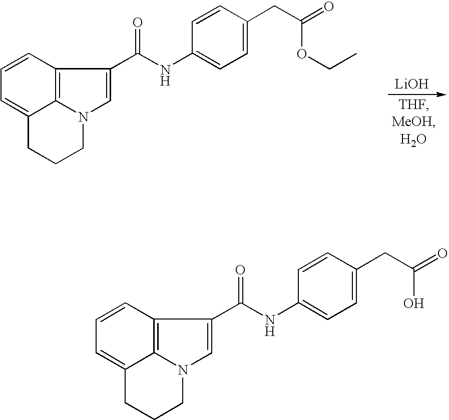

Ethyl {4-[(5,6-dihydro-4H-pyrrolo[3,2,1-ij]quinolin-1-ylcarbonyl)amino]phenyl}acetate was hydrolyzed using procedure F. Yield=74%. 400 MHz ¹H NMR (d6-DMSO) δ: 12.27 (s, 1H), 9.67 (s, 1H), 8.25 (s, 1H), 7.90 (d, J=8 Hz, 1H), 7.69 (d, J=8.4 Hz, 2H), 7.2 (d, J=8.8 Hz, 2H), 7.07 (t, J=7.6 Hz, 1H), 6.95 (d, J=6 Hz, 1H), 4.26 (t, J=5.6 Hz, 2H), 3.52 (s, 2H), 2.96 (t, J=6 Hz, 2H), 2.2-2.14 (m, 2H).

### Example 7.2.7

### Synthesis of 3-[1-(5,6-dihydro-4H-pyrrolo[3,2,1-ij]quinolin-1-ylcarbonyl)piperidin-4-yl]propanoic acid

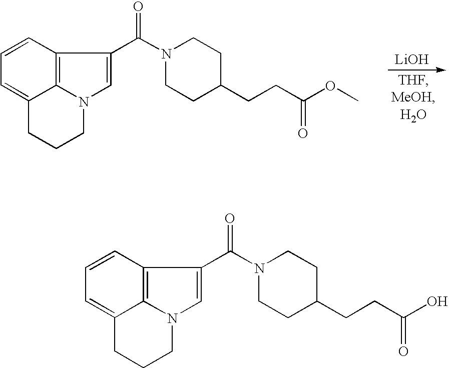

Methyl 3-[1-(5,6-dihydro-4H-pyrrolo[3,2,1-ij]quinolin-1-ylcarbonyl)piperidin-4-yl]propanoate was hydrolyzed using procedure F. Yield=90%. LCMS: 341 [M+H].

### Example 7.2.8

### Synthesis of 7-{[5,6-dihydro-4H-pyrrolo[3,2,1-ij]quinolin-1-yl(oxo)acetyl]amino}heptanoic acid

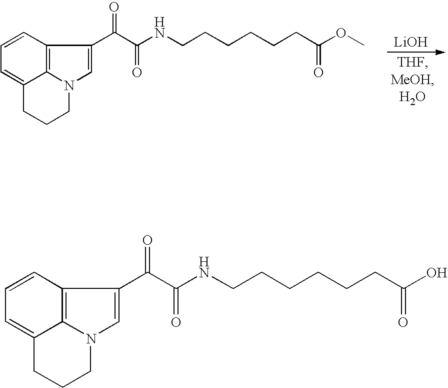

Methyl 7-{[5,6-dihydro-4H-pyrrolo[3,2,1-ij]quinolin-1-yl(oxo)acetyl]amino}heptanoate was hydrolyzed using procedure F. Yield=79%. 400 MHz ¹H NMR (DMSO-d₆) δ: 11.98 (brs, 1H), 8.71 (m, 2H), 8.14 (d, J=7.8 Hz, 1H), 7.22 (t, J=7.8 Hz, 1H), 7.05 (d, J=7.0 Hz, 1H), 4.29 (m, 2H), 3.20 (m, 2H), 2.95 (m, 2H), 2.19 (m, 4H), 1.49 (m, 4H), 1.28 (m, 4H); LCMS: 357 [M+H].

### Example 7.2.9

### Synthesis of 4-{[(5,6-dihydro-4H-pyrrolo[3,2,1-ij]quinolin-1-ylcarbonyl)amino]methyl}benzoic acid

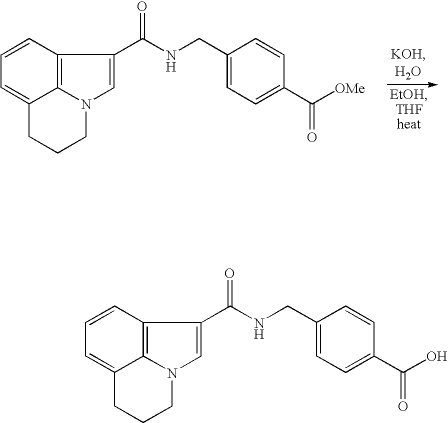

Methyl 4-{[(5,6-dihydro-4H-pyrrolo[3,2,1-ij]quinolin-1-ylcarbonyl)amino]methyl}benzoate (0.616 g, 1.77 mmol) in EtOH (16 ml)/THF (8 ml) was treated with KOH (2.0 g, 35 mmol). The reaction mixture was stirred at 45° C. for 2 hours then poured into water (40 ml). The pH was adjusted to 2 using conc. HCl. The material was extracted with ethyl acetate (2×50 ml). The organic phase was dried over sodium sulfate the concentrated in vacuo, affording a brown solid. The crude material was triturated in Et₂O. The 4-{[(5,6-dihydro-4H-pyrrolo[3,2,1-ij]quinolin-1-ylcarbonyl)amino]methyl}benzoic acid was obtained as a pale yellow solid (0.507 g, 86%). M.p.=255-256° C. 400 MHz ¹H NMR (DMSO-d₆) δ: 8.46 (t, J=5.9 Hz, 1H), 8.03 (s, 1H), 7.90 (d, J=8.1 Hz, 2H), 7.86 (d, J=8.1 Hz, 1H), 7.44 (d, J=8.4 Hz, 2H), 7.04 (t, J=7.5 Hz, 1H), 6.92 (d, J=7.0 Hz, 1H), 4.54 (d, J=6.2 Hz, 2H), 4.21 (t, J=5.5 Hz, 2H), 2.94 (t, J=5.9 Hz, 2H), 2.14 (quintuplet, J=5.9 Hz, 2H). LCMS: 335 [M+H].

### Example 7.3

### General Procedure G—Step 3

### Example 7.3.1

### Synthesis of 7-{[5,6-dihydro-4H-pyrrolo[3,2,1-ij]quinolin-1-yl(oxo)acetyl]amino}-N-(tetrahydro-2H-pyran-2-yloxy)heptanamide

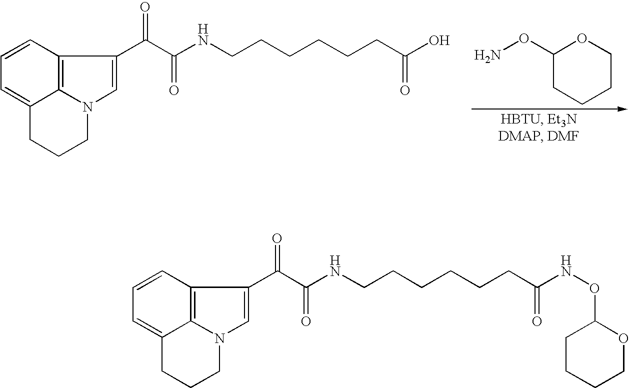

To a solution of 7-{[5,6-dihydro-4H-pyrrolo[3,2,1-ij]quinolin-1-yl(oxo)acetyl]amino}heptanoic acid (773 mg, 2.17 mmol) in anhydrous DMF (30 mL) was added triethylamine (0.6 mL, 4.34 mmol), O-(tetrahydro-2H-pyran-2-yl)hydroxylamine (305 mg, 2.6 mmol), and HBTU (986 mg, 2.6 mmol). The reaction mixture was allowed to stir for at room temperature for 18 h. The reaction mixture was diluted with dichloromethane (200 mL) and washed with saturated sodium bicarbonate solution (100 mL). The aqueous layer was extracted with DCM (3×100 mL), washed with brine (100 mL), dried over sodium sulfate, and evaporated under reduced pressure. The crude product was purified by flash column chromatography (SiO₂, 1% EtOAc in hexanes to 40% EtOAc in hexanes) to afford 80% (1.04 g) of as a yellow solid. M.p.=140-142° C.; 400 MHz ¹H NMR (CDCl₃) δ: 8.95 (s, 1H), 8.63 (brs, 1H), 8.14 (d, J=8.2 Hz, 1H), 7.59 (brs, 1H), 7.22 (t, J=8.2 Hz, 1H), 7.07 (d, J=7.0 Hz, 1H), 5.30 (brs, 1H), 4.26 (t, J=5.8 Hz, 2H), 3.96 (brs, 1H), 3.62 (m, 1H), 3.39 (m, 2H), 3.04 (t, J=5.8 Hz, 2H), 2.29 (t, J=5.8 Hz, 2H), 2.26 (brs, 2H), 1.81 (m, 3H), 1.68 (m, 7H), 1.39 (m, 4H); LCMS: 456 [M+H].

### Example 7.3.2

### Synthesis of 5,6-Dihydro-4H-pyrrolo3,2,1-ij]quinoline-2-carboxylic acid[6-(tetrahydro-pyran-2-yloxycarbamoyl)-hexyl]-amide

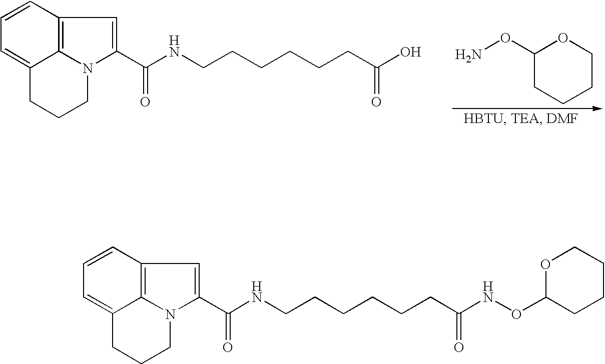

7-[(5,6-dihydro-4H-pyrrolo[3,2,1-ij]quinolin-2-ylcarbonyl)amino]heptanoic acid was reacted with O-(tetrahydro-2H-pyran-2-yl)hydroxylamine using procedure G. LCMS: 429 [M+H].

### Example 7.3.3

### Synthesis of N-(4-{2-oxo-2-[(tetrahydro-2H-pyran-2-yloxy)amino]ethyl}phenyl)-5,6-dihydro-4H-pyrrolo[3,2,1-ij]quinoline-1-carboxamide

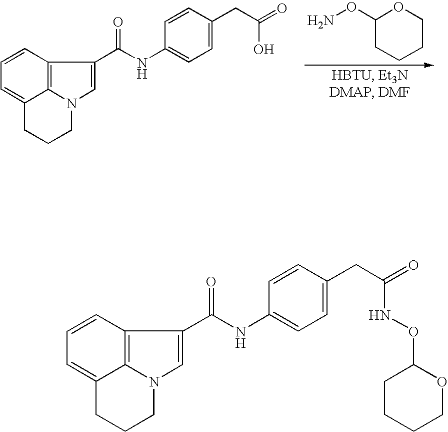

{4-[(5,6-Dihydro-4H-pyrrolo[3,2,1-ij]quinoline-1-carbonyl)-amino]-phenyl}-acetic acid (275 mg, 0.82 mmol) was reacted with O-(tetrahydro-2H-pyran-2-yl)hydroxylamine using procedure G. Yield=85%. 400 MHz ¹H NMR (d6-DMSO) δ: 11.22 (s, 1H), 9.66 (s, 1H), 8.25 (s, 1H), 7.90 (d, J=8.4 Hz, 1H), 7.68 (d, J=8.8 Hz, 2H), 7.2 (d, J=8.8 Hz, 2H), 7.07 (t, J=7.2 Hz, 1H), 6.95 (d, J=6.8 Hz, 1H), 4.83 (s, 1H), 4.25 (t, J=5.6 Hz, 2H), 3.97-3.91 (m, 1H), 3.56-3.51 (m, 1H), 3.29 (s, 2H), 2.96 (t, J=6 Hz, 2H), 2.2-2.14 (m, 2H), 1.7-1.46 (m, 6H); LCMS: 434 [M+H].

### Example 7.3.4

### Synthesis of 6-(3-methoxyphenyl)-N-{7-oxo-7-[(tetrahydro-2H-pyran-2-yloxy)amino]heptyl}imidazo[2,1-b][1,3]thiazole-2-carboxamide

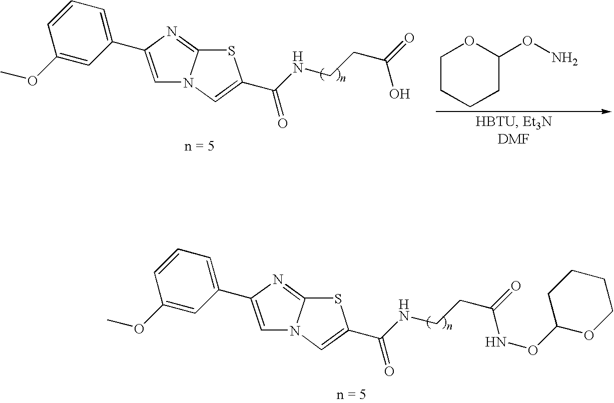

7-({[6-(3-methoxyphenyl)imidazo[2,1-b][1,3]thiazol-2-yl]carbonyl}amino)heptanoic acid was reacted with O-(tetrahydro-2H-pyran-2-yl)hydroxylamine using procedure G. LCMS: 501 [M+H].

### Example 7.3.5

### Synthesis of N-{4-[(benzyloxy)carbamoyl]benzyl}-5,6-dihydro-4H-pyrrolo[3,2,1-ij]quinoline-1-carboxamide

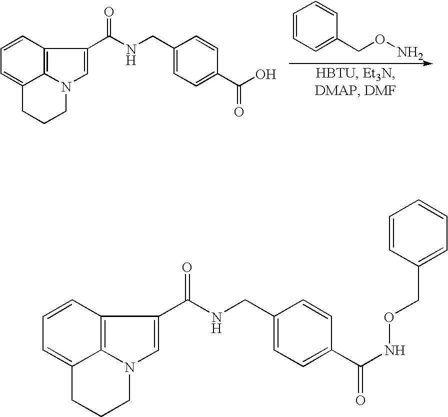

4-{[(5,6-dihydro-4H-pyrrolo[3,2,1-ij]quinolin-1-ylcarbonyl)amino]methyl}benzoic acid was reacted with O-benzylhydroxylamine using procedure G. N-(4-{[(benzyloxy)amino]carbonyl}benzyl)-5,6-dihydro-4H-pyrrolo[3,2,1-ij]quinoline-1-carboxamide was obtained as a pale yellow solid. Yield=92%. M.p.=234-235° C. 400 MHz ¹H NMR (DMSO-d₆) δ: 11.73 (s, 1H), 8.44 (t, J=6.0 Hz, 1H), 8.02 (s, 1H), 7.85 (d, J=8.1 Hz, 1H), 7.70 (d, J=8.1 Hz, 2H), 7.50-7.30 (m, 7H), 7.03 (t, J=7.5 Hz, 1H), 6.92 (d, J=7.0 Hz, 1H), 4.91 (s, 2H), 4.51 (d, J=5.9 Hz, 2H), 4.21 (t, J=5.7 Hz, 2H), 2.94 (t, J=5.9 Hz, 2H), 2.14 (quintuplet, J=5.3 Hz, 2H). LCMS: 440 [M+H].

### Example 7.4

### General Procedure H—Step 4

### Example 7.4.1

### Synthesis of N-{4-[(hydroxyamino)carbonyl]benzyl}-5,6-dihydro-4H-pyrrolo[3,2,1-ij]quinoline-1-carboxamide

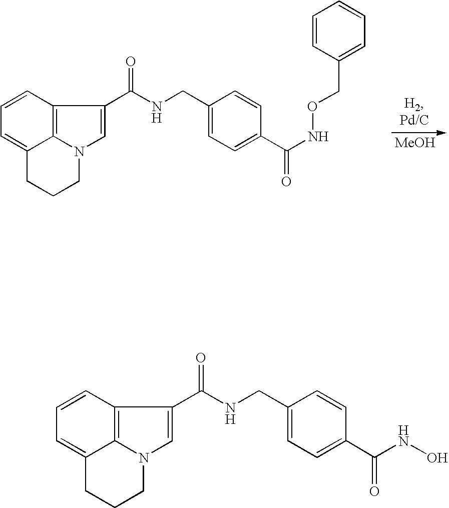

The N-(4-{[(benzyloxy)amino]carbonyl}benzyl)-5,6-dihydro-4H-pyrrolo[3,2,1-ij]quinoline-1-carboxamide (0.570 g, 1.30 mmol) in MeOH (40 ml), containing Pd/C 10% (0.4 g wet material, 0.13 mmol), was subjected to H₂ at atmospheric pressure for 4 hours. The catalyst was filtered off. The mixture was concentrated in vacuo, affording a brown solid. The crude material was triturated in ethyl acetate. The N-{4-[(hydroxyamino)carbonyl]benzyl}-5,6-dihydro-4H-pyrrolo[3,2,1-ij]quinoline-1-carboxamide was obtained as a beige solid (0.372 g, 82%). M.p.=191-192° C. 400 MHz ¹H NMR (DMSO-d₆) δ: 11.17 (s, 1H), 8.99 (s, 1H), 8.44 (t, J=6.0 Hz, 1H), 8.02 (s, 1H), 7.86 (d, J=8.1 Hz, 1H), 7.70 (d, J=8.1 Hz, 2H), 7.39 (d, J=8.1 Hz, 2H), 7.03 (t, J=7.7 Hz, 1H), 6.92 (d, J=7.0 Hz, 1H), 4.50 (d, J=5.9 Hz, 2H), 4.21 (t, J=5.5 Hz, 2H), 2.94 (t, J=5.9 Hz, 2H), 2.14 (quintuplet, J=5.3 Hz, 2H). LCMS: 350 [M+H].

### Example 7.4.2

### Synthesis of N-[5-(hydroxyamino)-5-oxopentyl]-5,6-dihydro-4H-pyrrolo[3,2,1-ij]quinoline-1-carboxamide

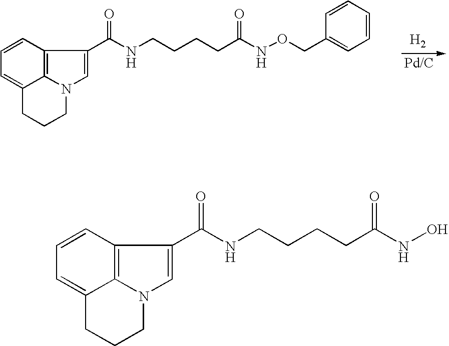

N-{5-[(benzyloxy)amino]-5-oxopentyl}-5,6-dihydro-4H-pyrrolo[3,2,1-ij]quinoline-1-carboxamide was subjected to conditions from procedure H to yield 5,6-Dihydro-4H-pyrrolo[3,2,1-ij]quinoline-1-carboxylic acid (4-hydroxycarbamoyl-butyl)-amide. M.p. 179-181° C., 400 MHz ¹H NMR (DMSO-d₆) δ: 10.35 (s, 1H), 8.67 (d, J=1.60 Hz, 1H), 7.94 (s, 1H), 7.83 (d, J=7.83 Hz, 1H), 7.79 (t, J=5.6 Hz, 1H), (dd, J=7.2, 0.8 Hz, 1H), 6.90 (d, J=6.80 Hz, 1H), 4.19 (t, J=5.60 Hz, 2H), 3.23 (m, 2H), 2.93 (t, J=6.0 Hz, 2H), 2.13 (t, J=5.6 Hz, 2H), 1.98 (t, J=7.20 Hz, 2H), 1.54-1.48 (m, 4H). LCMS: 316 [M+H].

### Example 7.4.3

### Synthesis of N-[8-(hydroxyamino)-8-oxooctyl]-5,6-dihydro-4H-pyrrolo[3,2,1-ij]quinoline-1-carboxamide

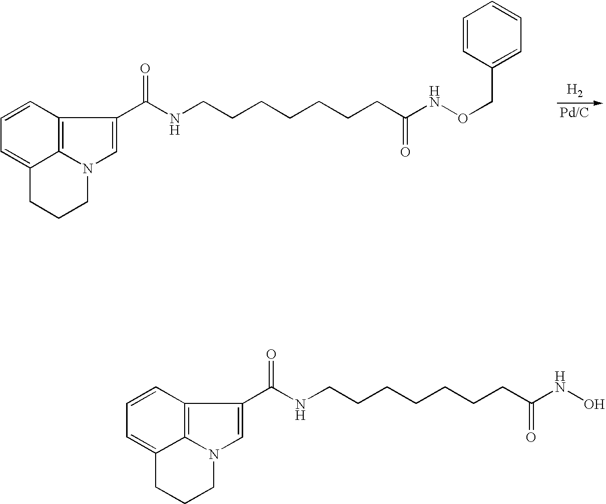

N-{8-[(benzyloxy)amino]octyl}-5,6-dihydro-4H-pyrrolo[3,2,1-ij]quinoline-1-carboxamide was subjected to hydrogenation conditions from procedure H to yield 5,6-Dihydro-4H-pyrrolo[3,2,1-ij]quinoline-1-carboxylic acid (7-hydroxycarbamoyl-heptyl)-amide. Yield=42%. M.p.=174-176° C. 400 MHz ¹H NMR (DMSO-d,₆) δ: 10.3 (s, 1H), 8.6 (d, J=1.60 Hz 1H), 7.94 (s, 1H), 7.83 (d, J=7.60 Hz, 1H), 7.76 (m, 1H), 7.00 (dd, J=7.6, 6.8 Hz, 1H), 6.90 (d, J=6.80 Hz, 1H), 4.19 (t, J=5.60 Hz, 2H), 3.23 (m, 2H), 2.93 (t, J=5.80 Hz, 2H), 2.13 (m, 2H), 1.93 (t, J=7.20 Hz, 2H), 1.49-1.46 (m, 4H), 1.29 (m, 6H). LCMS: 358 [M+H].

### Example 7.4.4

### Synthesis of 4-{[5,6-dihydro-4H-pyrrolo[3,2,1-ij]quinolin-1-yl(oxo)acetyl]amino}-N-hydroxybutanamide

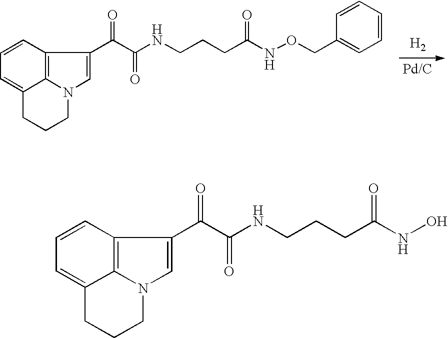

N-(benzyloxy)-4-{[5,6-dihydro-4H-pyrrolo[3,2,1-ij]quinolin-1-yl(oxo)acetyl]amino}butanamide was hydrogenated using procedure H. Yield=78%. M.p.=95-97° C.; 300 MHz ¹H NMR (DMSO-d,₆) δ: 10.39 (s, 1H), 8.73 (bs, 3H), 7.95 (d, J=7.5 Hz, 1H), 7.18 (t, J=7.2 Hz, 1H), 7.05 (d, J=6.9 , 1H), 4.29 (t, J=5.1 Hz, 2H), 3.23-3.17 (m, 2H), 2.96 (t, J=5.4 Hz, 2H), 2.16-2.12 (m, 2H), 2.00 (t, J=6.9 Hz, 2H), 1.77-1.71 (m, 2H); LCMS: 330 [M+H].

### Example 7.4.5

### Synthesis of 4-{[5,6-dihydro-4H-pyrrolo[3,2,1-ij]quinolin-1-yl(oxo)acetyl]amino}-N-hydroxypentanamide

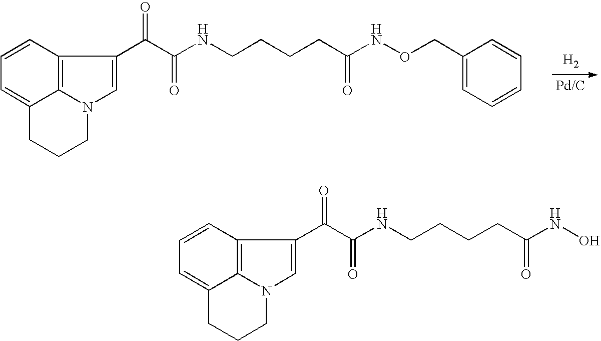

N-(benzyloxy)-4-{[5,6-dihydro-4H-pyrrolo[3,2,1-ij]quinolin-1-yl(oxo)acetyl]amino}pentanamide was hydrogenated under the conditions for procedure H. Yield=80%. M.p.=135-137° C.; 300 MHz ¹H NMR (DMSO-d₆) δ: 10.35 (s, 1H), 8.72 (bs, 2H), 8.68 (s, 1H), 7.95 (d, J=8.1 Hz, 1H), 7.18 (t, J=7.5 Hz, 1H), 7.05 (d, J=6.9, 1H), 4.32-4.27 (m, 2H), 3.23-3.17 (m, 2H), 2.96 (t, J=5.7 Hz, 2H), 2.16-2.12 (m, 2H), 1.96 (t, J=6.3 Hz, 2H), 1.50-1.48 (m, 2H). LCMS: 344 [M+H].

### Example 7.4.6

### Synthesis of 6-{[5,6-dihydro-4H-pyrrolo[3,2,1-ij]quinolin-1-yl(oxo)acetyl]amino}-N-hydroxyhexanamide

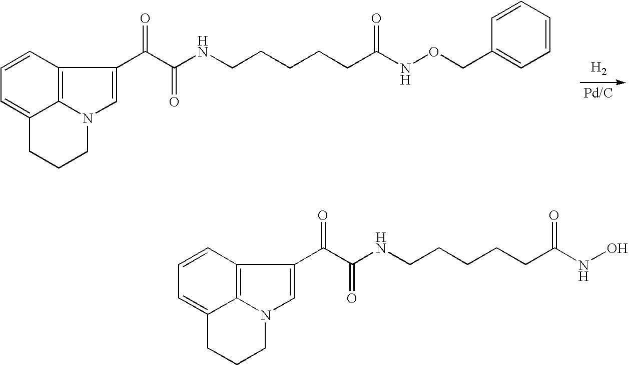

6-[2-Oxo-2-(2a,3,4,5-tetrahydro-acenaphthylen-1-yl)-acetylamino]-hexanoic acid benzyloxy—was subjected to hydrogenation conditions using procedure H. M.p.=150-155° C.; 400 MHz ¹H NMR (DMSO-d₆) δ: 10.33 (s, 1H), 8.72-8.66 (m, 3H), 7.94 (d, J=8.0 Hz, 1H), 7.18 (t, J=7.6 Hz, 1H), 7.04 (d, J=7.2 Hz, 1H), 4.29 (t, J=5.6 Hz, 2H), 3.09 (d, J=7.2 Hz, 2H), 2.95 (t, J=6.0 Hz, 2H), 2.17-2.11 (m, 2H), 1.94 (t, J=7.2H, 2H), 1.54-1.46 (m, 4H), 1.25-1.23 (m, 2H); LCMS: 358 [M+H].

### Example 7.5

### General Procedure I—Step 4

### Example 7.5.1

### Synthesis of 7-{[5,6-dihydro-4H-pyrrolo[3,2,1-ij]quinolin-1-yl(oxo)acetyl]amino}-N-hydroxyheptanamide

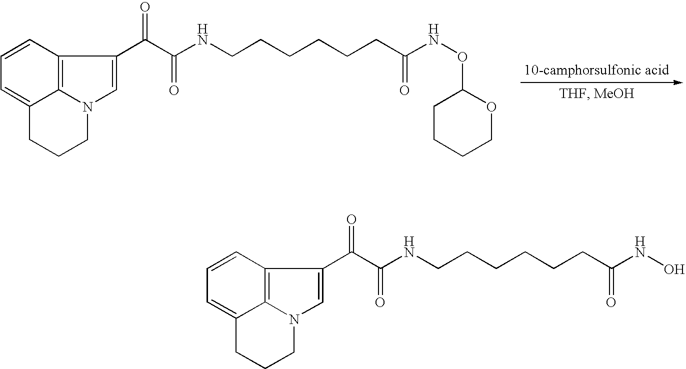

7-[2-(5,6-Dihydro-4H-pyrrolo[3,2,1-ij]quinolin-1-yl)-2-oxo-acetylamino]-heptanoic acid (tetrahydro-pyran-2-yloxy)-amide (740 mg, 1.62 mmol) was dissolved in THF:methanol mixture (3:1 v/v) (250 mL) and camphorsulfonic acid (415 mg, 1.78 mmol) was added. The reaction mixture was allowed to stir for 2 hour at room temperature. The reaction mixture was diluted with water (100 mL), DCM (200 mL) and the layers were separated. The aqueous layer was extracted with DCM (4×100 mL), washed with brine (100 mL), dried over sodium sulfate, and evaporated to dryness. The crude product was dissolved in 5% MeOH in DCM and purified by flash column chromatography (SiO₂, 0% MeOH in DCM to 8% % MeOH in DCM) to afford 49% (301 mg) of as a white solid. M.p.=158-159° C.; 400 MHz ¹H NMR (DMSO-d₆) δ: 10.19 (brs, 1H), 8.69 (s, 1H), 8.49 (m, 2H), 7.95 (d, J=8.2 Hz, 1H), 7.18 (t, J=8.2 Hz, 1H), 7.04 (d, J=8.2 Hz, 1H); 4.29 (t, J=5.8 Hz, 2H), 3.20 (m, 2H), 2.95 (t, J=8.2 Hz, 2H), 2.15 (m, 2H), 1.95 (m, 2H), 1.52 (m, 4H), 1.30 (m, 4h); LCMS: 372 [M+H].

### Example 7.5.2

### Synthesis of N-[7-(hydroxyamino)-7-oxoheptyl]-5,6-dihydro-4H-pyrrolo[3,2,1-ij]quinoline-2-carboxamide

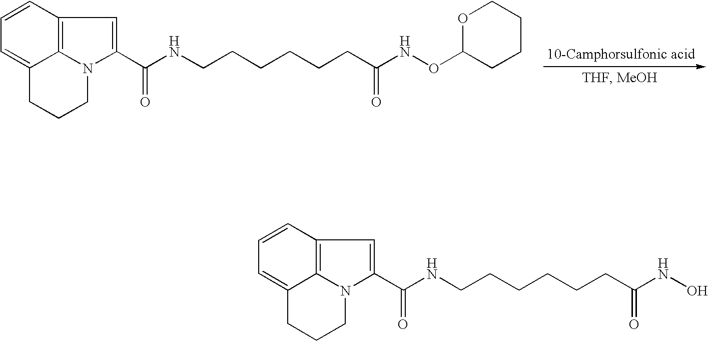

5,6-Dihydro-4H-pyrrolo[3,2,1-ij]quinoline-2-carboxylic acid[6-(tetrahydro-pyran-2-yloxycarbamoyl)-hexyl]-amide was reacted with 10-camphorsulfonic acid using procedure I. M.p.=160-161° C.; 400 MHz ¹H NMR (DMSO-d₆) δ: 10.33 (s, 1H), 8.66 (s, 1H), 8.42-8.39 (t, J=5.86 Hz, 1H), 7.42-7.39 (d, J=7.44 Hz, 1H), 7.02 (s, 1H), 6.99-6.92 (m, 2H), 4.47 (t, J=5.86 Hz, 2H), 3.25-3.2 (m, 2H), 2.92 (t, J=5.86 Hz, 2H), 2.12-2.07 (m, 2H), 1.94 (t, J=7.43 Hz, 2H), 1.53-1.25 (m, 8H); LCMS: 344 [M+H].

### Example 7.5.3

### Synthesis of N-[7-(hydroxyamino)-7-oxoheptyl]-6-(3-methoxyphenyl)imidazo[2,1-b][1,3]thiazole-2-carboxamide

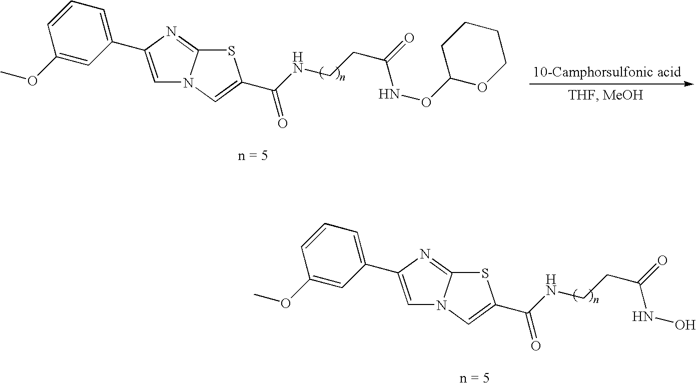

N-[7-(hydroxyamino)-7-oxoheptyl]-6-(3-methoxyphenyl)imidazo[2,1-b][1,3]thiazole-2-carboxamide was synthesized using 6-(3-methoxyphenyl)-N-{7-oxo-7-[(tetrahydro-2H-pyran-2-yloxy)amino]heptyl}imidazo[2,1-b][1,3]thiazole-2-carboxamide following procedure I. M.p.=209-211° C.; 400 MHz ¹H NMR (DMSO-d₆) δ: 10.31 (brs, 1H), 8.64-8.68 (m, 2H), 8.55 (s, 1H), 8.36 (s, 1H), 7.39-7.41 (m, 2H), 7.27-7.31 (m, 1H), 6.81-6.84 (m, 1H); 3.78 (s, 3H), 3.19-3.26 (m, 2H), 1.90-1.95 (t, J=7.43 Hz, 2H), 1.24-1.52 (m, 8H); LCMS: 417 [M+H].

### Example 7.6

### General Procedure J—Step 4

### Synthesis of N-{4-[2-(hydroxyamino)-2-oxoethyl]phenyl}-5,6-dihydro-4H-pyrrolo[3,2,1-ij]quinoline-1-carboxamide

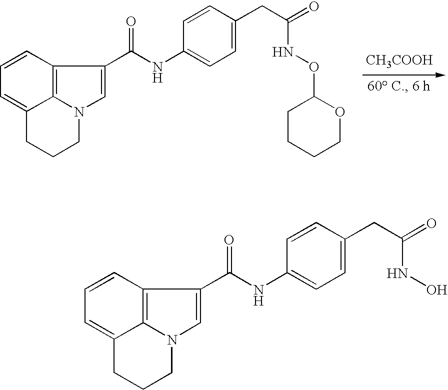

A solution of N-(4-{2-oxo-2-[(tetrahydro-2H-pyran-2-yloxy)amino]ethyl}phenyl)-5,6-dihydro-4H-pyrrolo[3,2,1-ij]quinoline-1-carboxamide (300 mg, 0.69 mmol) in tetrahydrofuran (5 ml) acetic acid (10 ml) and water (3 ml) was heated to 60° C. for 6 hours. The mixture was evaporated to dryness and the resulting pale brown solid recrystalized from methanol/water to give as an off white solid (114 mg, 47%). M.p.=199-202° C., 400 MHz ¹H NMR (d6-DMSO) δ: 10.64 (s, 1H), 9.66 (s, 1H), 8.82 (s, 1H), 8.25 (s, 1H), 7.90 (d, J=7.6 Hz, 1H), 7.68 (d, J=8.8 Hz, 2H), 7.2 (d, J=8.8 Hz, 2H), 7.07 (t, J=7.2 Hz, 1H), 6.95 (d, J=6.4 Hz, 1H), 4.25 (t, J=5.2 Hz, 2H), 3.24 (s, 2H), 2.96 (t, J=6 Hz, 2H), 2.2-2.14 (m, 2H); LCMS: 350 [M+H].

### Example 7.7

### General Procedure K

### Synthesis of 3-[1-(5,6-dihydro-4H-pyrrolo[3,2,1-ij]quinolin-1-ylcarbonyl)piperidin-4-yl]-N-hydroxypropanamide

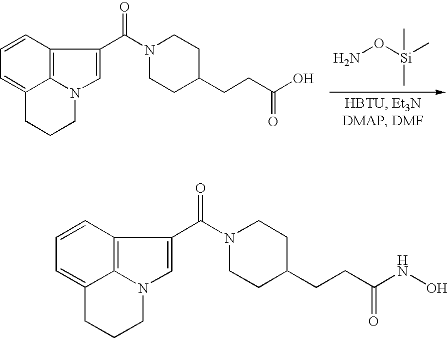

3-[1-(5,6-dihydro-4H-pyrrolo[3,2,1-ij]quinolin-1-ylcarbonyl)piperidin-4-yl]propanoic acid was reacted with O-(trimethylsilyl)hydroxylamine using procedure G to afford the desired product 3-[1-(5,6-Dihydro-4H-pyrrolo[3,2,1-ij]quinoline-1-carbonyl)-piperidin-4-yl]-N-hydroxy-propionamide. M.p.=110-113° C.; 400 MHz ¹H NMR (DMSO_{d6}) δ: 10.34 (s, 1H), 8.66 (s, 1H), 7.64 (s, 1H), 7.38 (d, J=10.8 Hz, 1H), 6.99 (t, J=9.6 Hz, 1H), 6.88 (d, J=10.8 Hz, 1H), 4.24 (d, J=12.4 Hz, 2H), 4.17 (t, t, J=5.6 Hz, 2H), 2.95-2.66 (m, 4H), 2.11 (pent, J=5.6 Hz, 2H), 1.96 (t, J=6.0 Hz, 3H), 1.66 (d, J=11.6 Hz, 2H), 1.44 (t, J=6.0 Hz, 3H), 1.26-0.94 (m, 2H); LCMS: 356 [M+H].

### Example 7.8

### General Procedure L

### Example 7.8.1

### Synthesis of N-[6-(hydroxyamino)-6-oxohexyl]-5,6-dihydro-4H-pyrrolo[3,2,1-ij]quinoline-1-carboxamide

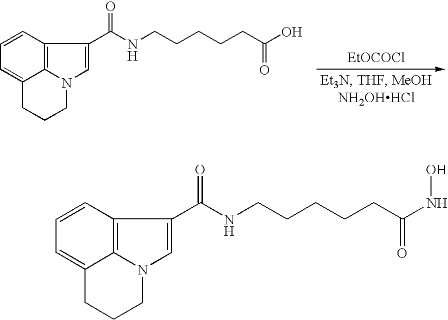

6-[(5,6-dihydro-4H-pyrrolo[3,2,1-ij]quinolin-1-ylcarbonyl)amino]hexanoic acid (0.9 g, 2.86 mmol) was suspended in anhydrous THF (20 mL) and allowed to stir under nitrogen for 1 min whereupon, ethylchloroformate (0.33 ml, 4.28 mmol) and triethylamine (0.6 ml, 4.29 mmol) were added and the cloudy mixture was stirred for 5 min. A mixture of hydroxylamine hydrochloride (0.394 g, 5.72 mmol) in triethylamine (0.6 ml, 4.29 mmol) in methanol (4 ml) was added and allowed to stir under nitrogen for overnight. Upon completion of reaction THF (40 ml) was added and centrifuged. The solvent was decanted and the process was repeated twice. The white solid obtained was sonnicated with dilute HCl and the HCl layer was decanted. The process was repeated twice to remove unreacted hydroxylamine to afford (5,6-dihydro-4H-pyrrolo[3,2,1-ij]quinoline-1-carboxylic acid (5-hydroxy carbamoyl-pentyl)-amide as white solid (0.50 g, 50%). 400 MHz ¹H NMR (DMSO-d₆) δ: 10.3 (s, 1H), 8.6 (s, 1H), 7.93 (s, 1H), 7.83 (d, J=7.82 Hz, 1H), 7.59 (t, J=6 Hz, 1H), 7.01 (t, J=7.6 Hz, 1H), 6.89 (d, J=6.65 Hz, 1H), 4.18 (t, J=5.47 Hz, 2H), 3.22 (q, J=12.91, 7.04 Hz, 2H), 2.92 (t, J=5.86 Hz, 2H), 2.13 (m, 2H), 1.95 (t, J=7.43 Hz, 2H), 1.55-1.46 (m, 4H), 1.32-1.24 (m, 2H). LCMS: 331[M+H].

### Example 7.8.2

### Synthesis of N-[7-(hydroxyamino)-7-oxoheptyl]-5,6-dihydro-4H-pyrrolo[3,2,1-ij]quinoline-1-carboxamide

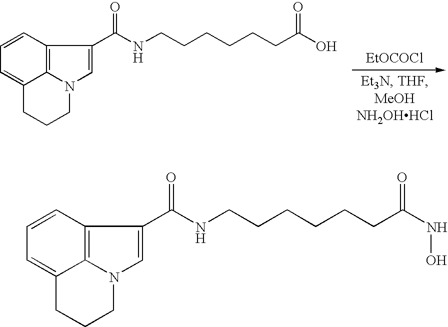

7-[(5,6-Dihydro-4H-pyrrolo[3,2,1-ij]quinoline-1-carbonyl)-amino]-heptanoic acid was reacted with hydroxylamine hydrochloride using procedure L. Yield=40%. M.p.=205-206° C.; 400 MHz ¹H NMR (DMSO-d₆) δ: 10.33 (s, 1H), 8.66 (s, 1H), 7.94 (s, 1H), 7.83 (d, J=8.0 Hz, 1H), 7.75 (t, J=5.6 Hz, 1H), 7.01 (t, J=7.2 Hz, 1H), 6.89 (d, J=6.8 Hz, 1H), 4.18 (t, J=5.2 Hz, 2H), 3.24-3.2 (m, 2H), 2.92 (t, J=5.6 Hz, 2H), 2.12 (t, J=5.6 Hz, 2H), 1.94 (t, J=7.6 Hz, 2H), 1.51-1.48 (m, 4H), 1.28 (bs, 4H); LCMS: 344 [M+H].

### Example 7.8.3

### Synthesis of N-benzyl-N-[7-(hydroxyamino)-7-oxoheptyl]-5,6-dihydro-4H-pyrrolo[3,2,1-ij]quinoline-1-carboxamide

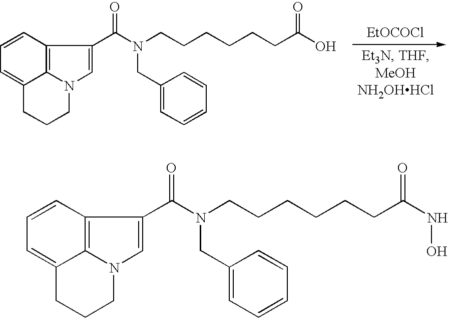

7-[Benzyl-(5,6-dihydro-4H-pyrrolo[3,2,1-ij]quinoline-1-carbonyl)-amino]-heptanoic acid was reacted with hydroxylamine hydrochloride using procedure L. Yield=32%. M.p.=71-73° C.; 400 MHz ¹H NMR (DMSO-d₆) 10.29 (brs, 1H), 8.62 (brs, 1H), 7.59 (s, 1H), 7.48 (d, J=8.0 Hz, 1H), 7.32 (d, J=7.3 Hz, 1H), 7.25 (d, J=7.3 Hz, 3H), 6.99 (t, J=7.7 Hz, 1H), 6.89 (d, J=7.3 Hz, 1H), 4.73 (s, 2H), 4.15 (t, J=5.8 Hz, 2H), 2.92 (t, J=5.8 Hz, 2H), 2.10 (m, 2H), 1.85 (t, J=7.7 Hz, 2H), 1.52 (m, 3H), 1.39 (m, 3H), 1.15 (m, 6H). LCMS: 434 [M+H].

### Example 7.8.4

### Synthesis of N-{7-[(2-aminophenyl)amino]-7-oxoheptyl}-5,6-dihydro-4H-pyrrolo[3,2,1-ij]quinoline-1-carboxamide

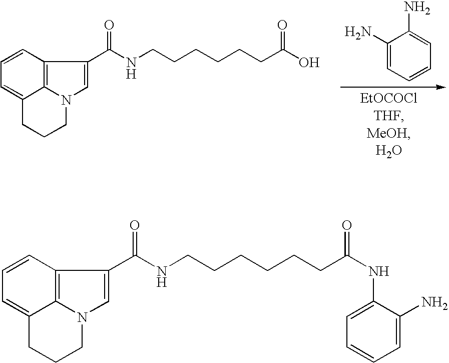

To a solution of 7-[(5,6-Dihydro-4H-pyrrolo[3,2,1-ij]quinoline-1-carbonyl)-amino]-heptanoic acid was reacted with benzene-1,2-diamine using procedure L. Yield=53%. M.p.=144-145° C.; 400 MHz ¹H NMR (CDCl₃) δ: 7.69 (s, 1H), 7.68 (s, 1H), 7.62 (d, J=8.4 Hz, 1H), 7.21 (d, J=7.6 Hz, 1H), 7.16 (t, J=7.2 Hz, 1H), 7.05-7.02 (m 1H), 6.98 (d, J=7.6 Hz, 1H), 6.78-6.75 (m, 2H), 6.04 (t, J=5.2 Hz, 1H), 4.14 (t, J=5.2 Hz, 2H), 3.53-3.49 (m, 2H), 3.00 (t, J=6.0 Hz, 2H), 2.40 (t, J=7.6 Hz, 2H), 2.25-2.21 (m, 2H), 1.77 (t, J=8.0 Hz, 2H), 1.68-1.64 (m, 4H), 1.48-1.45 (m, 4H). LCMS: 419 [M+H].

### Example 7.8.5

### Synthesis of N-{7-[(2-amino-4 5-dichlorophenyl)amino]-7-oxoheptyl}-5,6-dihydro-4H-pyrrolo[3,2,1-ij]quinoline-1-carboxamide

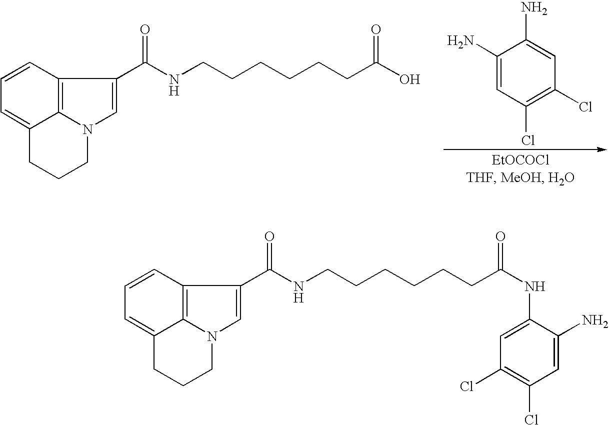

To a solution of 7-[(5,6-Dihydro-4H-pyrrolo[3,2,1-ij]quinoline-1-carbonyl)-amino]-heptanoic acid was reacted with 4,5-dichloro-benzene-1,2-diamine using procedure L. Yield=34%. M.p.=124-125° C.; 400 MHz ¹H NMR (DMSO-d₆) δ: 9.11 (s, 1H), 7.93 (s, 1H), 7.82 (d, J=8.0 Hz, 1H), 7.77 (d, J=5.2 Hz, 1H), 7.54 (s, 1H), 7.01 (t, J=8.0 Hz, 1H), 6.90-6.89 (m 2H), 5.33 (s, 2H), 4.18 (t, J=5.6 Hz, 2H), 3.26-3.21 (m, 2H), 2.92 (t, J=6.0 Hz, 2H), 2.32 (t, J=7.6 Hz, 2H), 2.14-2.11 (m, 2H), 1.59 (bs, 2H), 1.52 (bs, 2H), 1.34(bs, 4H). LCMS: 487 [M+H].

### Example 8

### General Scheme 4

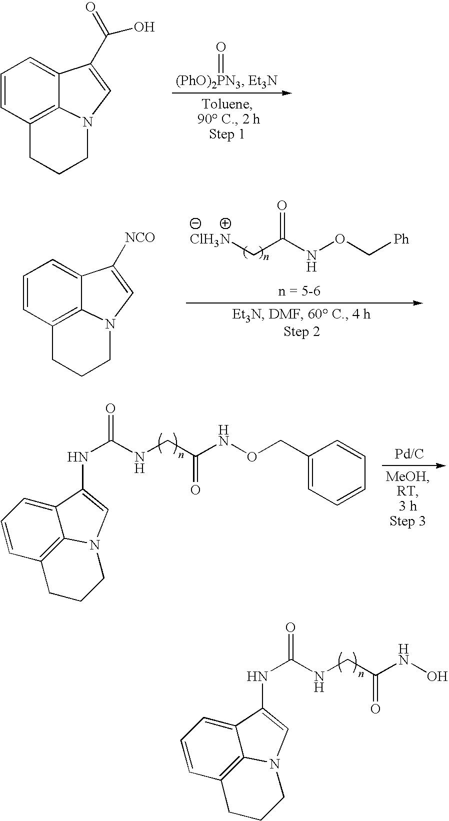

### Example 8.1

### Step 1

### Synthesis of 1-isocyanato-5,6-dihydro-4H-pyrrolo[3,2,1-ij]quinoline

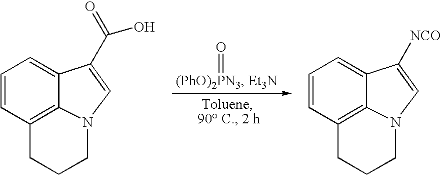

To a solution of 5,6-Dihydro-4-H-pyrrolo[3,2,1-ij]quinoline-1-carboxylic acid (1.0 g, 5.0 mmol) and triethylamine (1.07 ml, 5.0 mmol) in anhydrous toluene was added diphenylphosphonic azide (1.44 g, 5.25 mmol). The reaction was heated at 90° C. for 2 h. The solvent was removed under reduced pressure. The crude product was ready for the next step.

### Example 8.2

### General Procedure M—Step 2

### Example 8.2.1

### Synthesis of N-(benzyloxy)-6-[5,6-dihydro-4H-pyrrolo[3,2,1-ij]quinolin-1-ylcarbamoyl)amino]hexanamide

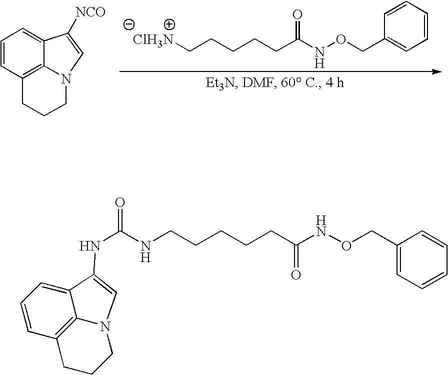

To a solution of the 1-isocyanato-5,6-dihydro-4H-pyrrolo[3,2,1-ij]quinoline (0.139 mg, 0.70 mmol) and triethylamine (0.192 ml, 1.38 mmol) in anhydrous DMF (6 ml) was added the N-(benzyloxy)-6-(chloroamino)hexanamide (0.263 g, 0.966 mmol). The reaction was stirred at 60° C. for 4 hours. The solvent was removed under reduced pressure. The crude product was purified by flash column chromatography (SiO₂, 5% DCM in methanol) to give the desired product as a pale yellow solid (0.20 g, 67%). LCMS: 435 [M+H].

### Example 8.2.2

### Synthesis of N-(benzyloxy)-7-[(5,6-dihydro-4H-pyrrolo[3,2,1-ij]quinolin-1-ylcarbamoyl)amino]heptanamide

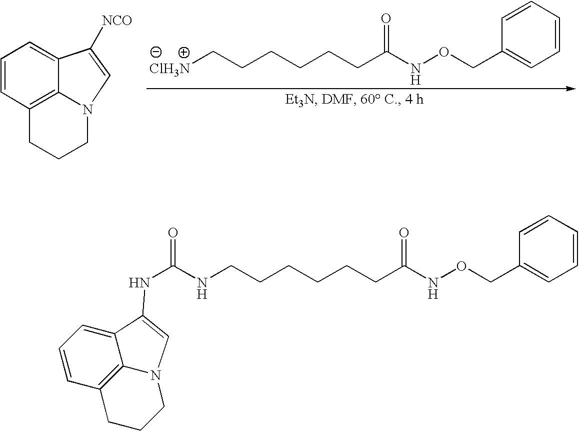

1-isocyanato-5,6-dihydro-4H-pyrrolo[3,2,1-ij]quinoline was reacted with 7-amino-N-(benzyloxy)heptanamide using the same procedure M. Yield=96%. LCMS: 449 [M+H].

### Example 8.3

### General Procedure N—Step 3

### Example 8.3.1

### Synthesis of 6-[(5,6-dihydro-4H-pyrrolo[3,2,1-ij]quinolin-1-ylcarbamoyl)amino]-N-hydroxyhexanamide

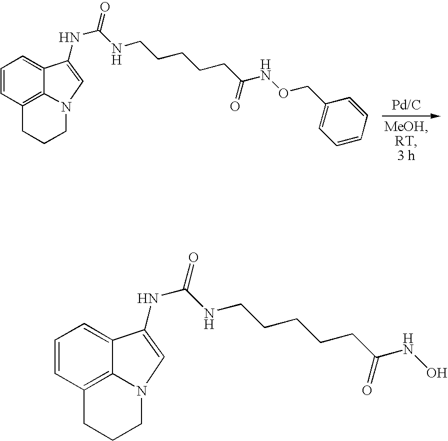

To a solution of the SM (0.20 g, 0.433 mmol) in methanol was added Pd/C (10%) (0.16 g). The reaction mixture was stirred at room temperature for 2-3 hours. Pd/C was filtered out and the solvent was removed under reduced pressure. The crude product was recrystalized using methanol and dichloromethane to afford the desired product as an off-white solid (0.047 mg, 30%). M.p.=161-163° C.; ¹H NMR (DMSO-d,₆) δ: 10.3(s, 1H), 8.66 (s, 1H), 7.93 (s, 1H), 7.82 (d, J=10.4 Hz, 1H), 7.75 (m, 1H), 7.01 (t, J=10.4 Hz, 1H), 6.89 (d, J=9.6 Hz, 1H), 4.18 (t, J=7.6 Hz, 2H), 3.22 (m, 2H), 2.92 (m, 2H), 2.12 (t, J=8 Hz, 2H), 1.95 (t, J=9.6 Hz, 2H), 1.52 (m, 2H), 1.28 (m, 2H); LCMS: 345 [M+H].

### Example 8.3.2

### Synthesis of 7-[(5,6-dihydro-4H-pyrrolo[3,2,1-ij]quinolin-1-ylcarbamoyl)amino]-N-hydroxyheptanamide

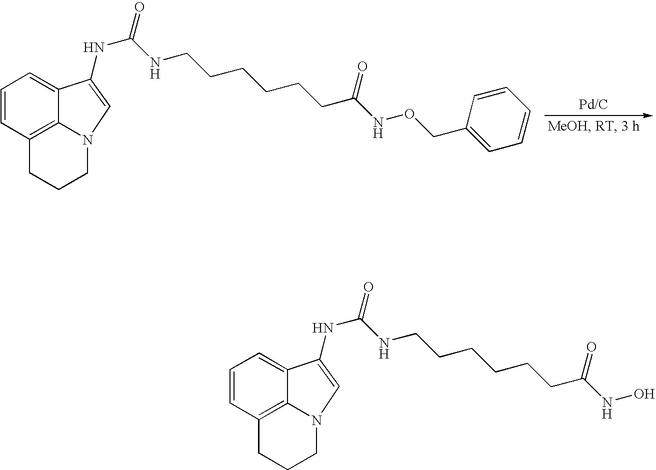

N-(benzyloxy)-7-[(5,6-dihydro-4H-pyrrolo[3,2,1-ij]quinolin-1-ylcarbamoyl)amino]heptanamide was subjected to hydrogenation conditions same as procedure N. Yield=75%. M.p.=185-187° C.; ¹H NMR (DMSO-d₆) δ: 10.3(s, 1H), 8.66 (s, 1H), 7.93 (s, 1H), 7.82 (d, J=10.4 Hz, 1H), 7.75 (m, 1H), 7.01 (t, J=10 Hz, 1H), 6.89 (d, J=9.6 Hz, 1H), 4.18 (t, J=8 Hz, 2H), 3.22 (m, 2H), 2.92 (t, J=8 Hz, 2H), 2.12 (m, 2H), 1.93 (t, J=10 Hz, 2H), 1.49 (m, 2H), 1.28 (m, 2H); LCMS: 359 [M+H].

### Example 9

### General Scheme 5

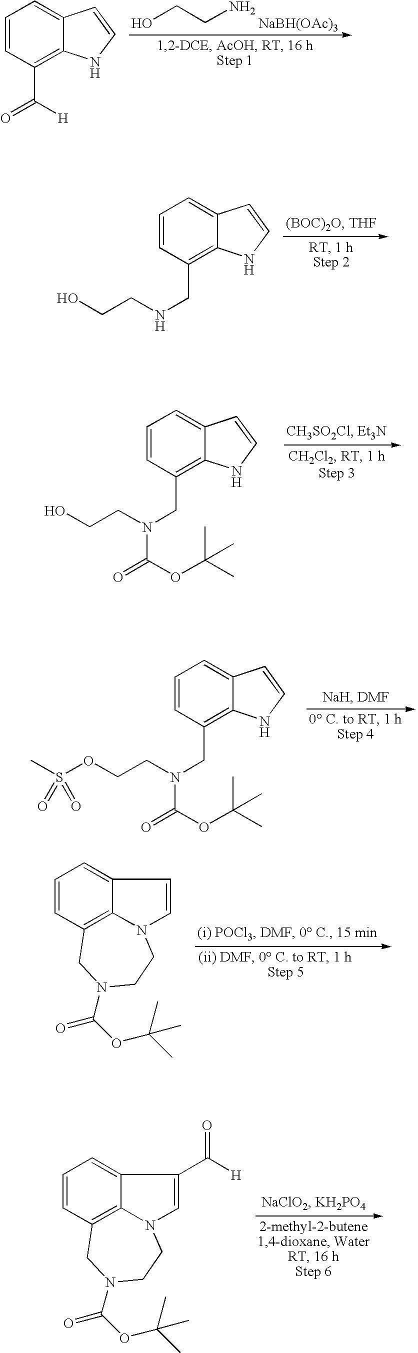

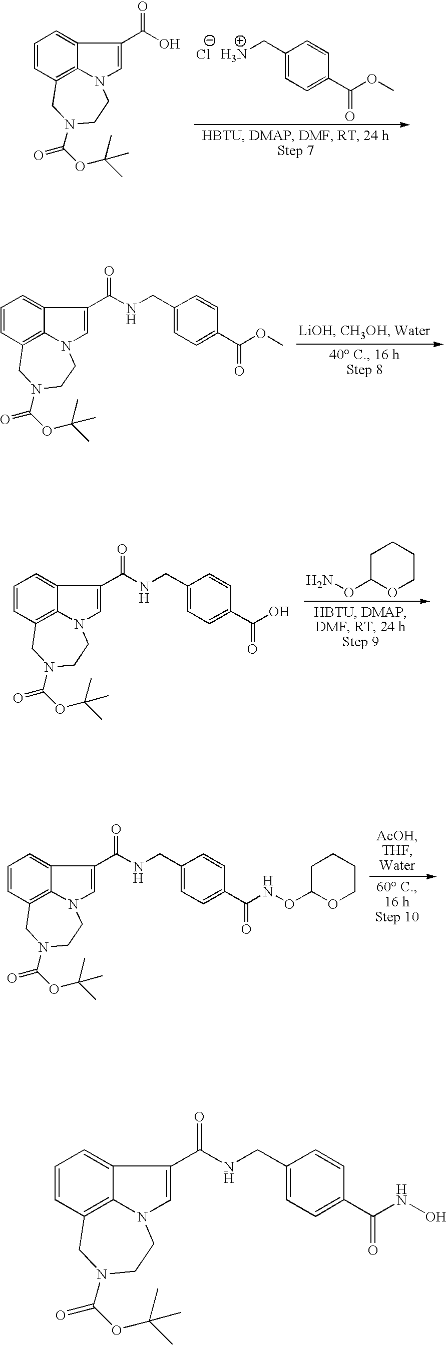

### Example 9.1

### Step 1

### Synthesis of 2-[(1H-indol-7-ylmethyl)amino]ethanol

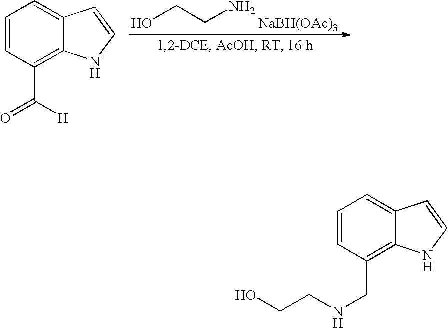

To a solution of 7-formyl indole (5.0 g, 34.5 mmol) in 1,2-dichloroethane (60 mL) was added aminoethanol (2.5 mL, 41.3 mmol) followed by glacial acetic acid (4.0 mL) and sodium triacetoxyborohydride (8.03 g, 37.9 mmol). The reaction was allowed to stir at room temperature for 16 hours. The reaction was quenched by addition of H₂O (10 mL) and 1.0N NaOH (10 mL). The organic layer was separated and the aqueous layer once again extracted with 1,2-dichloroethane (80 mL). The combined organic extracts was washed with saturated NaHCO₃ (2×60 mL), water (2×100 mL), dried over anhydrous sodium sulfate and evaporated to dryness. The desired crude product (4.65 g) was obtained as an oil and used in the next step without any further purification. LCMS=189 [M+H].

### Example 9.2

### Step 2

### Synthesis of tert-butyl(2-hydroxyethyl)(1H-indol-7-ylmethyl)carbamate

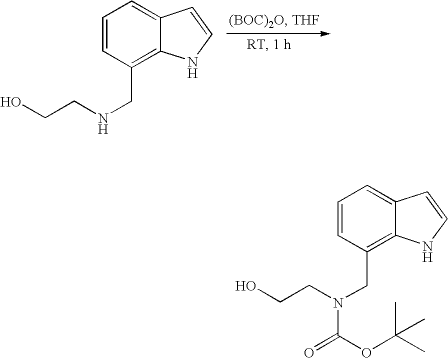

To a solution of the 2-[(1H-indol-7-ylmethyl)amino]ethanol (4.65 g, 23.9 mmol) in tetrahydrofuran (50 mL) was added BOC anhydride (5.73 g, 26.3 mmol). The reaction was allowed to stir at room temperature for 1 hour. The solvent was removed under reduced pressure and the residue dissolved in ethyl acetate (100 mL). The organic layer was washed with saturated sodium bicarbonate (100 mL), water (2×100 mL), dried with sodium sulfate and the solvent removed under reduced pressure. The crude product was purified by flash column chromatography (SiO₂, 20% EtOAc in hexanes to 40% EtOAc in hexanes) to afford 5.98 g pure final product as an oil. ¹H NMR (CDCl₃) 400 MHz δ 10.18 (br s, 1H), 7.64-7.60 (m, 1H), 7.2-7.25 (m, 1H), 7.08-7.0 (m, 2H), 4.71 (s, 2H), 3.73-3.65 (m, 2H), 3.38-3.30 (m, 2H), 1.50 (s, 9H); LCMS=291 [M+H].

### Example 9.3

### Step 3

### Synthesis of 2-[(tert-butoxycarbonyl)(1H-indol-7-ylmethyl)amino]ethyl methanesulfonate

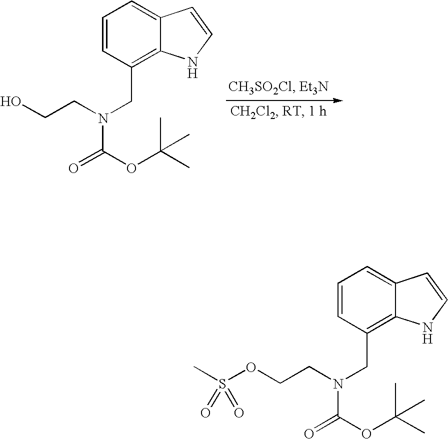

To a solution of the tert-butyl(2-hydroxyethyl)(1H-indol-7-ylmethyl)carbamate (6.71 g, 23.13 mmol) in dichloromethane (100 mL) was added triethylamine (3.89 mL, 27.77 mmol). The reaction mixture was cooled to 0° C. and the mesylchloride (2.0 mL, 25.45 mmol) added to the reaction mixture in a dropwise manner. The reaction was allowed to warmed to room temperature and allowed to stir for 2 hours. The reaction was quenched by adding water (50 mL) and 1.0 N NaOH (10 mL). The organic layer was separated and the aqueous layer extracted with dichloromethane (40 mL). The combined organic extract was washed with 1.0 N HCl (40 mL), water (70 mL), dried with sodium sulfate and the solvent removed under reduced pressure. The crude product (8.76 g) was isolated as an oil and used in the next reaction without any further purification. LCMS=370 [M+H].

### Example 9.4

### Step 4

### Synthesis of tert-butyl 3,4-dihydro[1,4]diazepino[6,7,1-hi]indole-2(1H)-carboxylate

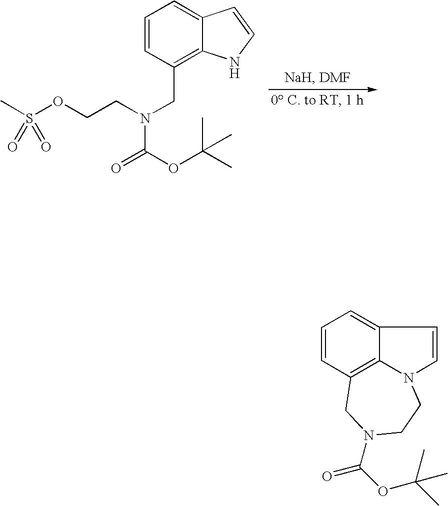

To a 0° C. cooled mixture of sodium hydride (60% dispersion in oil, 1.823 g, 47.6 mmol) in anhydrous DMF (60 mL) was a solution of 2-[(tent-butoxycarbonyl)(1H-indol-7-ylmethyl)amino]ethyl methanesulfonate (8.76 g, 23.8 mmol) in DMF (10 mL). The reaction mixture was warmed to room temperature and allowed to react for 1 hour. The reaction was quenched by adding water (200 mL). The aqueous layer was extracted with EtOAc (4×40 mL). The combined organic extract was washed with water (3×100 mL), dried with sodium sulfate and the solvent removed under reduced pressure. The crude product was purified by flash column chromatography (SiO₂, 100% dichloromethane) to afford 5.29 g (84% for two steps) of pure final product as an oil. ¹H NMR (CDCl₃) 400 MHz δ 7.58-7.5 (m, 1H), 7.1-6.92 (m, 3H), 6.53 (d, J=1.6 Hz, 1H), 4.89 (s, 1H), 4.81 (s, 1H), 4.3-4.21 (m, 2H), 4.0-3.9 (m, 2H), 1.5-1.4 (m, 9H); LCMS=273 [M+H].

### Example 9.5

### Step 5

### Synthesis of 7-Formyl-3,4-dihydro-1H-[1,4]-diazepino[6,7,1-hi]-indole-2-carboxylic acid tert-butyl ester

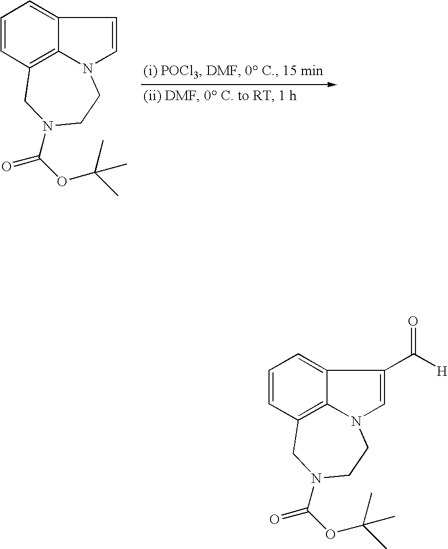

To a 0° C. cooled round bottom flask containing phosphoryl chloride (0.336 mL, 3.68 mmol) was added anhydrous DMF (1.0 mL, 18.4 mmol) in a dropwise manner The mixture was allowed to react at 0° C. for 15 minutes. To this solution was added a solution of tert-butyl 3,4-dihydro[1,4]diazepino[6,7,1-hi]indole-2(1H)-carboxylate (0.5 g, 1.84 mmol) in anhydrous DMF (5 mL). The reaction was allowed to warm to room temperature and stirred for 30 minutes. The reaction was quenched by adding saturated sodium bicarbonate (40 mL). The aqueous layer was extracted with EtOAc (3×30 mL). The combined organic extract was washed with water (3×40 mL), dried with sodium sulfate and the solvent removed under reduced pressure. The crude product (0.14 g) was isolated as an oil and used in the next reaction without any further purification. LCMS=302 [M+H].

### Example 9.6

### Step 6

### Synthesis of 2-(tert-butoxycarbonyl)-1,2,3,4-tetrahydro[1,4]diazepino[6,7,1-hi]indole-7-carboxylic acid

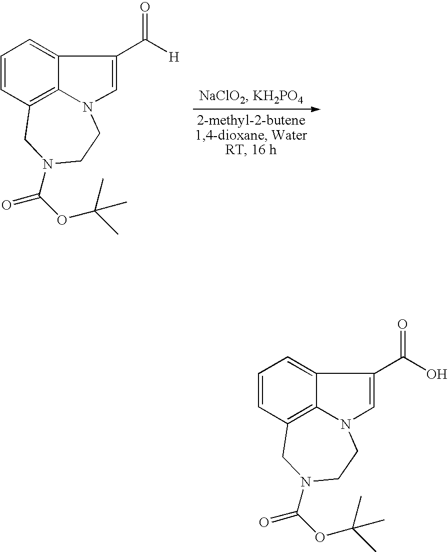

To a solution of the 7-Formyl-3,4-dihydro-1H-[1,4]-diazepino[6,7,1-hi]-indole-2-carboxylic acid tert-butyl ester (0.21 g, 0.70 mmol) in 1,4-dioxane (6.0 mL) was added a solution of sodium hypochlorite (1.899 g, 20.99 mmol), potassium dihydrogen phosphate (1.905 g, 13.99 mmol) in water (6.0 mL) followed by 2-methyl-2-butene (4.0 mL). The reaction mixture was stirred for 16 hours followed by addition of EtOAc (20 mL) and water (20 mL). The organic layer was separated and the aqueous layer extracted with EtOAc (20 mL). The combined organic extract was washed with water, dried with sodium sulfate and solvent removed under reduced pressure. The crude product was purified by flash column chromatography (SiO₂, 50% EtOAc in hexanes to 100% EtOAc) to afford 0.035 g of pure final product as a yellow powder. LCMS=318 [M+H].

### Example 9.7

### Step 7

### Synthesis of tent-butyl 7-{[4-(methoxycarbonyl)benzyl]carbamoyl}-3,4-dihydro[1,4]diazepino[6,7,1-hi]indole-2(1H)-carboxylate

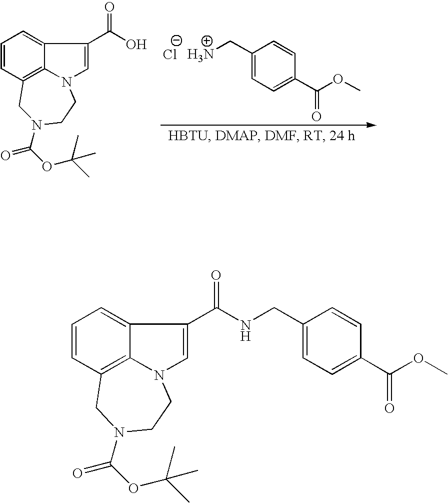

To a solution of the 2-(tert-butoxycarbonyl)-1,2,3,4-tetrahydro[1,4]diazepino[6,7,1-hi]indole-7-carboxylic acid (0.22 g. 6.98 mmol) in DMF (6.0 mL) was added HBTU (0.291 g, 7.68 mmol), and dimethylaminopyridine (0.128 g, 10.5 mmol) followed by methyl 4-aminomethylbenzoate hydrochloride (0.155 g, 7.68 mmol). The reaction was allowed to stir at room temperature for 24 hours. The reaction was quenched by adding water (50 mL). The aqueous layer was extracted with EtOAc (3×25 mL). The combined organic extract was washed with saturated sodium bicarbonate (2×50 mL), 1.0 N HCl (2×50 mL), water (2×50 mL), dried with sodium sulfate and the solvent removed under reduced pressure. The crude product was purified by flash column chromatography (SiO₂, 50% EtOAc in hexanes to 75% EtOAc in hexanes) to afford 0.18 g of pure final product as a yellow solid. ¹H NMR (CDCl₃) 400 MHz δ: 8.20 (d, J=4.3 Hz, 2H), 7.92-7.8 (m, 1H), 7.7-7.62 (m, 1H), 7.45 (d, J=4.3 Hz, 2H), 7.2-7.03 (m, 2H), 6.28-6.2 (m, 1H), 4.93 (s, 1H), 4.83 (s, 1H), 4.78-4.72 (m, 2H), 4.4-4.34 (m, 2H), 4.0-3.92 (m, 2H), 3.91 (s, 3H), 1.48-1.40 (m, 9H); LCMS=464 [M+H].

### Example 9.8

### Step 8

### Synthesis of 4-[({[2-(tert-butoxycarbonyl)-1,2,3,4-tetrahydro[1,4]diazepino[6,7,1-hi]indol-7-yl]carbonyl}amino)methyl]benzoic acid

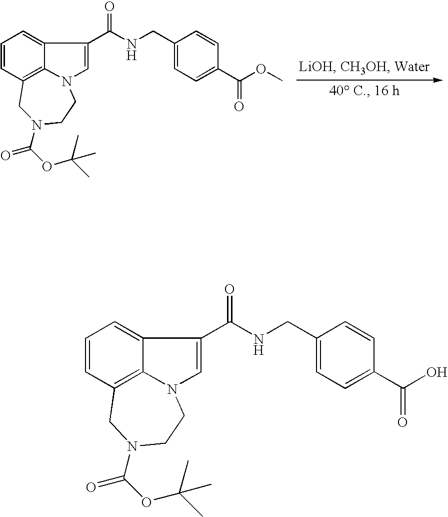

To a solution tert-butyl 7-{[4-(methoxycarbonyl)benzyl]carbamoyl}-3,4-dihydro[1,4]diazepino[6,7,1-hi]indole-2(1H)-carboxylate (0.18 g, 0.4 mmol) in methanol (4.0 mL) was added an solution of lithium hydroxide (0.015 g, 0.8 mmol) in water (4.0 mL). The reaction mixture was heated at 40° C. for 16 hours. The solvent was removed under reduced pressure and to the residue was added water (10 mL), dichloromethane (30 mL) and 1.0 N HCl (10 mL). The organic layer was separated and the aqueous layer was extracted with dichloromethane (20 mL). The combined organic extract was dried with sodium sulfate and the solvent removed under reduced pressure. The crude product (0.24 g) was isolated as a solid and used in the next reaction without any further purification. LCMS=450 [M+H].

### Example 9.9

### Step 9

### Synthesis of tent-butyl 7-({4-[(tetrahydro-2H-pyran-2-yloxy)carbamoyl]benzyl}carbamoyl)-3,4-dihydro[1,4]diazepino[6,7,1-hi]indole-2(1H)-carboxylate

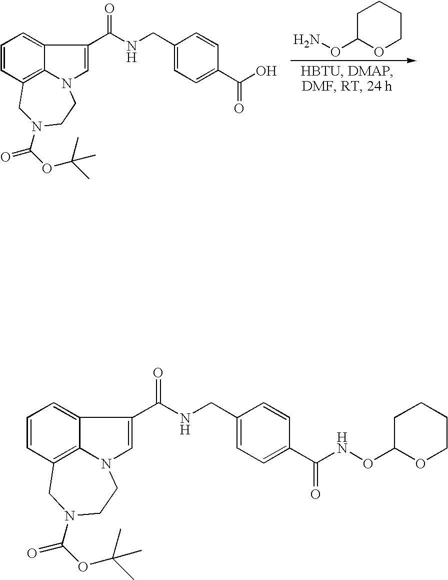

To a solution of the 4-[({[2-(tert-butoxycarbonyl)-1,2,3,4-tetrahydro[1,4]diazepino[6,7,1-hi]indol-7-yl]carbonyl}amino)methyl]benzoic acid (0.24 g, 0.53 mmol) in DMF (10 mL) was added HBTU (0.222 g, 0.58 mmol) and dimethlyaminopyridine (0.071 g, 0.58 mmol) followed by O-(tetrahydro-2H-pyran-2-yl)hydroxylamine 2-(aminooxy)tetrahydro-2H-pyran(0.094 g, 0.80 mmol). The reaction mixture was stirred for 18 hours. The reaction was quenched by adding water (50 mL). The aqueous layer was extracted with EtOAc (3×25 mL). The combined organic extract was washed with saturated sodium bicarbonate (2×20 mL), 1.0 N HCl (2×20 mL), water (2×50 mL), dried with sodium sulfate and the solvent removed under reduced pressure. The crude product was purified by flash column chromatography (SiO₂, 100% EtOAc) to afford 0.151 g of pure final product as a light yellow solid. ¹H NMR (CDCl₃) 400 MHz δ 9.16 (s, 1H), 8.0-7.8 (m, 1H), 7.7-7.62 (m, 3H), 7.4-7.33 (m, 2H), 7.2-7.0 (m, 2H), 6.6-6.43 (m, 1H), 5.08 (s, 1H), 4.91 (s, 1H), 4.82 (s, 1H), 4.68-4.61 (m, 2H), 4.38-4.23 (m, 2H), 4.07-4.0 (m, 1H), 3.97-3.91 (m, 2H), 3.68-3.6 (m, 1H), 1.97-1.8 (m, 3H), 1.75-1.56 (m, 3H), 1.5-1.38 (m, 9H); LCMS=465 [M+H].

### Example 9.10

### Step 10

### Synthesis of tert-butyl 7-{[4-(hydroxycarbamoyl)benzyl]carbamoyl}-3,4-dihydro[1,4]diazepino[6,7,1-hi]indole-2(1H)-carboxylate

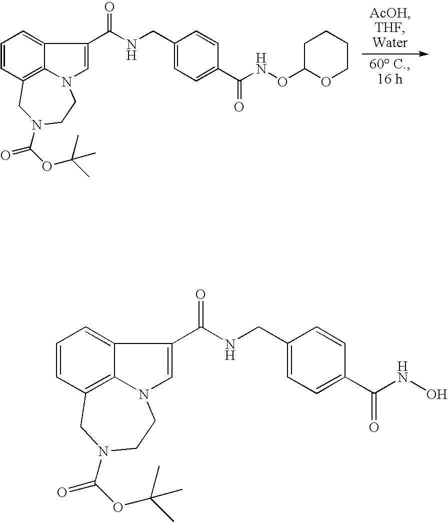

To a solution the tert-butyl 7-({4-[(tetrahydro-2H-pyran-2-yloxy)carbamoyl]benzyl}carbamoyl)-3,4-dihydro[1,4]diazepino[6,7,1-hi]indole-2(1H)-carboxylate (0.151 g, 0.28 mmol) in THF (1.5 mL) was added water (5.0 mL) and glacial acetic acid (2.0 mL). The reaction mixture was stirred at 60° C. for 16 hours. The solvent was then removed under reduced pressure. The crude product was purified by flash column chromatography (SiO₂, 5% methanol in dichloromethane to 10% methanol in dichloromethane) to afford 0.031 g of pure final product as a light yellow solid. ¹H NMR (Acetone-d₆) 400 MHz δ 10.76 (br s, 1H), 8.22-8.18 (m, 2H), 7.92 (s, 1H), 7.82-7.72 (m, 3H), 7.5-7.4 (m, 2H), 7.15-7.0 (m, 2H), 4.95-4.83 (m, 2H), 4.64 (s, 1H), 4.48 (s, 1H), 3.98 (s, 1H), 1.45-1.30 (m, 9H); LCMS=465 [M+H].

### Example 10

### HDAC Assay

A fluorescent biochemical assay has been developed to evaluate inhibitors of HDACs. The assay measures the ability of a small molecule to inhibit deacetylation of the substrate. Activator reagent recognizes the substrate only if the lysine has been deacetylated. Upon cleavage, the amino-coumarin is released, which can be detected fluorescently at 440-460 nm when excited at 350-380 nm (FIG. 1).

This homogeneous assay in performed in the same well without washing steps. The HDAC source, nuclear HeLa extract, is incubated with substrate in the presence of an inhibitor compound. At the end of the reaction time, activator solution, containing Trypsin and TSA, is added to stop the deacetylation reaction, and cleave the amino-coumarin from the deacetylated substrate. The plate is then read on either a Perkin Elmer Victor or Envision system using Umbilliferone filter set. Compounds preventing the HDAC from deactylating the peptide result in a lower fluorescent signal. The signal is directly proportional to the activity of HDACs and compound inhibition of HDAC is monitored by a decrease in signal.

### Procedure:

### 1. Reagents and Labware

96 well ½ area flat bottom white polystyrene plates were purchased from Corning (cat #3693). Trypsin was purchased from Sigma (cat #T-8802) and resuspended in 10 mg/mL in DPBS. Trichostatin A (TSA) was purchased from Upstate (cat #19-138) and resuspended in DMSO to stock concentration of 30 mM.

Substrate was synthesized in house. Stocks were prepared in DMSO (10 mM).

Assay buffer composition: 25 mM Tris (pH 8.0), 137 mM NaCl, 2.7 mM KCl, 1 mM MgCl₂.

### 2. Preparation of Working Solutions

HeLa extract working solution: 22.5 m/mL (1 μg/well)

Substrate working solution: 24.5μg/mL (100 μM in assay)

Compound working solution: Compounds dissolved in assay buffer at 4× Screening concentration. Activator solution: Trypsin (10 mg/mL) diluted 1:1600 in Assay Buffer containing 4 μM TSA.

### 3. Assay Conditions

Total reaction Volume: 40 μL

Reaction was allowed to run for 60 minutes at 37° C.

### 4. Assay Procedure

1. Thaw Assay Buffer, Substrate & TSA (all located in −20° C.) and keep on ice.

2. Combine in a white ½ area well plate: 10 μl of 4× compound (or buffer), 15 μl of substrate and 15 μl of HeLa extract. Mix well

3. Incubate at 37° C. for 60 min.

4. Prepare Activator solution. Dilute Trypsin 1:1600 in Assay Buffer+4μM TSA. 20 μl is needed per assay point.

5. Add 20 μl of prepared activator solution and mix thoroughly.

6. Read plate. Ex=350-380 nm, Em=440-460 nm.

The hydroxamic-acid based compounds of the present invention were tested in the biochemical assay for their pan-HDAC inhibitory activity. The results of the assay were shown in table?

### Example 11

### HDAC Isoform Assay

In addition to the pan-HDAC evaluations completed, several compounds of interest have been evaluated in parallel platform HDAC isoform assays. Assay procedures are identical to Pan HDAC; assay compositions are described as follows:

| | HDAC isoforms | | | | | | |
|---|---|---|---|---|---|---|---|
| Assay Condition | HDAC2 | HDAC3 | HDAC4 | HDAC5 | HDAC6 | HDAC8 | HDAC9 |
| ENZYME (final) | Commercial | Commercial | Commercial | Commercial | Commercial | Commercial | Commercial |
| | BioMol, | BioMol, | BioMol, | BioMol, | BioMol, | BioMol | BioMol, |
| | 0.02 ug/ul | 0.01 ug/ul | 0.03 ug/ul | 0.05 ug/ul | 0.03 ug/ul | | 0.05 ug/ul |
| SUBSTRATE (final) | KI-177, 5 uM | KI-178, 10 uM | KI-179, 10 uM | KI-177, | KI-177, 10.98 | KI-177 | KI-177, |
| | | | | 12.5 uM | uM | | 12.5 uM |

KI 177, KI-178 and KI179 were purchased form BPS biosciences.

The HDAC-1 assay represents a novel immunocapture assay procedure. A fluorescent biochemical assay has been developed to evaluate inhibitors of HDAC1. The assay measures the ability of a small molecule to inhibit deacetylation of the substrate by HDAC1 enzyme. The HDAC1 is captured on a protein A coated plate using HDAC1 specific antibody, and is then allowed to react with the substrate. Activator reagent recognizes the substrate only if the lysine has been deacetylated. Upon cleavage, the amino-coumarin is released, which can be detected fluorescently at 440-460 nm when excited at 350-380 nm.

This assay in performed in four general steps. In the first step (FIG. 1A), cell extract containing HDAC (HeLa in these experiments) is incubated with (cell signaling) rabbit polyclonal HDAC1 antibody (rabbit IgG antibody is used for background control) to allow binding of the enzyme to the antibody. In the second step, the complexed mixture is added to the protein A covered plate which captures the antibody. In the third step, after washing away unbound proteins, substrate and inhibitor are added and deacetylation is allowed to occur. At the end of the reaction time, activator solution, containing Trypsin and TSA, is added to stop the deacetylation reaction, and cleave the amino-coumarin from the deacetylated substrate. The plate is then read on either Victor or Perkin Elmer Envision system using Umbilliferone filter set. Compounds preventing the HDAC from deactylating the peptide result in a lower fluorescent signal. The signal is directly proportional to the activity of HDACs and compound inhibition of HDAC is monitored by a decrease in signal.

### 1. Reagents and Labware

96 well ½ area flat bottom white polystyrene plates were purchased from Corning (cat #3693).

96 well protein A coated plates were purchased from PIERCE (cat #15130).

Anti-HDAC1 antibody was purchased from Cell Signaling (cat #2062).

Trypsin was purchased from Sigma (cat #T-8802) and resuspended in 10 mg/mL in DPBS.

Trichostatin A (TSA) was purchased from Upstate (cat #19-138) and resuspended in DMSO to stock concentration of 30 mM.

Substrate was synthesized in house. Stocks were prepared in DMSO (10 mM).

Assay buffer composition: 25 mM Tris (pH 8.0), 137 mM NaCl, 2.7 mM KCl, 1 mM MgCl₂.

### 2. Preparation of Working Solutions

A. HeLa extract complexed with HDAC1 antibody working solution (in DPBS):

200 m/mL (10 μg/well) HeLa extract and 6.7μg/mL (0.335 m/well) Anti-HDAC1.
B. Substrate working solution (in HDAC buffer):

15 μg/mL (25 μM in assay; final concentration is 12.5 μM)
C. Compound working solution (in HDAC buffer):

Compounds diluted in assay buffer at 2× Screening concentration.
D. Activator solution:

Trypsin (10 mg/mL) diluted 1:1600 in assay buffer containing 4 μM TSA.

### 3. Assay Conditions

Complexing of extract with antibody was carried out at room temperature for 60 min with rocking. Binding of complexes onto protein A plate was carried out at room temperature for 60 min in 50 μL. Reaction was carried out in 50 IA, in the protein A plate for 60 minutes at 37° C. 40 μL of the reaction was moved into white ½ area plate for activation and reading.

### 4. Assay Procedure (for One Plate)

1. Set PIERCE Protein A plate to block with 150 μof 5% BSA in TB ST for 60 min at room temperature.
2. Combine per plate: 1000 μg of extract was incubated with 33.3 μg of antibody in 1500 μl final volume (in PBS) and incubate with gentle shaking (on a rocker) for 60 min at room temperature.
3. Dilute complexes further (2.3 fold) by addition of 3.5 mL of PBS (final total volume of 5 mL).
4. Wash blocked protein A plate prior to complex addition with TBST.
5. Add 50 μl of diluted complexes to Protein A plate and incubate for 60 min at room temperature.
6. Wash plate with TB ST.
7. Add 25 μl of diluted compounds (in HDAC buffer) to each well and incubate for 10 min at room temperature.
8. Add 25 μl of 25 μM substrate (final concentration is 12.5 μM) in HDAC buffer to the plate and incubate for 60 min at 37° C.
9. Move 40 μl of reaction to a white plate and add 20 μl of activator/developer solution, mix thoroughly.
10. Read plate. Ex=350-380 nm, Em=440-460 nm.

### Example 12

### MTS Assay

The MTS cell viability assay was used to determine the potency of proliferation inhibitor with. MTS measures mitochondrial dehydrogenase activity and serves as surrogate readout for the number of viable cells. The protocol described below is based upon the “CellTiter 96 Aqueous Non-Radioactive Cell Proliferation Assay” sold by Promega (Technical Bulletin No. 169).

### 1. Materials

| Material | Vendor |
|---|---|
| CellTiter 96 Aqueous MTS | Promega |
| Reagent Powder | |
| Phenazine methosulfate (PMS) | Fluka |
| 20% (w/v) SDS | Bio-Rad |
| DPBS (1X) | Invitrogen |
| DMSO | Sigma |

### 2. MTS Assays to Measure IC₅₀ for Cell Growth Inhibitors

For MTS assay, cells were plated in 96-well plates at 2000 cells per well and incubated in the presence of compounds for 72 hr. MTS was added to each well as instructed by manufacture (Promega) and plates were incubated for 4 h at 37° C. The absorbance of each well was measured at 490 nm using a microplate reader.

### Example 13

### p21 and Histone H4 Assays

HCT-116 cells were plated at approximately 60% confluency in 1 ml/media per well. Cells were treated with desired concentration of compound for 8 or 24 hours at 37 degrees C. in an incubator.

Lysates were generated by removing medial form cells and adding 150 μL 1× E-page Loading Buffer (Invitrogen) onto well. Wells were scraped into a microcentrifuge tube and sonicated 3× for 10-15 seconds. Samples were then heated to 70 degrees C. for 10 minutes and loaded onto Invitrogen E-page gels for separation and transfer to Nitrocellulose membrane. Western blotting was performed using anti-p21 or anti-acetylated histone H4 antibody, as well an anti-actin antibody for sample normalization. This was followed by detection with AlexaFluor 680 (Molecular Probes) or IRDYE800 (Rockland) secondary antibodies. Bolts were read on a LICOR Odyssey IR scanner.

Some of the data from experiments 11-13 are summarized in table 1.

Other embodiments are within the following claims. While several embodiments have been shown and described, various modifications may be made without departing from the spirit and scope of the present invention.

TABLE 1

| | | | HDAC2, | HDAC3, | HDAC4, | HDAC5, | HDAC6, |
|---|---|---|---|---|---|---|---|
| | | | IC50, | IC50, | IC50, | IC50, | IC50, |
| | | HDAC1 | ELISA | ELISA | ELISA | ELISA | ELISA |
| | | Lysate, | (KI- | (KI- | (KI- | (KI- | (KI- |
| cpd | | IC50, | 177) | 177) | 177) | 177) | 177) |
| # | Names | (uM) | (uM) | (uM) | (uM) | (uM) | (uM) |
| 1 | N-hydroxy-N′- | 0.092 | | 0.04 | | 0.544 | 0.262 |
| | phenyloctane | | | | | | |
| | diamide (SAHA) | | | | | | |
| 2 | N-[6- | 0.0132 | 1.94 | 0.0136 | | 0.0911 | 0.0672 |
| | (hydroxyamino)- | | | | | | |
| | 6-oxohexyl]-5,6- | | | | | | |
| | dihydro-4H- | | | | | | |
| | pyrrolo[3,2,1- | | | | | | |
| | ij]quinoline-1- | | | | | | |
| | carboxamide: | | | | | | |
| 3 | N-[7- | 0.041 | 2.46 | 0.0457 | 0.0405 | 0.095 | 0.158 |
| | (hydroxyamino)- | | | | | | |
| | 7-oxoheptyl]-5,6- | | | | | | |
| | dihydro-4H- | | | | | | |
| | pyrrolo[3,2,1- | | | | | | |
| | ij]quinoline-1- | | | | | | |
| | carboxamide | | | | | | |
| | N[8- | | | | | | |
| | (hydroxyamino)- | | | | | | |
| | 8-oxooctyl]-5,6- | | | | | | |
| | dihydro-4H- | | | | | | |
| | pyrrolo[3,2,1- | | | | | | |
| | ij]quinoline-1- | | | | | | |
| | carboxamide | | | | | | |
| 5 | N-[5- | 0.583 | | | | | |
| | (hydroxyamino)- | | | | | | |
| | 5-oxopentyl]-5,6- | | | | | | |
| | dihydro-4H- | | | | | | |
| | pyrrolo[3,2,1- | | | | | | |
| | ij]quinoline-1- | | | | | | |
| | carboxamide | | | | | | |
| 6 | N-{4- | 0.0259 | 3 | 0.0265 | | 0.0369 | 0.00782 |
| | [(hydroxyamino)- | | | | | | |
| | carbonyl]benzyl}- | | | | | | |
| | 5,6-dihydro-4H- | | | | | | |
| | pyrrolo[3,2,1- | | | | | | |
| | ij]quinoline-1- | | | | | | |
| | carboxamide: | | | | | | |
| 7 | 6-{[5,6-dihydro- | | | | | | No |
| | 4H-pyrrolo[3,2,1- | | | | | | data |
| | ij]quinolin-1- | | | | | | |
| | yl(oxo)acetyl]amino}- | | | | | | |
| | N- | | | | | | |
| | hydroxypropanamide | | | | | | |
| 8 | 6-{[5,6-dihydro- | 0.045 | 1.8 | 0.0204 | | 0.163 | 0.0776 |
| | 4H-pyrrolo[3,2,1- | | | | | | |
| | ij]quinolin-1- | | | | | | |
| | yl(oxo)acetyl]amino}- | | | | | | |
| | N- | | | | | | |
| | hydroxyhexanamide | | | | | | |
| 9 | 4-{[5,6-dihydro- | 0.583 | 3 | 0.63 | | | 0.692 |
| | 4H-pyrrolo[3,2,1- | | | | | | |
| | ij]quinolin-1- | | | | | | |
| | yl(oxo)acetyl]amino}- | | | | | | |
| | N- | | | | | | |
| | hydroxybutanamide | | | | | | |
| 10 | 4-{[5,6-dihydro- | | | | | | |
| | 4H-pyrrolo[3,2,1- | | | | | | |
| | ij]quinolin-1- | | | | | | |
| | yl(oxo)acetyl]amino}- | | | | | | |
| | N- | | | | | | |
| | hydroxypentanamide | | | | | | |
| 11 | 7-[(5,6-dihydro- | 0.022 | 1.34 | 0.023 | 0.0404 | 0.353 | 0.0664 |
| | 4H-pyrrolo[3,2,1- | | | | | | |
| | ij]quinolin-1- | | | | | | |
| | ylcarbamoyl)amino]- | | | | | | |
| | N- | | | | | | |
| | hydroxyheptanamide | | | | | | |
| 12 | 7-{[5,6-dihydro- | 0.0904 | 2.32 | 0.0876 | 0.298 | 0.26 | 0.134 |
| | 4H-pyrrolo[3,2,1- | | | | | | |
| | ij]quinolin-1- | | | | | | |
| | yl(oxo)acetyl]amino}- | | | | | | |
| | N- | | | | | | |
| | hydroxyheptanamide | | | | | | |
| 13 | 6-[(5,6-dihydro- | 0.0189 | 1.78 | 0.0205 | | 0.201 | 0.0632 |
| | 4H-pyrrolo[3,2,1- | | | | | | |
| | ij]quinolin-1- | | | | | | |
| | ylcarbamoyl)amino]- | | | | | | |
| | N- | | | | | | |
| | hydroxyhexanamide | | | | | | |
| 14 | N-benzyl-N-[7- | | | | | | |
| | (hydroxyamino)- | | | | | | |
| | 7-oxoheptyl]-5,6- | | | | | | |
| | dihydro-4H- | | | | | | |
| | pyrrolo[3,2,1- | | | | | | |
| | ij]quinoline-1- | | | | | | |
| | carboxamide | | | | | | |
| 15 | tert-butyl 7-{[4- | 0.492 | 10 | 0.518 | 0.791 | | 0.203 |
| | (hydroxycarbamoyl)benzyl]- | | | | | | |
| | carbamoyl}-3,4- | | | | | | |
| | dihydro[1,4]diazepino[6, | | | | | | |
| | 7,1- | | | | | | |
| | hi]indole-2(1H)- | | | | | | |
| | carboxylate | | | | | | |
| 16 | N-{4-[2- | | | | | | |
| | (hydroxyamino)- | | | | | | |
| | 2- | | | | | | |
| | oxoethyl]phenyl}- | | | | | | |
| | 5,6-dihydro-4H- | | | | | | |
| | pyrrolo[3,2,1- | | | | | | |
| | ij]quinoline-1- | | | | | | |
| | carboxamide | | | | | | |
| 17 | N-(6-oxoheptyl)- | | | | | | |
| | 5,6-dihydro-4H- | | | | | | |
| | pyrrolo[3,2,1- | | | | | | |
| | ij]quinoline-1- | | | | | | |
| | carboxamide | | | | | | |
| 18 | 3-[1-(5,6-dihydro- | 0.0357 | 10 | 0.0298 | 0.0691 | 0.0915 | 0.144 |
| | 4H-pyrrolo[3,2,1- | | | | | | |
| | ij]quinolin-1- | | | | | | |
| | ylcarbonyl)piperidin- | | | | | | |
| | 4-yl]-N- | | | | | | |
| | hydroxypropanamide | | | | | | |
| 19 | 4-[1-(5,6-dihydro- | | | | | | |
| | 4H-pyrrolo[3,2,1- | | | | | | |
| | ij]quinolin-1- | | | | | | |
| | ylcarbonyl)piperidin- | | | | | | |
| | 4-yl]butan-2- | | | | | | |
| | one | | | | | | |
| 20 | N-{7-[(2- | | | | | | |
| | aminophenyl)amino]- | | | | | | |
| | 7-oxoheptyl}- | | | | | | |
| | 5,6-dihydro-4H- | | | | | | |
| | pyrrolo[3,2,1- | | | | | | |
| | ij]quinoline-1- | | | | | | |
| | carboxamide | | | | | | |
| 21 | N-{7-[(2-amino- | | | | | | |
| | 4,5- | | | | | | |
| | dichlorophenyl)amino]- | | | | | | |
| | 7-oxoheptyl}- | | | | | | |
| | 5,6-dihydro-4H- | | | | | | |
| | pyrrolo[3,2,1- | | | | | | |
| | ij]quinoline-1- | | | | | | |
| | carboxamide | | | | | | |
| 22 | N-[7- | | | | | | |
| | (hydroxyamino)- | | | | | | |
| | 7-oxoheptyl]-6-(3- | | | | | | |
| | methoxyphenyl)imidazo[2, | | | | | | |
| | 1- | | | | | | |
| | b][1,3]thiazole-2- | | | | | | |
| | carboxamide | | | | | | |
| 23 | N-[7- | 0.017 | 3 | 0.037 | 0.028 | | 0.043 |
| | (hydroxyamino)- | | | | | | |
| | 7-oxoheptyl]-5,6- | | | | | | |
| | dihydro-4H- | | | | | | |
| | pyrrolo[3,2,1- | | | | | | |
| | ij]quinoline-2- | | | | | | |
| | carboxamide | | | | | | |

| | | | HeLa | | | | |
|---|---|---|---|---|---|---|---|
| | | | Extract, | HCT- | | | |
| | | | IC50, | 116, | HH, | Hut78, | MJ, |
| | HDAC8, | HDAC9, | (HDAC | IG50, | IG50, | IG50, | IG50, |
| cpd | IC50, | IC50, | Peptide) | MTS | MTS | MTS | MTS |
| # | (uM) | (uM) | (uM) | (uM) | (uM) | (uM) | (uM) |
| 1 | 1.65 | | 0.182 | 0.78 | | 1.02 | 1.65 |
| 2 | 1.09 | 0.106 | 0.0574 | 0.63 | 1.72 | 2.92 | 5 |
| 3 | 0.408 | 0.413 | 0.0698 | 0.215 | 0.111 | 0.24 | 0.12 |
| 5 | | | 0.809 | 5.81 | | | |
| 6 | 1.25 | | 0.0708 | 0.457 | 0.091 | | 0.58 |
| 7 | | | | | | | |
| 8 | 9.24 | | 0.115 | 0.737 | 2.62 | | 7.68 |
| 9 | 10 | | 0.602 | 5.02 | 0.365 | 0.903 | 4.11 |
| 10 | | | 11.3 | | | | |
| 11 | 0.492 | 0.0887 | 0.0279 | 0.492 | 0.094 | 0.181 | 0.47 |
| 12 | 6.42 | 1.06 | 0.126 | 0.701 | 0.045 | 0.174 | 0.13 |
| 13 | 0.918 | 0.0972 | 0.0322 | 0.636 | 1.02 | 1.27 | |
| 14 | | | 0.78 | 3.8 | | | |
| 15 | 3.45 | | 0.505 | | | | |
| 16 | | | 2 | | | | |
| 17 | | | 10 | | | | |
| 18 | 4.97 | 0.535 | | 1.14 | 0.186 | 0.987 | 0.39 |
| 19 | | | 20 | | | | |
| 20 | | | 4.39 | | | | |
| 21 | | | 15.5 | | | | |
| 22 | | | 0.487 | | | | |
| 23 | | | | | | | |

What is claimed is:

1. A compound of formula I 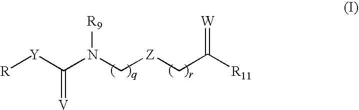
wherein
R is 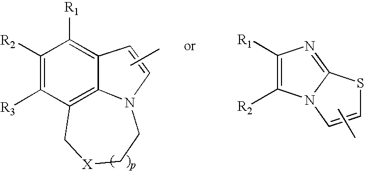
R₁, R₂, and R₃ are each independently selected from the group consisting of H, C₁-C₅ alkyl, C₁-C₅ substituted alkyl, aryl, halogen, —C(═O)NHR₄, and —C(═O)OR₄;
R₄ is H or C₁-C₅ alkyl, aryl, heteroaryl;
p and q are each independently selected from the group consisting of 0, 1, 2, and 3;
X is a bond, NR_{S}, or S or O;
R₅ is selected from the group consisting of H, alkyl, substituted alkyl, aryl, —CH₂-aryl, heteroaryl, —C(═O)R₆, —C(═O)OR₆, —C(═O)NR₆R₇, —S(═O)2R₆, —(CH₂)ₛOH, and —CH₂CHOHR₆;
R₆ is selected from the group consisting of alkyl, aryl, —CH₂-aryl, heteroaryl;
R₇ is H or C₁-C₅ alkyl; R₆ and R₇ can form a five to seven membered saturated ring;
s is selected from the group consisting of 0, 1, 2, 3, 4, and 5;
Y is a bond, C(═O), or NR₈;
R₈ is H or C₁-C₅ alkyl;
V and W are each independently O or S;
R₉ is selected from the group consisting of H, C₁-C₃ alkyl, aryl, and —CH₂-aryl; or R₉ can form a five or six membered saturated ring with R₁₀;
r is selected from the group consisting of 0, 1, 2, 3, 4, and 5;
Z is selected from the group consisting of a bond, —CHR₁₀, aryl, and alkylene;
R₁₀ is H or C₁-C₅ alkyl;
R₁₁ is —NR₁₂R₁₃, or C₁-C₄ alkyl; and
R₁₂ and R₁₃ are each independently selected from the group consisting of H, hydroxyl, substituted aryl, and heteroaryl.

2. The compound of claim 1 wherein R is

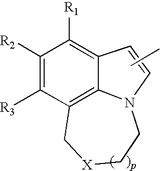

3. The compound of claim 2 wherein R₁, R₂, and R₃ are all H.

4. The compound of claim 2 wherein X is a bond and p is 1.

5. The compound of claim 2 wherein X is NR₂.

6. The compound of claim 1 wherein R is

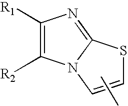

7. The compound of claim 6 wherein R₂ is H.

8. The compound of claim 1 wherein both V and W are O.

9. The compound of claim 1 wherein R₉ is H.

10. The compound of claim 1 wherein R₉ is —CH₂-aryl.

11. The compound of claim 1 wherein R₉ can form a six membered saturated ring with R₁₀.

12. The compound of claim 1 wherein Z is aryl.

13. The compound of claim 12 wherein Z is phenyl.

14. The compound of claim 1 wherein Z is a bond, q is 1 and r is 1, 2, 3, 4, or 5.

15. The compound of claim 1 wherein R₁₁ is —NR₁₂R₁₃.

16. The compound of claim 15 wherein R₁₂ is H.

17. The compound of claim 16 wherein R₁₃ is hydroxyl.

18. The compound of claim 16 wherein R₁₃ is substituted aryl.

19. The compound of claim 1 wherein R₁₁ is C₁-C₄ alkyl.

20. The compound of claim 1 wherein R₁₁ is methyl.

21. The compound of claim 1 wherein the compound is selected from the group consisting of N-[6-(hydroxyamino)-6-oxohexyl]-5,6-dihydro-4H-pyrrolo[3,2,1-ij]quinoline-1-carboxamide; N-[7-(hydroxyamino)-7-oxoheptyl]-5,6-dihydro-4H-pyrrolo[3,2,1-ij]quinoline-1-carboxamide; N-[8-(hydroxyamino)-8-oxooctyl]-5,6-dihydro-4H-pyrrolo[3,2,1-ij]quinoline-1-carboxamide; N-[5-(hydroxyamino)-5-oxopentyl]-5,6-dihydro-4H-pyrrolo[3,2,1-ij]quinoline-1-carboxamide; N-{4-[(hydroxyamino)carbonyl]benzyl}-5,6-dihydro-4H-pyrrolo[3,2,1-ij]quinoline-1-carboxamide; 6-{[5,6-dihydro-4H-pyrrolo[3,2,1-ij]quinolin-1-yl(oxo)acetyl]amino}-N-hydroxypropanamide, 6-{[5,6-dihydro-4H-pyrrolo[3,2,1-ij]quinolin-1-yl(oxo)acetyl]amino}-N-hydroxyhexanamide; 4-{[5,6-dihydro-4H-pyrrolo[3,2,1-ij]quinolin-1-yl(oxo)acetyl]amino }-N-hydroxybutanamide; 4-{[5,6-dihydro-4H-pyrrolo[3,2,1-ij]quinolin-1-yl(oxo)acetyl]amino}-N-hydroxypentanamide; 7-[(5,6-dihydro-4H-pyrrolo[3,2,1-ij]quinolin-1-ylcarbamoyl)amino]-N-hydroxyheptanamide; 7-{[5,6-dihydro-4H-pyrrolo[3,2,1-ij]quinolin-1-yl(oxo)acetyl]amino}-N-hydroxyheptanamide; 6-[(5,6-dihydro-4H-pyrrolo[3,2,1-ij]quinolin-1-ylcarbamoyl)amino]-N-hydroxyhexanamide; N-benzyl-N-[7-(hydroxyamino)-7-oxoheptyl]-5,6-dihydro-4H-pyrrolo[3,2,1-ij]quinoline-1-carboxamide; tert-butyl 7-{[4-(hydroxycarbamoyl)benzyl]carbamoyl}-3,4-dihydro[1,4]diazepino[6,7,1-hi]indole-2(1H)-carboxylate; N-{4-[2-(hydroxyamino)-2-oxoethyl]phenyl}-5,6-dihydro-4H-pyrrolo[3,2,1-ij]quinoline-1-carboxamide; N-(6-oxoheptyl)-5,6-dihydro-4H-pyrrolo[3,2,1-ij]quinoline-1-carboxamide; 3-[1-(5,6-dihydro-4H-pyrrolo[3,2,1-ij]quinolin-1-ylcarbonyl)piperidin-4-yl]-N-hydroxypropanamide; 4-[1-(5,6-dihydro-4H-pyrrolo[3,2,1-ij]quinolin-1-ylcarbonyl)piperidin-4-yl]butan-2-one; N-{7-[(2-aminophenyl)amino]-7-oxoheptyl}-5,6-dihydro-4H-pyrrolo[3,2,1-ij]quinoline-1-carboxamide; N-{7-[(2-amino-4,5-dichlorophenyl)amino]-7-oxoheptyl}-5,6-dihydro-4H-pyrrolo[3,2,1-ij]quinoline-1-carboxamide; N-[7-(hydroxyamino)-7-oxo heptyl]-6-(3-methoxyphenyl)imidazo[2,1-b][1,3]thiazole-2-carboxamide; and N-[7-(hydroxyamino)-7-oxoheptyl]-5,6-dihydro-4H-pyrrolo[3,2,1-ij]quinoline-2-carboxamide.

22. A pharmaceutical composition comprising a compound of claim 1 in combination with a pharmaceutically acceptable carrier or excipient.

23. The pharmaceutical composition of claim 22 further comprising a second chemotherapeutic agent.

24. The pharmaceutical composition of 23, wherein said second chemotherapeutic agent is selected from the group consisting of tamoxifen, raloxifene, anastrozole, exemestane, letrozole, cisplatin, carboplatin, paclitaxel, cyclophosphamide, lovastatin, minosine, gemcitabine, araC, 5-fluorouracil, methotrexate, docetaxel, goserelin, vincristin, vinblastin, nocodazole, teniposide, etoposide, epothilone, navelbine, camptothecin, daunonibicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab.

25. A method of treating a cell proliferative disorder, said method comprising administering to a subject in need thereof a therapeutically effective amount of a compound of formula I as defined in claim 1, or a pharmaceutically acceptable salt thereof, or a prodrug or metabolite thereof, in combination with a pharmaceutically acceptable carrier, wherein said cell proliferative disorder is treated.

26. The method of claim 25, wherein said cell proliferative disorder is a precancerous condition.

27. The method of claim 25, wherein said cell proliferative disorder is a cancer.

28. The method of claim 25, wherein said cancer is adenocarcinoma, squamous carcinoma, sarcoma, lymphoma, multiple myeloma, or leukemia.

29. The method of claim 25, wherein said cancer is lung cancer, colon cancer, breast cancer, pancreatic cancer, prostate cancer, acute leukemia, chronic leukemia, multiple melanoma, ovarian cancer, malignant glioma, leiomyosarcoma, hepatoma, or head and neck cancer.

30. The method of claim 25, wherein said compound of formula I, or a pharmaceutically acceptable salt thereof, or a prodrug or metabolite thereof, is administered in combination with a second chemotherapeutic agent.

31. The method of claim 30, wherein said second chemotherapeutic agent is selected from the group consisting of tamoxifen, raloxifene, anastrozole, exemestane, letrozole, cisplatin, carboplatin, paclitaxel, cyclophosphamide, lovastatin, minosine, gemcitabine, araC, 5-fluorouracil, methotrexate, docetaxel, goserelin, vincristin, vinblastin, nocodazole, teniposide, etoposide, epothilone, navelbine, camptothecin, daunonibicin, dactinomycin, mitoxantrone, amsacrine, doxorubicin, epirubicin, idarubicin imatanib, gefitinib, erlotinib, sorafenib, sunitinib malate, trastuzumab, rituximab, cetuximab, and bevacizumab.

32. The method of claim 25, wherein said treating cancer comprises a reduction in tumor size, a delay of tumor growth, an improvement in the survival of patients, or an improvement in the quality of patient life.

33. The method of claim 25, wherein the cancer is primary cancer or metastatic cancer.

34. A method of treating central nervous system (CNS) disorder, said method comprising administering to a subject in need thereof a therapeutically effective amount of a compound of formula I as defined in claim 1, or a pharmaceutically acceptable salt thereof, or a prodrug or metabolite thereof, in combination with a pharmaceutically acceptable carrier, wherein said central nervous system disorder is treated.

35. The method of claim 34 wherein the central nervous system disorder is selected from the group consisting of Rat's syndrome, the mental retardation-associated Rubinstein-Taybi syndrome, spinal muscular atrophy (SMA), motor neuron disease, Huntington's disease, Parkinson's disease (PD), and Alzhimer's disease.
